(19) United States
(12) Patent Application Publication
(10) Pub. No.: US 2026/0367100 A1
(43) Pub. Date: Nov. 19, 2026

(54) METHODS FOR DETERMINING THE HEALTH STATUS OF FISH

(71) Applicant: WELLFISH TECH LIMITED, Paisley (GB)

(72) Inventors: Josip BARISIC, Bridge of Weir (GB); Kevin ABOUA, Bridge of Weir (GB); Brian QUINN, Bridge of Weir (GB)

(21) Appl. No.: 19/131,826

(22) PCT Filed: Nov. 21, 2023
(86) PCT No.: PCT/GB2023/053044

§ 371 Date: May 21, 2025

(30) Foreign Application Priority Data
Nov. 22, 2022 (GB) 2217472.6

(51) Int. Cl. G01N 33/84; A01K 61/95; C12Q 1/32; C12Q 1/34; C12Q 1/48; C12Q 1/52; G01N 33/92

(52) U.S. Cl. CPC G01N 33/84; A01K 61/95; C12Q 1/32; C12Q 1/34; C12Q 1/485; C12Q 1/52; G01N 33/92; G01N 2333/4603

(57) ABSTRACT

The present invention relates to methods for determining the health status of populations of fish and diagnosing conditions or diseases in unhealthy fish. In particular, the present invention relates to blood biomarkers for assessing the health status and diagnosing conditions and diseases in populations of fish. The present invention also provides a method of determining a welfare score for a population of fish.

### FIELD OF THE INVENTION

The present invention relates to biomarkers and particular methods for identifying the health status of fish populations, preferably but not limited to, farmed fish. The present invention further relates to diagnosing a condition or disease and/or providing a welfare score for a population of fish. Some methods of the present invention relate to computer implemented methods.

### BACKGROUND OF THE INVENTION

Aquaculture is the fastest growing food supply sector in the world. The farming of aquatic animals grew on average 5.3% per year between 2001 and 2018. In 2018, the world's total aquaculture production reached an all-time high of 114.5 million tonnes in live weight with a total estimated first-sale value of US dollars (US$) 263 billion. Finfish accounted for 54 million tonnes with a value of $139.7 billion (FAO, 2020).

The international Salmonid industry is worth an estimated $15.4 billion (2017) with the main production sites in Norway, Chile, Scotland and Canada (FAO, 2020). The total supply of all farmed salmonids exceeded 2.2 million tonnes (gross weight) in 2018, more than double the wild catch and increased with a 7% compound annual growth rate in 2019 to just over 2.6 million tonnes (FAO, 2020).

Good fish health is of obvious importance to the salmon aquaculture industry, helping to reduce expenditure and increase productivity and ultimately profitability. Globally, mortality in salmon farming is estimated to be around 20%, greatly impacting on profitability and creating a negative public perception of the industry. Of the 48 million smolts put to sea in Scotland in 2014, 9 million (26.7%) died during the two-year production cycle, with mortality numbers reaching 10 and 11 million for 2016 and 2017 respectively (Munro L. A. & Wallace, I. S., 2017). In Norway a mortality rate of 19% (53 million salmon) in 2016 cost the industry NOK10 billion (~£1 billion).

Currently fish health managers have no method to rapidly assess fish health and are reliant on slow, lethal and manual techniques that can take up to 10 days to provide results. By this time the problem may have spread throughout the site, making it more difficult to treat, and potentially contributing to high mortality rates.

Clinical blood biochemistry has been routinely used in human and veterinary medicine to determine the health status of humans and animals. However, various issues, including a combination of a lack of established normal reference levels and a lack of clinically significant data has meant the use of clinical biochemistry has not yet been widely utilised within the aquaculture industry. The inventors of the present invention have generated significant healthy and diseased datasets to overcome both of these issues.

A recent study has produced a profile of blood biomarkers in intensively farmed Salmonid species (spp.) at different stages of the Salmonid life cycle, namely pre-smolt, smolt, post-smolt and adults. The aim of this study was to provide reference intervals for blood biomarkers during the freshwater (pre-smolt and smolt) and saltwater (post-smolt and adult) stages of development (Marco Rozas-Serri, 2022). This study provides an indication of blood biomarker reference intervals in health at different stages of development in health but provides no indication of how blood biomarkers change in disease or how different diseases can be diagnosed from blood biomarkers alone.

Therefore, there remains a need in the aquaculture industry for the rapid assessment of fish health without the need for lethal or slow techniques. The generation of both healthy and diseased datasets has allowed the present inventors to devise a comprehensive framework through both clinical analysis and artificial intelligence modelling to not only identify the health status of a fish but also diagnose a number of health challenges based on biomarker expression and provide population wide predictions on fish welfare.

Accordingly, in at least some aspects the present disclosure describes a method for determining the health status of a population of fish using a rapid blood test. This disruptive technology has been developed to augment, and may ultimately replace, reliance on lethal histological methods and enable predictive fish health forecasting using a novel data informed, pro-active healthcare model that results in increased productivity and reduced mortality in aquaculture.

### BRIEF SUMMARY OF THE INVENTION

The present invention relates to providing methods for determining the health status and/or diagnosing a condition or disease in a population of fish based on blood biomarker (analyte) amounts (e.g. expression levels). Computer implemented methods for identifying and predicting the health status, disease diagnosis and welfare of populations of fish based on models trained on blood biomarker amounts are also provided herein. Methods of the invention may also include treating fish to improve their health or taking other appropriate action in view of an indicated health status or condition or disease. The invention also relates, in part, to various combinations biomarkers and their use in methods to determine fish health status or to diagnose various conditions or diseases.

Accordingly, in a first aspect of the invention, there is provided a method for determining the health status of at least one fish in a population of fish, comprising the steps of: a) analysing one or more first samples collected from at least one fish of the population of fish to determine the amount of at least one analyte selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium present in the sample to determine a test profile; and
b) comparing the amount of the at least one analyte present in the test profile with a reference profile;
wherein a difference in the amount of the at least one analyte in the test profile as compared to the reference profile indicates the health status of the at least one fish in a population of fish.

Suitably, in some embodiments the health status of the fish is healthy or unhealthy.

In some embodiments the method is for determining the health status of a population of fish by determining the health status at least one fish within the population of fish, e.g., one or more fish that are representative of the population or from which the health status of the population can be inferred. Thus, in some embodiments, samples from at least one fish in a population (typically a plurality of fish in a population) can be analysed to determine the overall health status of a population of fish. In some embodiments the method can be used to determine the health status of one or more individual fish within a population.

A population of fish may comprise at least one, at least two, at least five, at least ten, at least one hundred, at least a thousand fish, at least five thousand, at least ten thousand fish, at least twenty thousand fish, at least fifty thousand fish, at least one hundred thousand fish or at least five hundred thousand fish. In some embodiments, a population of fish may refer to a plurality of fish. The population of fish may include fish in one or more enclosures (e.g., pens, cages or tanks). In some embodiments, the population of fish may be wild, captive or farmed fish. In some embodiments, the population of fish are salmonid, sea bass, sea bream, sturgeon, tilapia and/or carp. Preferably, the population of fish is a population of salmon or trout. In some embodiments, the population of fish are shellfish, for example, shrimp, oysters, lobsters, crabs, mussels and clams.

The method suitably comprises analysing samples obtained from a least 1, 2, 3, 4, 5, 6, 7, 8, 9, 10, 20, 30, 40, 50, 60, 70, 80, 90, 100, 200, 500, or 1000 fish from the population of fish. In some embodiments samples from a plurality of fish are analysed to provide an indication of the health status the population of fish. In some embodiments samples from at least 0.001%, 0.005%, 0.01%, 0.05%, or 0.1% of the total number of fish in the population are analysed.

Thus, the method of the invention can be used to determine the overall health status of a population of fish by sampling a sub-population of the total population of fish.

The term ‘analyte’ or ‘biomarker’ as used interchangeably herein means any entity, particularly a chemical, biochemical or biological entity to be assessed, e.g., whose amount (e.g., concentration or mass), activity, composition, or other properties are to be detected, measured, quantified, evaluated, analysed, etc. An ‘analyte’ or a ‘biomarker’, generally refers to a qualitative and/or quantitative measurable indicator of some biological state or condition.

Biomarkers are typically molecules, biological species or biological events that can be used for the detection, diagnosis, prognosis and prediction of therapeutic response of diseases.

Various preferred combinations of biomarkers for use in the present invention are discussed herein.

The test profile may refer to the amount (e.g., concentration or mass) of any one or more analytes as determined in a sample. In some preferred embodiments the amount is the concentration of any one or more analytes in a sample. Suitably, in some embodiments the test profile refers to the amount of any one or more analytes in the first sample, second sample, third sample or any later sample obtained from the population of fish.

Reference profile as used herein suitably refers to a predetermined profile of one or more analytes which is indicative of a healthy or unhealthy status. Thus, comparison of a test profile to the reference profile allows the identification of one or more analytes that differs from the reference profile. The reference profile may refer to the amount of any analyte in a fish or a population of fish or a sample collected from said fish or population of fish. Suitably, in some embodiments the reference profile refers to the amount of any one or more analytes in a healthy or unhealthy fish or population of fish. In some embodiments, the reference profile may refer to the amounts of a plurality of analytes in a healthy or unhealthy fish or population of fish. For example, the reference profile may comprise the amounts of a plurality of analytes representative of a healthy fish or population of fish or a plurality of analytes representative of an unhealthy fish or population of fish. Where a test profile differs from a reference profile indicative of a healthy fish, this is indicative of an unhealthy status. Where a test profile differs from a reference profile indicative of an unhealthy fish, this can be indicative of a healthy status. In some preferred embodiments the test profile is compared to a reference profile indicative of a healthy status.

In some embodiments the plurality of analytes in the reference and/or test profile incudes at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least forty, at least fifty, at least sixty, at least seventy, at least eighty, at least ninety, at least one hundred analytes representative of a healthy or unhealthy population of fish.

In some embodiments the reference and/or test profile comprises amounts of 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer or 2 or fewer analytes. In some embodiments the reference and/or test profile comprises the amount of 1 analyte. Suitably these analytes are selected from the group consisting of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium.

In some preferred embodiments the reference and/or profile and test profile comprise amounts for 18 analytes, e.g. chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. This set of 18 analytes has been determined to perform particularly well in various methods discussed herein. However, the invention is not intended to be limited to this set of analytes, and various subsets of analytes have been determined to be eminently suitable to determine health status and other conditions or diseases discussed herein.

Typically a test profile is compared to a corresponding reference profile, i.e., a reference profile relating to the same analytes analysed in the test sample. Suitably the corresponding reference profile relates to the same species of fish as the test profile. Suitably the corresponding reference profile relates to the same species of fish as the test profile, and which are exposed to identical or similar environmental conditions. The corresponding reference profile can correlate to a healthy or an unhealthy status.

In some embodiments, the reference profile may be established by obtaining more than one sample from a fish or from a population of fish over a time course. Suitably, in some embodiments, the reference profile is established by sampling a fish or a population of fish weekly, bi-weekly, monthly, quarterly or annually to determine a representative amount of the at least one analyte in a fish, a population of healthy fish and/or a population of unhealthy fish. In some embodiments, the one or more samples are obtained from fish from multiple sites. Multiple sites as referred to herein, may refer to fish or a population of fish housed in different enclosures on the same sampling site (i.e., on the same fish farm or the same body of water). Alternatively, multiple sites may refer to fish or a population of fish housed in independent sampling sites (i.e., on different fish farms or in different bodies of water).

Suitably, the representative amount may be referred to as the background level of the fish or the population of fish. Suitably, background levels as used herein may refer to the amount of each analyte in a representative healthy fish or the average amount of each analyte determined from a population of healthy fish. Alternatively, background levels as used herein may refer to the amount of each analyte in a representative unhealthy fish or the average amount of each analyte determined from a population of unhealthy fish.

In some embodiments, the reference profile may be determined from a fish or a population of fish, wherein the fish or the population of fish are members of the salmonid, cichlidae, carp or acipenseridae families. In some embodiments the fish or population of fish are shellfish. Suitable shellfish may include shrimp, oysters, lobsters, crabs, mussels and clams.

Suitably, in some embodiments, the reference profile may be the test profile obtained for a comparative population of fish. A comparative population of fish may include a population of fish of the same or different species, a population of fish housed under similar animal husbandry conditions (e.g., parasite treatment regimens), similar population numbers per enclosure, similar developmental stage of the fish (e.g., pre-smolt, smolt, post-smolt or adult) or similar environmental conditions (e.g., water temperature and time of year sample collected). Suitably, for example the reference profile may be the test profile obtained from a first sample collected from a population of fish, which acts as a baseline for comparison with subsequent samples. In some other embodiments, the reference profile may be the test profile obtained for the same or a different population of fish, e.g., at an earlier time point. This can suitably provide a reference profile closely corresponding to a test profile.

In any aspect of the invention described herein, two or more, three or more, four or more, five or more, six or more, seven or more, eight or more, nine or more, ten or more, eleven or more, twelve or more, thirteen or more, fourteen or more, fifteen or more, sixteen or more, seventeen or more or eighteen analytes may be analysed to determine the test and/or reference profile. In some embodiments, the test and/or reference profile comprises analysing a plurality of analytes. In some embodiments, the analytes may be selected from the list comprising: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. In some embodiments all of the recited analytes may be analysed to determine the test and/or reference profile.

Suitably, in some embodiments of any aspect of the present invention, the reference profile is used to determine one or more analyte reference ranges. Suitably, such ranges can be conceptualised or represented as unhealthy, abnormal and healthy ranges for a given analyte. Unhealthy may be further divided into low unhealthy which represents an analyte amount that is lower than the healthy analyte reference range and high unhealthy which represents an analyte amount that is higher than the healthy analyte reference range. Abnormal may be further divided into low abnormal which represents an analyte amount that is lower than the healthy analyte reference range but an analyte amount that is higher than the low unhealthy analyte reference range and high abnormal which represents an analyte amount that is higher than the healthy analyte reference range but an analyte amount that is lower than the high unhealthy analyte reference range. Such analyte reference ranges can be conceptualised or represented as a “traffic light” system, with healthy range indicated by green, abnormal (high and low) range indicated by amber and unhealthy (high and low) range indicated by red. In some embodiments, where more than one analyte is analysed in the test profile, a fish is classified as unhealthy when at least one of the analytes falls in the unhealthy range. In some embodiments, where more than one analyte is analysed in the test profile, a fish is classified as healthy if all analytes are in the healthy range. In some embodiments when one or more analytes are in the abnormal range a fish may be classified as having a health status that is healthy or unhealthy, depending on the number of analytes in the abnormal range.

For example, in some embodiments a representative healthy reference range of albumin in a blood sample from Salmonids is any amount of albumin between 15 and 21.2 g/L (green), the high abnormal range is any amount of albumin between 21.2 and 24.3 g/L (amber), the low abnormal range is any amount of albumin between 11.9 and 15 g/L, the low unhealthy range is any amount of albumin between 8.8 and 11.9 g/L and the high unhealthy range is any amount of albumin between 24.3 and 27.4 g/L (red).

In some embodiments a representative healthy reference range of alanine aminotransferase in a blood sample from Salmonids is any amount of alanine aminotransferase between 7.9 and 18.3 U/L (green), the high abnormal range is any amount of alanine aminotransferase between 18.3 and 23.5 U/L (amber), the low abnormal range is any amount of alanine aminotransferase between 2.7 and 7.9 U/L, the low unhealthy range is any amount of alanine aminotransferase below 2.7 U/L and the high unhealthy range is any amount of alanine aminotransferase between 23.5 and 28.7 U/L (red).

In some embodiments a representative healthy reference range of aspartate aminotransferase in a blood sample from Salmonids is any amount of aspartate aminotransferase between 368.3 and 664.9 U/L (green), the high abnormal range is any amount of aspartate aminotransferase between 664.9 and 813.2 U/L (amber), the low abnormal range is any amount of aspartate aminotransferase between 220 and 368.3 U/L, the low unhealthy range is any amount of aspartate aminotransferase below 220 U/L (red) and the high unhealthy range is any amount of aspartate aminotransferase between 813.2 and 961.5 U/L (red).

In some embodiments a representative healthy reference range of calcium in a blood sample from Salmonids is any amount of calcium between 2.8 and 3.4 mmol/L (green), the high abnormal range is any amount of calcium between 3.4 and 3.7 mmol/L (amber), the low abnormal range is any amount of calcium between 2.5 and 2.8 mmol/L (amber), the low unhealthy range is any amount of calcium below 2.5 mmol/L (red) and the high unhealthy range is any amount of calcium between 3.7 and 4 mmol/L (red).

In some embodiments, a representative healthy reference range of total cholesterol in a blood sample from Salmonids is any amount of total cholesterol between 6.5 and 12.5 mmol/L (green), the high abnormal range is any amount of total cholesterol between 12.5 and 15.5 mmol/L (amber), the low abnormal range is any amount of total cholesterol between 3.5 and 6.5 mmol/L (amber), the low unhealthy range is any amount of total cholesterol below 3.5 mmol/L (red) and the high unhealthy range is any amount of total cholesterol between 15.5 and 18.5 mmol/L (red).

In some embodiments a representative healthy reference range of creatine kinase in a blood sample from Salmonids is any amount of creatine kinase between 3072 and 10437.6 U/L (green), the high abnormal range is any amount of creatine kinase between 10437.6 and 14120.4 U/L (amber), the low abnormal range is any amount of creatine kinase less than 3072 U/L (amber), the high unhealthy range is any amount of creatine kinase between 14120.4 and 17803.2 U/L (red).

In some embodiments a representative healthy reference range of iron in a blood sample from Salmonids is any amount of iron between 9.4 and 17.40 μmol/L (green), the high abnormal range is any amount of iron between 17.40 and 21.40 μmol/L (amber), the low abnormal range is any amount of iron between 5.40 and 9.40 μmol/L (amber), the low unhealthy range is any amount between 0 and 5.4 μmol/L (red) and the high unhealthy range is any amount of iron between 21.40 and 25.40 μmol/L (red).

In some embodiments a representative healthy reference range of lactate in a blood sample from Salmonids is any amount of lactate between 2 and 3 mmol/L (green), the high abnormal range is any amount of lactate between 3 and 3.5 mmol/L (amber), the low abnormal range is any amount of lactate between 1.5 and 2 mmol/L (amber), the low unhealthy range is any amount of lactate below 1.5 mmol/L (red) and the high unhealthy range is any amount of lactate between 3.5 and 4 mmol/L (red).

In some embodiments a representative healthy reference range of lactate dehydrogenase in a blood sample from Salmonids is any amount of lactate dehydrogenase between 322.9 and 1285.5 U/L (green), the high abnormal range is any amount of lactate dehydrogenase between 1285.5 and 1766.8 U/L (amber), the low abnormal range is any amount of lactate dehydrogenase below 322.9 U/L (amber), and the high unhealthy range is any amount of lactate dehydrogenase between 1766.8 and 2248.1 U/L(red).

In some embodiments a representative healthy reference range of lipase in a blood sample from Salmonids is any amount of lipase between 6.1 and 7.7 U/L (green), the high abnormal range is any amount of lipase between 7.7 and 8.5 U/L (amber), the low abnormal range is any amount of lipase between 5.3 and 6.1 U/L (amber), the low unhealthy range is any amount of lipase below 5.3 U/L (red) and the high unhealthy range is any amount of lipase between 8.5 and 9.3 U/L (red).

In some embodiments a representative healthy reference range of magnesium in a blood sample from Salmonids is any amount of magnesium between 1 and 1.2 mmol/L (green), the high abnormal range is any amount of magnesium between 1.2 and 1.3 mmol/L (amber), the low abnormal range is any amount of magnesium between 0.9 and 1 mmol/L (amber), the low unhealthy range is any amount of magnesium below 0.9 mmol/L (red) and the high unhealthy range is any amount of magnesium between 1.3 and 1.4 mmol/L (red).

In some embodiments a representative healthy reference range of phosphate in a blood sample from Salmonids is any amount of phosphate between 3.3 and 4.5 mmol/L (green), the high abnormal range is any amount of phosphate between 4.5 and 5.1 mmol/L (amber), the low abnormal range is any amount of phosphate between 2.7 and 3.3 mmol/L (amber), the low unhealthy range is any amount of phosphate below 2.7 mmol/L (red) and the high unhealthy range is any amount of phosphate between 5.1 and 5.7 mmol/L (red).

In some embodiments a representative healthy reference range of sodium in a blood sample from Salmonids is any amount of sodium between 150.7 and 163.5 mmol/L (green), the high abnormal range is any amount of sodium between 163.5 and 169.9 mmol/L (amber), the low abnormal range is any amount of sodium between 144.3 and 150.7 mmol/L (amber), the low unhealthy range is any amount of sodium between 137.9 and 144.3 mmol/L (red) and the high unhealthy range is any amount of sodium between 169.9 and 176.3 mmol/L (red).

In some embodiments a representative healthy reference range of potassium in a blood sample from Salmonids is any amount of potassium between 0.8 and 1.2 mmol/L (green), the high abnormal range is any amount of potassium between 1.2 and 1.4 mmol/L (amber), the low abnormal range is any amount of potassium between 0.6 and 0.8 mmol/L (amber), the low unhealthy range is any amount of potassium less than 0.6 mmol/L (red) and the high unhealthy range is any amount of potassium between 1.4 and 1.6 mmol/L (red).

In some embodiments a representative healthy reference range of chloride in a blood sample from Salmonids is any amount of chloride between 117.4 and 131.2 mmol/L (green), the high abnormal range is any amount of chloride between 131.2 and 138.1 mmol/L (amber), the low abnormal range is any amount of chloride between 110.5 and 117.4 mmol/L (amber), the low unhealthy range is any amount of chloride between 103.6 and 110.5 mmol/L (red) and the high unhealthy range is any amount of chloride between 138.1 and 145 mmol/L (red).

In some embodiments a representative healthy reference range of zinc in a blood sample from Salmonids is any amount of zinc between 189.1 and 334.7 μmol/L (green), the high abnormal range is any amount of zinc between 334.7 and 407.5 μmol/L (amber), the low abnormal range is any amount of zinc between 116.3 and 189.1 μmol/L (amber), the low unhealthy range is any amount of zinc between 43.5 and 116.3 μmol/L (red) and the high unhealthy range is any amount of zinc between 407.5 and 480.3 μmol/L (red).

In some embodiments a representative healthy reference range of creatine kinase-myocardial band in a blood sample from Salmonids is any amount of creatine kinase-myocardial band between 5080.7 and 17200.9 U/L (green), the low abnormal range is any amount of creatine kinase-myocardial band less than 5080.7 U/L (amber), the high abnormal range is any amount of creatine kinase-myocardial band between 17200.9 and 23261.0 U/L (amber), and the high unhealthy range is any amount of creatine kinase-myocardial band between 23261.0 and 29321.1 U/L (red).

In some embodiments a representative healthy reference range of glucose in a blood sample from Salmonids is any amount of glucose between 4.3 and 6.7 mmol/L (green), the low abnormal range is any amount of glucose between 3.1 and 4.3 mmol/L (amber), the high abnormal range is any amount of glucose between 6.7 and 7.9 mmol/L (amber), the low unhealthy range is any amount of glucose between 1.9 and 3.1 mmol/L (red) and the high unhealthy range is any amount of glucose between 7.9 and 9.1 mmol/L (red).

In some embodiments of the present invention, the healthy range, abnormal range and unhealthy range is determined by the concentration of each biomarker as indicated in Table 4. Where the value of “0” is indicated in the table, this is representative of a below threshold reading i.e., below the limit of detection of the assay. Suitably, where a value of “0” is attributed, the analyte is considered to fall in the next analyte reference range.

The skilled person will understand that the analyte reference ranges above apply to any aspect of the present invention as described herein. The analyte reference ranges as described above and in Table 4 are indicative of representative ranges in a blood sample from Salmonids. The skilled person can suitably apply equivalent ranges to other species such as cichlidae, carp or acipenseridae and shellfish or for other types of sample. Suitably, in some embodiments, a healthy range for each analyte is determined by the background level of the reference profile as described above. Suitably, in some embodiments the background level may refer to the amount of each analyte in a representative healthy fish or the average amount of each analyte determined from a population of healthy fish. The analyte reference range can in some embodiments be determined as the mean of the background level of a healthy fish or a population of fish ±1 standard deviation (SD), with ±1 to 2 SDs representing an abnormal range indicative of a degeneration in fish health, and ±more than 2 SDs representing unhealthy samples.

Suitably, in some embodiments, a change in the health status of the population of fish may refer to improvement or a worsening in health status. Worsening may refer to an increase or decrease in the amount of at least one analyte in the sample as compared to the reference profile. Worsening may refer to the at least one analyte entering the abnormal range or entering the unhealthy range. Improving may refer to an increase or decrease in the amount of analyte in the sample. Improving may refer to the amount of the analyte entering the abnormal range from the unhealthy range or entering the normal range from the abnormal range or unhealthy range.

The skilled person will understand that a greater number of analytes that are classified as abnormal and/or unhealthy is more strongly indicative of an unhealthy fish or an unhealthy population of fish. Similarly, a greater number of analytes that are classified as abnormal and/or unhealthy is more strongly indicative that the fish or a population of fish have a disease as described herein.

Suitably, in one embodiment, at least one, at least two, at least three, at least four, at least five, at least six, at least seven, at least eight, at least nine, at least ten, at least eleven, at least twelve, at least thirteen, at least fourteen, at fifteen, at least sixteen, at least seventeen, at least eighteen of the analytes described herein being classified in the abnormal and/or unhealthy analyte reference ranges indicates an unhealthy fish.

It will be understood by the skilled person that it may be beneficial in some instances to produce a population welfare score for a population of fish. The skilled person will understand that predicting a population welfare score for a population of fish has numerous advantages for fish farm management, such as making site wide animal husbandry decisions, identifying a particular enclosure (e.g., cage, pen or tank) on a site suffering from ill health and allowing farm managers to monitor the health of their fish stock.

Suitably, in a further aspect there is provided a method for determining a population welfare score for a population of fish, wherein the method comprises:
(a) determining the health status of a plurality of fish from a population of fish according to the first aspect;
(b) classifying each sample obtained from a plurality of fish from the population of fish into one of a plurality of health level categories; and
(c) providing a score corresponding to the proportion of fish in each health level category wherein the score is indicative of the overall population welfare.

Accordingly, the population welfare score for a population of fish represents the overall health of the population based on the health level category of a plurality of fish from the population.

In some embodiments the population welfare score comprises an indication of the ratio of fish in each of the plurality of health level categories. In some embodiments the population welfare score comprises an arithmetical manipulation of the number of fish in each of the plurality of health level categories, e.g., the number of healthy fish divided by the number of unhealthy fish.

It will be appreciated that various health level categories can be defined in various ways by the skilled person. In some exemplary embodiments, the health level categories may be healthy or unhealthy. In some exemplary embodiments, the health level categories may be good health, weak health or critical health. In some embodiments, the good health category defines the highest level of the healthiness of a fish. In some embodiments, the weak health category defines an acceptable level of the healthiness of a fish. In some embodiments, the critical health category defines a sick or unhealthy fish.

In some embodiments a good or healthy population welfare score is indicated by a high proportion or high ratio of fish in the healthy or good health category. In some embodiments, an intermediate or weak health population welfare score is indicated by a high proportion or high ratio of fish in the weak health category. In some embodiments, a poor health population welfare score is indicated by a high ratio or proportion of fish in the unhealthy or critical health category.

In some embodiments the population welfare score is determined by calculating the ratio of fish from a population of fish classified as good, weak or critical health to provide a population welfare score. The skilled person will understand that the greater the number of samples categorised in the healthy or good health category will provide a healthy population welfare score. In contrast, where most samples belong to the weak health category then a weak health population welfare score is awarded to the population of fish. Where the majority of samples belong to the critical health category, then a critical health population welfare score is awarded to the population of fish. Suitably, in some embodiments the population welfare score is the cumulative health scores of individual fish from a population of fish.

### Health Status Monitoring

The skilled person will understand that it may be desirable to perform the method of the first or second aspect to first determine the health status of a population of fish and then continue to monitor the health status of the population of fish.

Thus, in some embodiments the methods of the first or second aspects can be repeated over a period of time to determine changes in the health status or population welfare score. This allows for the early identification of changes in the health of a population of fish, thus allowing for early intervention by a fish farmer.

According to a third aspect of the invention, there is provided a method of monitoring the health status of a population of fish over time, wherein the method comprises:
(a) analysing a first sample collected from at least one fish of the population of fish to determine the amount of at least one analyte selected from the group consisting of: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium present in the sample to determine a first test profile; and
(b) comparing the first test profile with a reference profile, wherein a difference in the first test profile as compared to the reference profile indicates the health status of the population of fish, and
(c) analysing at least a second sample collected from at least one fish of the population of fish to determine the amount of the same at least one analyte present in the at least second sample to determine a second test profile; and
(d) comparing the second test profile with a reference profile; wherein a difference in the second test profile as compared to a reference profile indicates the health status of the population of fish, and comparing the health status indicated by the first test profile with the health status indicated by the second test profile.

Typically the first and second samples are obtained at different times. Accordingly, the health status of a population of fish can be monitored over time.

Optionally monitoring methods as described herein can comprise analysing one or more further samples.

In an alternative method according to the third aspect, the second test profile can be compared with the first test profile to determine if fish health has changed over time.

Accordingly, there is provided a method of monitoring the health status of a population of fish over time, wherein the method comprises:
(a) analysing a first sample collected from at least one fish of a population of fish to determine the amount of at least one analyte selected from the group consisting of: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium present in the sample to determine a first test profile; and
(b) analysing at least a second sample collected from at least one fish of the population of fish to determine the amount of the same at least one analyte present in the at least second sample to determine a second test profile;
(c) comparing the second test profile with the first test profile;
wherein a difference second test profile as compared to the first test profile indicates the health status of the population of fish has changed.

Optionally, either the first of second test profiles, or both the first and second test profiles, can be compared to a reference profile to determine the significance of any change in the amount of the at least one analyte.

In some embodiments according to any aspect described herein, the method may be performed prior to observing physical or behavioural characteristics of a condition or disease. In one embodiment, the health status of the fish provides an early indication of the condition or disease prior to observing physical or behavioural characteristic of the condition of disease.

Physical or behavioural characteristics of a condition or disease may refer to, but are not limited to, loss of appetite, weakness, moribund, loss of balance or buoyancy control, changes to swimming patterns, separation from the group, gasping or mouthing for air, changes to respiratory rate or laboured breathing and/or clamped fins.

In some embodiments, monitoring health status of a population of fish comprises repeated monitoring. In some embodiments, repeated monitoring can be regular or irregular monitoring. Regular monitoring may refer to monitoring the health status of the population of fish at routine intervals such as daily, weekly, monthly, annually, etc. Irregular monitoring may refer to monitoring the health status of the population of fish on an ad hoc basis. Suitably, in some embodiments, continuous health status monitoring of the fish population comprises performing the method of the first aspect, second aspect or third aspect of the invention every week, every two weeks, every three weeks, every four weeks, ever five weeks, every six weeks. The first, second or third aspect of the invention may be performed monthly or annually. Suitably, it may be desirable to perform the first, second or third aspect of the invention in spring months and/or in summer months.

The frequency of monitoring may vary depending on the temperature of water as fish in warmer waters (e.g., water temperature 10 °C or higher) are more susceptible to pests and disease. Suitably, in some embodiments more frequent monitoring is required. In some embodiments, in warmer water the monitoring the health status of a population of fish is more frequent. Suitably, the population of fish may be monitored daily, bi-weekly, weekly and/or bi-monthly. In colder water environments, the population of fish may be monitored less frequently (e.g., water temperature less than 10 °C). Suitably, the population of fish may be monitored weekly, bi-monthly, monthly, quarterly or annually.

Suitably, in some embodiments, monitoring health status of a population of fish is repeated monitoring by obtaining at least one sample from a population of fish samples every 4 weeks during the colder water months and/or every 2 weeks in warmer water months.

More frequent monitoring may be required in waters susceptible to plankton blooms such as algal blooms or micro-jellyfish blooms. Such waters include but are not limited to warmer waters (e.g., water temperature 10 °C or higher) and nutrient dense waters.

It will be understood that plankton blooms may result in a drop in the water oxygen concentration. Low oxygen concentrations can increase fish mortality rates and the plankton themselves can result in gill impact and potentially disease. Suitably, in nutrient dense waters, the monitoring the health status of a population of fish is more frequent. Suitably, the population of fish may be monitored daily, bi-weekly, weekly and/or bi-monthly.

The skilled person will understand that nutrient dense waters include areas of water where nutrients are introduced into the water in excess of normal environmental levels. Examples include, the excess phosphate and excess nitrogen as a result of fertilisers and agriculture and sewage run off. For example, the natural levels of phosphorous usually range from 0.005 to 0.05 mg/L. However, the natural background levels of total phosphorus are generally less than 0.03 mg/L. Nutrient dense waters may have greater than 0.03 mg/L of phosphorous. Nitrogen levels in water may be calculated by measuring the concentration of dissolved inorganic nitrogen (DIN). The concentration of DIN is considered high if it is more than 50% above a background concentration taken as 10 μM in seawater and 42 μM in river water. Enrichment with nitrogen is therefore indicated by concentrations of DIN greater than 15 μM in offshore waters and 18 μM in regions of freshwater influence such as coastal waters. In estuaries, nitrogen enrichment is indicated by concentrations greater than 30 μM measured at a salinity of 25, which represents an approximate mix of 1:2.5 parts freshwater:seawater.

Warmer waters are typically considered to be waters with a temperature of 10 °C or higher. Colder waters are typically considered to be waters with a temperature of less than 10 °C.

In some embodiments, according to any aspect of the invention, the method comprises analysing a sample from at least one fish from a population of fish. In another embodiment, the method comprises analysing a sample from an individual fish. Suitably, the sample may refer to the first sample and/or the at least one later sample. Preferably, in some embodiments more than one sample is obtained from the population of fish. Suitably, the method of the first, second and/or third aspect of the invention comprises analysing a plurality of samples from a plurality of fish from a population of fish. Suitably, a plurality of fish may include at least one, least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand fish. Suitably, a plurality of samples may include at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand, at least ten thousand samples obtained from one or more fish from a population of fish.

In some embodiments, monitoring the health status of a population of fish comprises analysing a sample from at least one fish from a population of fish. Preferably, a sample is obtained from a plurality of fish. In one embodiment, the method of any aspect of the invention samples are analysed from a population of fish from at least one enclosure, at least two enclosures, at least three enclosures, at least four enclosures, at least five enclosures, at least ten enclosures. In some embodiments, a population of fish includes fish housed in different enclosures.

In one embodiment according to the method of any aspect of the invention, at least ten fish from a population of fish from at least 3 enclosures are sampled every four weeks during the colder water months and every two weeks in warmer water months. Suitably, in some embodiments, during the colder water months at least 30 fish are sampled every four weeks. Suitably, in some embodiments, during the warmer water months, at least 30 fish are sampled every two weeks.

It is obvious to the skilled person that colder and warmer water months are variable across geographical locations and are variable year on year. In some embodiments, colder water months span November to April and warmer water months span May to October.

### Diagnosis

The present invention further relates to diagnosing a fish with a condition or disease. In some embodiments the invention relates to diagnosing at least one fish in a population of fish with a condition or a disease. The present invention also provides diagnosing a population of fish with a condition or a disease. As used herein, diagnosis or diagnosing may refer to determining, identifying or classifying a condition or disease in at least one fish or in a population of fish. Suitably, the skilled person would understand that diagnosing a population of fish with a condition or disease may comprise determining the incidence or the prevalence of a condition or disease in the population of fish. Suitably, in some embodiments of the invention as described herein, diagnosing a population of fish with a condition or a disease includes identifying an outbreak of a condition or a disease in the population of fish. Diagnosing includes identifying any disease or condition in a fish or in a population of fish. This may include further classification of disease type, such as, without limitation, pancreas disease, cardiomyopathy syndrome, gill issues, gill issues associated with an environmental challenge (e.g., plankton blooms), heart and skeletal muscle inflammation and/or bacterial infection. In some embodiments, a fish or a population of fish may be diagnosed a moribund. In such embodiments, moribund is the terminal phase of any condition or disease as described herein. The term ‘health challenge’ may be used interchangeably with the terms ‘condition’ or ‘disease’.

Accordingly, a fourth aspect of the present invention provides a method of diagnosing a fish or a population of fish with a condition or a disease, the method comprising:
(a) analysing a first sample collected from at least one fish of a population of fish to determine the amount of at least one analyte selected from: chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium present in the sample to determine a test profile;
(b) comparing the amount of the at least one analyte present in the test profile with a reference profile;
wherein a difference in the amount of the at least one analyte in the test profile as compared to the reference profile indicates the at least one fish has a condition or a disease.

Preferably the reference profile is a reference profile of a healthy fish. Thus, a deviation from the reference profile is indicative of the presence of a condition or disease.

Typically the method comprises analysing a plurality of samples obtained from a plurality of fish in the population to obtain a plurality of test profiles. From this the incidence or the prevalence of a condition or disease in the population of fish can conveniently be determined.

Suitably, in some embodiments, a decrease in the amount of any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more or eighteen analytes selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium in the test profile compared to the reference profile indicates that the fish or the population of fish has a condition or a disease.

Suitably, in some embodiments, an increase in the amount of any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more or eighteen of analytes selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium, in the test profile compared to the reference profile indicates the fish or the population of fish has a condition or a disease.

Suitably, in some embodiments, an increase and/or a decrease in the amount of any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more or eighteen analytes selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium in the test profile compared to the reference profile indicates that the fish or the population of fish has a condition or a disease.

Suitably, in some embodiments, when the amount of any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more or eighteen analytes selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium in the test profile are in the abnormal and/or unhealthy analyte reference range as described above, this indicates that the fish or the population of fish has a condition or a disease.

In some embodiments, an increase in the amount of alanine aminotransferase, aspartate aminotransferase, creatine kinase, creatine kinase-myocardial band, lactate, lactate dehydrogenase, magnesium, phosphate, sodium, potassium and chloride and a decrease in the amount of albumin, calcium, total cholesterol, glucose, iron and zinc indicates that the fish or the population of fish has a condition or a disease. In some embodiments, such a fish or population of fish may be referred to as an unhealthy fish.

It will be understood by the skilled person that the greater the number of analytes that are classified as abnormal and/or unhealthy when compared to the healthy reference profile the more strongly indicative that the fish or the population of fish have a condition or disease as described herein.

In some embodiments, the condition or disease is any one or more of pancreas disease, cardiomyopathy syndrome, gill issues, gill issues associated with an environmental challenge, heart and skeletal muscle inflammation and/or bacterial infection. In another embodiment, the condition or disease is moribund.

Suitably, in some embodiments, where the amount of any one or more, any two or more, any three or more, any four or more, any five or more or any six or more, any seven or more, any eight or more, any nine or more or any ten analytes selected from: creatinine kinase, creatine kinase-myocardial band, lactate dehydrogenase, alanine aminotransferase, lipase, total cholesterol, glucose, zinc, phosphate or iron in the test profile are different compared to the reference profile this indicates that the fish has pancreas disease. Suitably, in some embodiments, where the amount of any one or more, any two or more, any three or more, any four or more, any five or more or any six or more, any seven or more, any eight or more, any nine or more or any ten analytes selected from: creatinine kinase, creatine kinase-myocardial band, lactate dehydrogenase, alanine aminotransferase, lipase, total cholesterol, glucose, zinc, phosphate or iron are in the abnormal and/or unhealthy analyte reference range, this indicates that the fish or the population of fish has pancreas disease.

Suitably, in some embodiments, where the amount of any one or more, any two or more, any three or more, any four or more, any five or more or any six or more, any seven or more, any eight or more, any nine or more or ten or more, eleven or more, twelve or more, thirteen or more, fourteen or more, fifteen or more, sixteen or more, seventeen or more, or eighteen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, total cholesterol, calcium and glucose in the test profile are different compared to the reference profile this indicates that the fish has pancreas disease.

Suitably, in one embodiment, an increase in the amount of creatinine kinase, creatine kinase-myocardial band, aspartate aminotransferase and lipase and a decrease in the amount of total cholesterol, glucose, zinc, phosphate and iron when compared to the reference profile indicates that the fish or the population of fish has pancreas disease.

Suitably, in one embodiment, an increase in the amount of creatinine kinase, creatine kinase-myocardial band, aspartate aminotransferase and lipase and a decrease in the amount of total cholesterol, glucose, zinc and iron when compared to the reference profile indicates that the fish or the population of fish has pancreas disease.

Suitably, in one embodiment, an increase in the amount of creatinine kinase, creatine kinase-myocardial band, lactate dehydrogenase, alanine aminotransferase, aspartate aminotransferase and lipase and a decrease in the amount of total cholesterol, glucose, zinc phosphate and iron when compared to the reference profile indicates that the fish or the population of fish has pancreas disease.

Suitably, in one embodiment, an increase in the amount of creatinine kinase, creatine kinase-myocardial band, lactate dehydrogenase, alanine aminotransferase, aspartate aminotransferase and lipase and a decrease in the amount of total cholesterol, glucose, zinc and iron when compared to the reference profile indicates that the fish or the population of fish has pancreas disease.

In one embodiment, an increase in the amount of creatinine kinase, creatine kinase-myocardial band, lactate dehydrogenase, lactate, alanine aminotransferase, aspartate aminotransferase, sodium, potassium, chloride and lipase and a decrease in the amount of total cholesterol, glucose, zinc and iron when compared to the reference profile indicates that the fish or the population of fish has pancreas disease.

In one embodiment, an increase in the amount of alanine aminotransferase, aspartate aminotransferase, creatine kinase, creatine kinase-myocardial band, lactate, lactate dehydrogenase, lipase, magnesium, sodium, potassium and chloride and a decrease in albumin, calcium, total cholesterol, glucose, iron, phosphate and zinc when compared to the reference profile indicates that the fish or the population of fish has pancreas disease.

In another embodiment, where the amount of any one or more, any two or more, any three or more, any four or more, any five or more or any six or more, any seven or more, any eight or more, any nine or more, any ten or more, any eleven or more, any twelve or more, any thirteen or more or any fourteen analytes selected from: creatine kinase-myocardial band, lactate, creatine kinase, aspartate aminotransferase, magnesium, phosphate, lactate, sodium, potassium chloride, albumin, zinc, iron or total cholesterol in the test profile are different compared to the reference profile, this indicates that the fish or the population of fish has cardiomyopathy syndrome.

In another embodiment, where the amount of any one or more, any two or more, any three or more, any four or more, any five or more or any six or more, any seven or more, any eight or more, any nine or more, any ten or more, any eleven or more, any twelve or more, any thirteen or more. any fourteen or more or any fifteen or more analytes selected from: creatine kinase-myocardial band, lactate, creatine kinase, alanine aminotransferase, aspartate aminotransferase, magnesium, phosphate, lactate, sodium, potassium chloride, albumin, zinc, iron or total cholesterol in the test profile are different compared to the reference profile, this indicates that the fish or the population of fish has cardiomyopathy syndrome. Suitably, in some embodiments, where the amount of any one or more, any two or more, any three or more, any four or more, any five or more or any six or more, any seven or more, any eight or more, any nine or more, any ten or more, any eleven or more, any twelve or more, any thirteen or more or any fourteen analytes selected from: creatine kinase-myocardial band, lactate, creatine kinase, aspartate aminotransferase, magnesium, phosphate, lactate, sodium, potassium chloride, albumin, zinc, iron or total cholesterol are in the abnormal and/or unhealthy analyte reference range, this indicates that the fish or the population of fish has cardiomyopathy syndrome.

Suitably, in some embodiments, where the amount of any one or more, any two or more, any three or more, any four or more, any five or more or any six or more, any seven or more, any eight or more, any nine or more, any ten or more, any eleven or more, any twelve or more, any thirteen or more, any fourteen or more or any fifteen analytes selected from: creatine kinase-myocardial band, lactate, creatine kinase, alanine aminotransferase, aspartate aminotransferase, magnesium, phosphate, lactate, sodium, potassium chloride, albumin, zinc, iron or total cholesterol are in the abnormal and/or unhealthy analyte reference range, this indicates that the fish or the population of fish has cardiomyopathy syndrome.

Suitably in some embodiments, an increase in the amount of creatine kinase-myocardial band, lactate dehydrogenase, creatine kinase, aspartate aminotransferase, magnesium, phosphate, lactate, sodium, potassium and chloride and a decrease in the amount of albumin, zinc, iron and total cholesterol when compared to the reference profile indicates the fish or the population of fish has cardiomyopathy syndrome.

Suitably in some embodiments, an increase in the amount of creatine kinase-myocardial band, lactate dehydrogenase, creatine kinase, aspartate aminotransferase, magnesium, phosphate, lactate, sodium, potassium and chloride and a decrease in the amount of albumin, zinc, iron and total cholesterol and no change in the amount of calcium, lactate dehydrogenase and lipase when compared to the reference profile indicates the fish or the population of fish has cardiomyopathy syndrome.

In one embodiment, an increase in the amount of lactate, magnesium, phosphate, sodium, potassium and chloride and a decrease in the amount of albumin, total cholesterol, glucose, iron and zinc when compared to the reference profile indicates the fish or the population of fish has cardiomyopathy syndrome.

In one embodiment, an increase in the amount of lactate, sodium, potassium and chloride and a decrease in the amount of albumin and zinc when compared to the reference profile indicates that a fish or a population of fish has cardiomyopathy syndrome.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more or any seventeen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, total cholesterol and glucose in the test profile are different compared to the reference profile, this indicates that the fish or the population of fish has gill issues.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more, any seventeen or more or eighteen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, total cholesterol, calcium and glucose in the test profile are different compared to the reference profile, this indicates that the fish or the population of fish has gill issues.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more or any seventeen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, total cholesterol and glucose are in the abnormal and/or unhealthy analyte reference range, this indicates that the fish or the population of fish has gill issues.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more, any seventeen or more, or eighteen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, calcium, total cholesterol and glucose are in the abnormal and/or unhealthy analyte reference range, this indicates that the fish or the population of fish has gill issues.

Suitably, in one embodiment, an increase in the amount of magnesium, phosphate, lactate, sodium, potassium and chloride and a decrease in the amount of glucose when compared to the reference profile indicates that the fish or the population of fish has gill issues.

Suitably, in one embodiment, an increase in the amount of creatine kinase-myocardial band, lactate dehydrogenase, lactate, alanine aminotransferase, creatine kinase, aspartate aminotransferase, magnesium, phosphate, lactate, sodium, potassium and chloride and a decrease in the amount of albumin, zinc, iron, total cholesterol and glucose when compared to the reference profile indicates that the fish or the population of fish has gill issues.

Suitably, in one embodiment, an increase in the amount of alanine aminotransferase, aspartate aminotransferase, calcium, creatine kinase, creatine kinase-myocardial band, lactate, lactate dehydrogenase, magnesium, phosphate, sodium, potassium and chloride, and a decrease in the amount of albumin, total cholesterol, glucose, iron and zinc when compared to the reference profile indicates that the fish or the population of fish has gill issues.

Suitably, in one embodiment, an increase in the amount of alanine aminotransferase, aspartate aminotransferase, creatine kinase, creatine kinase-myocardial band, lactate, lactate dehydrogenase, magnesium, phosphate, sodium, potassium and chloride, and a decrease in the amount of calcium, albumin, total cholesterol, glucose, iron and zinc when compared to the reference profile indicates that the fish or the population of fish has gill issues.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more or any seventeen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, total cholesterol and glucose in the test profile are different compared to the reference profile, this indicates that the fish or the population of fish has gill issues associated with an environmental challenge.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more, any seventeen or more, or eighteen analytes selected from: creatine kinase-myocardial band, lactate dehydrogenase, lactate, alanine aminotransferase, creatine kinase, aspartate aminotransferase, magnesium, phosphate, lactate, sodium, potassium, chloride, albumin, zinc, iron, calcium, total cholesterol and glucose are in the abnormal and/or unhealthy analyte reference range, this indicates that the fish or the population of fish has gill issues associated with an environmental challenge.

Suitably, in one embodiment, an increase in the amount of magnesium, phosphate, lactate, sodium, potassium and chloride and a decrease in the amount of glucose when compared to the reference profile indicates that the fish or the population of fish has gill issues associated with an environmental challenge.

Suitably, in one embodiment, an increase in the amount of alanine aminotransferase, aspartate aminotransferase, creatine kinase, creatine kinase-myocardial band, lactate, lactate dehydrogenase, magnesium, phosphate, sodium, potassium and chloride, and a decrease in the amount of calcium, albumin, total cholesterol, glucose, iron and zinc when compared to the reference profile indicates that the fish or the population of fish has gill issues associated with an environmental challenge.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more or any seventeen analytes selected from: creatine kinase-myocardial band, lactate dehydrogenase, alanine aminotransferase, creatine kinase, aspartate aminotransferase, magnesium, phosphate, lactate, sodium, potassium, chloride, albumin, calcium, zinc, iron, total cholesterol and glucose in the test profile are different compared to the reference profile, this indicates that the fish has heart and skeletal muscles inflammation. Suitably, in another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more or any seventeen analytes selected from: creatine kinase-myocardial band, lactate dehydrogenase, alanine aminotransferase, creatine kinase, aspartate aminotransferase, magnesium, phosphate, lactate, sodium, potassium, chloride, albumin, calcium, zinc, iron, total cholesterol and glucose are in the abnormal and/or unhealthy analyte reference range, this indicates that the fish or the population of fish has heart and skeletal muscle inflammation.

Suitably, in one embodiment, an increase in the amount of creatine kinase-myocardial band, lactate dehydrogenase, creatine kinase and aspartate aminotransferase and a decrease in albumin, zinc and iron when compared to the reference profile indicates the fish or the population of fish has heart and skeletal muscle inflammation.

Suitably, in one embodiment, an increase in the amount of creatine kinase-myocardial band, lactate dehydrogenase, alanine aminotransferase, creatine kinase, aspartate aminotransferase, magnesium, phosphate, lactate, sodium, potassium and chloride and a decrease in albumin, calcium, zinc, iron, total cholesterol and glucose when compared to the reference profile indicates the fish or the population of fish has heart and skeletal muscle inflammation.

In some embodiments, an increase in the amount of alanine aminotransferase, aspartate aminotransferase, creatine kinase, creatine kinase-myocardial band, lactate, lactate dehydrogenase, magnesium, phosphate, sodium, potassium, chloride, a decrease in the amount of albumin, calcium, total cholesterol, glucose, iron and zinc and no change in the amount of lipase when compared to the reference profile indicates the fish or the population of fish has heart and skeletal muscle inflammation.

In yet another embodiment, where the amount of any one or more, any two or more, any three or more, any four or more, any five or more or any six or more, any seven or more, any eight or more, any nine or more, any ten or more, any eleven or more, any twelve or more or any thirteen analytes selected from: lactate dehydrogenase, lactate, magnesium, phosphate, sodium, potassium, chloride, albumin, calcium, total cholesterol, glucose, iron and zinc in the test profile are different compared to the reference profile, this indicates that the fish or the population of fish has a bacterial infection. In another embodiment, where the amount of any one or more, any two or more, any three or more, any four or more, any five or more or any six or more, any seven or more, any eight or more, any nine or more, any ten or more, any eleven or more, any twelve or more or any thirteen of: lactate dehydrogenase, lactate, magnesium, phosphate, sodium, potassium, chloride, albumin, calcium, total cholesterol, glucose, iron and zinc are in the abnormal and/or unhealthy analyte reference range, this indicates that the fish or the population of fish has a bacterial infection.

In another embodiment, where the amount of any one or more, any two or more, any three or more, any four or more, any five or more or any six or more, any seven or more, any eight or more, any nine or more, any ten or more, any eleven or more, any twelve or more, any thirteen or more, any fourteen or more, any fifteen or more, any sixteen or more, any seventeen or more or any eighteen of: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium are in the abnormal and/or unhealthy analyte reference range, this indicates that the fish or the population of fish has a bacterial infection.

Suitably, in one embodiment, an increase in the amount of lactate dehydrogenase, lactate, magnesium, phosphate, sodium, potassium and chloride and a decrease in the amount of albumin, calcium, total cholesterol, glucose, iron and zinc when compared to the reference profile indicates that the fish or the population of fish has a bacterial infection.

In one embodiment, an increase in the amount of lactate dehydrogenase and lactate and a decrease in the amount of albumin, total cholesterol and iron when compared to the reference profile indicates the fish or the population of fish has a bacterial infection.

In one embodiment, an increase in the amount of alanine aminotransferase, aspartate aminotransferase, creatine kinase, creatine kinase-myocardial band, lactate, lactate dehydrogenase, magnesium, phosphate, sodium, potassium and chloride and a decrease in albumin, calcium, total cholesterol, lipase and zinc when compared to the reference profile indicates the fish or the population of fish has a bacterial infection.

In one embodiment, an increase in the amount of alanine aminotransferase, aspartate aminotransferase, creatine kinase, creatine kinase-myocardial band, lactate, lactate dehydrogenase, magnesium, phosphate, sodium, potassium and chloride and a decrease in albumin, calcium, total cholesterol, lipase, glucose, iron and zinc when compared to the reference profile indicates the fish or the population of fish has a bacterial infection.

In another embodiment, the terminal phase of any condition or disease as described herein may be determined when the amount of any one or more, any two or more, any three or more, any four or more, any five or more, any six or more, any seven or more, any eight or more, any nine or more or any ten analytes selected from: lactate, magnesium, phosphate, sodium, potassium, chloride, albumin, total cholesterol, iron and zinc are in the unhealthy analyte reference range as described above. In some embodiments, the terminal phase is described as moribund.

In one embodiment, an increase in the amount of lactate, magnesium, phosphate, sodium, potassium and chloride and a decrease in albumin, total cholesterol, iron and zinc when compared to reference profile indicates the fish or the population of fish are moribund.

According to a fifth aspect, there is provided herein a method for monitoring progression of a condition or disease in a population of fish, comprising the steps of:
(a) analysing one or more first samples collected from at least one fish of the population of fish at a first time point to determine the amount of at least one analyte, the analyte preferably being selected from the group consisting of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium to determine a first test profile, and
(b) analysing at least one later sample collected from at least one fish of the population of fish at a at least one later time point to determine the amount of the same at least one analyte present in the least one later sample to determine at least one later test profile;
   determining from a comparison between the first test profile and the second test profile the progression of the condition or the disease.

In some embodiments the method comprises comparing the first test profile with a reference profile. In some embodiments the method comprises comparing the at least one later test profile with a reference profile. In some embodiments the first test profile and the at least one later test profile is compared with a reference profile. In some embodiments the method comprises comparing the first test profile with at least one later test profile and determining a change in the test profiles.

Progression as used herein may refer to improvement or a worsening in the condition or disease. Worsening may be indicated by an increase or decrease in the amount of analyte in the sample as compared to the reference profile. Worsening may be indicated by one or more analytes entering the abnormal analyte reference range as described herein or entering the unhealthy analyte reference range as described herein. In some embodiments worsening may be indicated by one or more analytes entering the abnormal range defined as ±1 to 2 SDs from background/reference levels or entering the unhealthy range defined as ±greater than 2 SDs from background/reference levels. Improving may be indicated by an increase or decrease in the amount of one or more analytes in the sample. Improving may be indicated by one or more analytes entering the abnormal analyte reference range from the unhealthy analyte reference range as described herein or entering the healthy analyte reference range as described herein. In some embodiments, improving may be indicated by the amount of one or more analytes entering the abnormal range as defined as mean±1 to 2 SDs from the unhealthy range as defined as mean±greater than 2 SDs or entering the normal range as defined as mean±1SD from the abnormal range or unhealthy range.

Current diagnostic testing is typically sporadic, only occurring when a specific health challenge arises. Advantageously, the present invention provides methods for both diagnostic testing before any conventional physiological or behavioural aspects of a condition or disease are observed and before the population of fish display symptoms of a condition or disease. This has the advantage over current methods available in the field that conditions or diseases can be diagnosed earlier. Suitably, in some aspects a sample is analysed for one or more analytes comprised in Table 3. In one embodiment, the one or more analytes analysed in the sample is selected based on the observed condition or suspected condition or disease affecting one or more fish, or the one or more analytes analysed may be determined by other factors such as seasonal risks or concern about transmission from a neighbouring site. Suitably, in some embodiments, the symptoms of a particular condition or disease as displayed by fish may dictate the panel of biomarkers selected for analysis.

In any of the aspects and embodiments discussed herein, the amount of the at least one analyte in the test profile is suitably compared to the reference profile as described above. This allows the health condition to be determined, a welfare score to be determined or one or more diseases or conditions to be diagnosed in one or more fish or in a population of fish.

In some embodiments, the reference profile is based upon healthy samples taken during routine monitoring sampling. In some other embodiments, the reference profile may be a previously determined test profile. Suitably, the reference profile may be the test profile obtained from a first sample collected from a population of fish, which acts as a baseline for comparison with subsequent samples.

In some embodiments, the reference profile is the amount of the at least one analyte in a sample obtained from at least one fish of the population of fish not displaying any behavioural or physical characteristics of a condition or disease. In some embodiments the reference profile is the test profile of a first or subsequent sample form a population of fish which is used to establish a baseline.

### Analytes

Suitably, in some embodiments, the method according to the first, second, third, fourth or fifth aspect, the at least one analyte is any of those as defined in steps (a) or (b). In some embodiments, the method according to the first, second, third fourth, or fifth aspect comprises analysing a sample collected from said at least one fish from a population of fish to determine the amount of aspartate aminotransferase and optionally one or more of chloride, iron, sodium, total cholesterol, potassium, glucose, creatine kinase, lactate dehydrogenase, alanine aminotransferase, lactate, zinc, phosphate, albumin, creatine kinase-myocardial band, lipase, calcium, and magnesium. In some embodiments, the method according to the first, second, third fourth, or fifth aspect comprises analysing a sample collected from an individual fish to determine the amount of alanine aminotransferase and optionally one or more of chloride, iron, sodium, total cholesterol, potassium, glucose, creatine kinase, lactate dehydrogenase, aspartate aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of amount of iron and optionally one or more of chloride, aspartate aminotransferase, sodium, total cholesterol, potassium, glucose, creatine kinase, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of amount of glucose and optionally one or more of chloride, aspartate aminotransferase, sodium, total cholesterol, potassium, iron, creatine kinase, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of amount of creatine kinase and optionally one or more of chloride, aspartate aminotransferase, sodium, total cholesterol, potassium, iron, glucose, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from at least one fish from a said population of fish to determine the amount of amount chloride and optionally one or more of creatine kinase, aspartate aminotransferase, sodium, total cholesterol, potassium, iron, glucose, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of sodium and optionally one or more of creatine kinase, aspartate aminotransferase, chloride, total cholesterol, potassium, iron, glucose, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of total cholesterol and optionally one or more of creatine kinase, aspartate aminotransferase, chloride, sodium, potassium, iron, glucose, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of magnesium and optionally one or more of creatine kinase, aspartate aminotransferase, chloride, sodium, potassium, iron, glucose, lactate dehydrogenase, alanine aminotransferase, lactate, zinc, phosphate, albumin, creatine kinase-myocardial band, lipase, calcium, and total cholesterol.

In some embodiments, the method according to the first, second, third, fourth or fifth aspect comprises analysing a sample collected from said at least one fish from a population of fish to determine the amount of calcium and optionally one or more of creatine kinase, aspartate aminotransferase, chloride, sodium, potassium, iron, glucose, lactate dehydrogenase, alanine aminotransferase, lactate, zinc, phosphate, albumin, creatine kinase-myocardial band, lipase, magnesium, and total cholesterol.

In some embodiments, the method according to the first, second, third, fourth or fifth aspect comprises analysing a sample collected from said at least one fish from a population of fish to determine the amount of zinc and optionally one or more of creatine kinase, aspartate aminotransferase, chloride, sodium, potassium, iron, glucose, lactate dehydrogenase, alanine aminotransferase, lactate, calcium, phosphate, albumin, creatine kinase-myocardial band, lipase, magnesium, and total cholesterol.

In some embodiments, the method according to the first, second, third, fourth or fifth aspect comprises analysing a sample collected from said at least one fish from a population of fish to determine the amount of creatine kinase-myocardial band and optionally one or more of creatine kinase, aspartate aminotransferase, chloride, sodium, potassium, iron, glucose, lactate dehydrogenase, alanine aminotransferase, lactate, calcium, phosphate, albumin, zinc, creatine kinase-myocardial band, lipase, magnesium, and total cholesterol.

In some embodiments, the method according to the first, second, third, fourth or fifth aspect comprises analysing a sample collected from said at least one fish from a population of fish to determine the amount of potassium and optionally one or more of creatine kinase, aspartate aminotransferase, chloride, sodium, creatine kinase-myocardial band, iron, glucose, lactate dehydrogenase, alanine aminotransferase, lactate, calcium, phosphate, albumin, zinc, creatine kinase-myocardial band, lipase, magnesium, and total cholesterol.

In some embodiments, the method according to the first, second, third, fourth or fifth aspect comprises analysing a sample collected from said at least one fish from a population of fish to determine the amount of lactate and optionally one or more of creatine kinase, aspartate aminotransferase, chloride, sodium, creatine kinase-myocardial band, iron, potassium, lactate dehydrogenase, alanine aminotransferase, glucose, calcium, phosphate, albumin, zinc, creatine kinase-myocardial band, lipase, magnesium, and total cholesterol.

In some embodiments, the method according to the first, second, third, fourth or fifth aspect comprises analysing a sample collected from said at least one fish from a population of fish to determine the amount of lipase and optionally one or more of creatine kinase, aspartate aminotransferase, chloride, sodium, creatine kinase-myocardial band, iron, potassium, lactate dehydrogenase, alanine aminotransferase, glucose, calcium, phosphate, albumin, zinc, creatine kinase-myocardial band, lactate, magnesium, and total cholesterol.

In some embodiments, the method according to the first, second, third, fourth or fifth aspect comprises analysing a sample collected from said at least one fish from a population of fish to determine the amount of phosphate and optionally one or more of creatine kinase, aspartate aminotransferase, chloride, sodium, creatine kinase-myocardial band, iron, potassium, lactate dehydrogenase, alanine aminotransferase, glucose, calcium, lipase, albumin, zinc, creatine kinase-myocardial band, lactate, magnesium, and total cholesterol.

In some embodiments, the method according to the first, second, third, fourth or fifth aspect comprises analysing a sample collected from said at least one fish from a population of fish to determine the amount of albumin and optionally one or more of creatine kinase, aspartate aminotransferase, chloride, sodium, creatine kinase-myocardial band, iron, potassium, lactate dehydrogenase, alanine aminotransferase, glucose, calcium, lipase, phosphate, zinc, creatine kinase-myocardial band, lactate, magnesium, and total cholesterol.

In some embodiments, the method according to the first, second, third, fourth or fifth aspect comprises analysing a sample collected from said at least one fish from a population of fish to determine the amount of any two or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium.

In some embodiments, the method according to the first, second, third, fourth or fifth aspect comprises analysing a sample collected from said at least one fish from a population of fish to determine the amount of aspartate aminotransferase, iron, glucose, creatine kinase, chloride, zinc and total cholesterol.

In some embodiments, the method according to the first, second, third, fourth or fifth aspect comprises analysing a sample collected from said at least one fish from a population of fish to determine the amount of aspartate aminotransferase, iron, glucose, creatine kinase, chloride, sodium and total cholesterol.

In some embodiments, the method according to the first, second, third, fourth or fifth aspect comprises analysing a sample collected from said at least one fish from a population of fish to determine the amount of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium.

In some embodiments of any aspect of the invention described herein, the method comprises analysing a sample collected from said at least one fish from a population of fish to determine the amount of total cholesterol and any one or more of chloride; iron; sodium; aspartate aminotransferase; potassium; glucose; creatine kinase; lactate dehydrogenase; alanine aminotransferase; lactate; zinc; phosphate; albumin; creatine kinase-myocardial band; lipase; calcium; and magnesium.

In some embodiments of any aspect of the invention described herein, the method comprises analysing a sample collected from said at least one fish from a population of fish to determine the amount of glucose and any one or more of total cholesterol; chloride; iron; sodium; aspartate aminotransferase; potassium; creatine kinase; lactate dehydrogenase; alanine aminotransferase; lactate; zinc; phosphate; albumin; creatine kinase-myocardial band; lipase; calcium; and magnesium.

In some embodiments of any aspect of the invention described herein, the method comprises analysing a sample collected from said at least one fish from a population of fish to determine the amount of lipase and any one or more of total cholesterol; chloride; iron; sodium; aspartate aminotransferase; potassium; creatine kinase; lactate dehydrogenase; alanine aminotransferase; lactate; zinc; phosphate; albumin; creatine kinase-myocardial band; glucose; calcium; and magnesium.

In some embodiments of any aspect of the invention described herein, the amount of any given analyte is determined by clinical biochemistry techniques such as those described in the examples below or using techniques commonly known in the art.

### Computer Implemented Methods

### Training Methods

### Model0—An Artificial Intelligence Model for Determining Fish Health Status

In a sixth aspect, there is provided a computer implemented method of training a model to stratify a population of fish according to health status, wherein the method comprises:
(a) determining an amount of at least one analyte in a plurality of samples obtained from a population of fish to determine the health status of a population of fish;
(b) producing a training dataset of analytes associated with known healthy samples and unhealthy samples based on (a); and
(c) training the model using the training dataset from (b).

In some embodiments, the computer implemented method comprises an additional step wherein the health status of the population of fish is validated by farmer observation. In some embodiments, the health status of the population of fish is validated by conventional diagnostic techniques such as PCR or histology.

Suitably, in some embodiments there is provided a computer implemented method of training a model to stratify a population of fish according to health status, wherein the method comprises:
(a) determining an amount of at least one analyte in a plurality of samples obtained from a population of fish to determine the health status of a population of fish;
(b) validating the health status of the population of fish by farmer observation;
(c) producing a training dataset of analytes associated with known healthy samples and unhealthy samples based on (a) and (b); and
(d) training the model using the training dataset from (c).

In another embodiment, there is provided a computer implemented method of training a model to stratify a population of fish according to health status, wherein the method comprises:
(a) determining an amount of at least one analyte in a plurality of samples obtained from a population of fish to determine the health status of a population of fish; wherein a score of 0 is allocated to a healthy sample and a score of 1 is allocated to an unhealthy sample; and
(b) validating the health status of the population of fish by farmer observation; wherein a score of 0-0 is allocated to a sample deemed healthy in step (a) and healthy by the farmer and a score of 1-1 is allocated to a sample deemed unhealthy in step (a) and by the farmer;
(c) producing a training dataset wherein a score of 0-0 is indicative of a healthy sample and wherein a score of 1-1 is indicative of an unhealthy sample; and
(d) training the model using the training dataset from (c).

In some embodiments, the health status of the population of fish in step (a) may be determined by using the method of the first aspect as described herein. In some embodiments, the health status of a population of fish is determined by clinical biochemistry techniques such as those described in the examples below or using techniques commonly known in the art. Suitably, in some embodiments the one or more analytes are any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. Suitably, in some embodiments, any two or more, three or more, four or more, five or more, six or more, seven or more, eight or more, nine or more, ten or more, eleven or more, twelve or more, thirteen or more, fourteen or more, fifteen or more, sixteen or more, seventeen or more or eighteen analytes selected from the list comprising: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium are used to determine the health status of the population of fish.

In the above methods, the model may be developed by any appropriate method known by the skilled person. Such methods may include supervised learning methods, unsupervised learning methods or reinforcement learning methods. An example of a suitable method is described in Example 3 below. Suitably, in some embodiments the model is based on a single random forest classifier. In some embodiments, the model is trained using a smote technique (Chawla et al. 2002; SMOTE—Python v3.8.13, Version 0.11.0, n.d). In some embodiments, the model is trained using a smote and tomeklink sampling technique (Tomek, 1976; (TomekLinks—Python v3.8.13 Version 0.11.0, n.d.). In another embodiment, the model is trained using a smote and tomeklink sampling technique and a single random forest classifier.

In another embodiment, the model is trained using a smote technique and a GridSearch learner (Python v3.8.13, Sklearn.Model_Selection. GridSearchCV, n.d.). In one embodiment, the model is trained using smote and tomeklink sampling, a single random forest classifier and a GridSearch learner. In such embodiments, applying a GridSearch learner may improve the training accuracy of the model. Suitably, in some embodiments, applying a GridSearch learner improves the accuracy of the health status prediction to at least 80%, at least 85%, at least 90%, at least 95%, at least 96%, at least 97%, at least 98%, at least 99% or 100% accuracy.

In some embodiments, the model has advantageously been developed using data collected from fish samples that are known to be from healthy or unhealthy fish. In some embodiments, the method requires a two-step validation of fish health whereby only samples that both the fish farmer observations and the amount of at least one analyte (e.g., the test profile) are in agreement on health status are used to train the model. In some embodiments, a score of 0 represents healthy and a score 1 represents unhealthy. In a preferred embodiment, the model is trained on healthy scores of 0-0, i.e., fish farmer observations and amount of at least one analyte each allocating a score of 0 or unhealthy scores of 1-1, i.e., fish farmer observations and amount of at least one analyte each allocating a score of 1. In alternative embodiments, the skilled person can obtain different prediction models corresponding to different clinical situations. For example, a model can be developed using data collected only from fish at diagnosis of a particular health challenge using traditional methods such as histology or PCR, another model can be developed using data only from blood biochemistry analysis; another method may be developed using data only based on farmer observations.

### Model1

In a seventh aspect, there is provided herein a computer implemented method of training a model to predict a health challenge in a population of fish, wherein the method comprises:
(a) diagnosing at least one fish from a population of fish with one or more health challenges;
(b) determining an amount of at least one analyte obtained from the at least one fish from the population of fish;
(c) produce a training dataset based on (a) and (b);
(d) training the model based on training dataset from (c).

Suitably, in some embodiments of the method of training as described herein, the population of fish are a healthy population of fish. Alternatively, in a preferred embodiment, the population of fish are an unhealthy population of fish.

In some embodiments, a health challenge is a disease or a condition. In some embodiments, the diagnosis of a health challenge in the population of fish in step (a) may be determined by using the method of the fourth aspect as described herein. In some embodiments, the diagnosis of a health challenge in a population of fish is determined by clinical biochemistry techniques such as those described in the examples below or using techniques commonly known in the art. In a preferred embodiment, the diagnosis of a health challenge in a population of fish is determined by measuring the amount of one or more analytes in a sample obtained from at least one fish from a population of fish. Suitably, the one or more analytes are any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. The skilled person will understand that any of the embodiments of the methods of diagnosis as described above may be used to diagnose the at least one fish from the population of fish with one or more health challenges.

Suitably, in some embodiments the health challenge is any one or more of gill issues, pancreas disease, cardiomyopathy syndrome, heart and skeletal muscle inflammation and/or bacterial infection. Suitably, in some embodiments the health challenge is the terminal phase of a health challenge. In such embodiments, the terminal phase of a health challenge is classified as moribund.

In the above method, the model may be developed by any appropriate method known by the skilled person. Such methods may include supervised learning methods, unsupervised learning methods or reinforcement learning methods. An example of a suitable method is described in Example 3 below. Suitably, in some embodiments the model is based on an ensemble model (Python v3.8.13, Sklearn.Ensemble.StackingClassifier, n.d.). In some embodiments, the ensemble model is based on stacked Random Forest leaners. In some embodiments the model is based on sub-models. Suitably, in some embodiments there are at least one, at least two, at least three, at least four, at least five, at least six, at least seven, at least eight, at least nine or at least ten sub-models. In one embodiment, there are four sub-models. Suitably, in some embodiments the sub-models are based on random forest learners. In some embodiments, each sub-model is trained on disease training datasets. Suitably, in some embodiments, a disease training datasets comprises a dataset of unhealthy samples with diagnosed gill issues, cardiomyopathy, pancreas disease or heart and skeletal muscle inflammation. In an alternative embodiment, a sub-model may be trained on an unhealthy sample with diagnosed bacterial infection. In an another embodiment, a sub-model may be trained on an unhealthy sample with gill issues associated with an environmental challenge. Suitably, the environmental challenge may be a plankton bloom such as an algal bloom or a micro-jellyfish bloom. In some embodiments, the environmental challenge may be low water oxygen levels. In some embodiments, the environmental challenge may be a plankton bloom and low water oxygen levels. In some embodiments, the health challenge is diagnosed by clinical biochemistry such as biomarker analysis. In some embodiments, the health challenge is diagnosed by using the method of the fourth aspect of the present invention. In some embodiments, the health challenge is diagnosed by conventional diagnostic techniques known to the skilled person. Such techniques include but are not limited to histopathology and PCR assays.

In some embodiments, the sub-models are stacked to form a single model. Suitably, in such embodiments, the ensemble learner may be used to form a stacked single model. Suitably, in some embodiments the stacked single model is trained on unhealthy samples with a diagnosed health challenge. Suitably, in some embodiments the health challenge is any one or more of gill issues, gill issues associated with an environmental challenge, cardiomyopathy, pancreas disease or heart and skeletal muscle inflammation, or bacterial infection.

In some embodiments, a multi-label classifier may be used on the stacked single model. This may be advantageous as a fish from a population of fish may have more than one health challenge. Suitably, in some embodiments a tailored and improved PowerLabelSet derived from GridSearch may be used for multi-label classification. Suitably, in one embodiment, PowerLabelSet (Szymanski, 2017) (Python v3.8.13, Scikit-multilearn v0.20, n.d.) ( ) is used as the base classifier to make the health challenge prediction.

The skilled person will understand where stacking is used, the stacking model process is based on prediction from the sub-models, and then those predictions are implemented as features for a higher-level meta-model. In some embodiments the meta-model is logistic regression.

To the inventors' knowledge, training an AI model to detect and/or predict health status and identify health challenges such as HSMI, PD, CMS, bacterial infection and gill issues is previously unknown. The ability to train models on clinical chemistry data obtained from fish and accurately predict health status and identify specific health challenges was unexpected not least due to the former technical challenges in obtaining reliable and reproduceable clinical chemistry data from fish and the complex interplay of biomarkers across health challenges.

In some embodiments of any one of the sixth or seventh aspects as described herein, any one or more analytes in a sample may be measured using techniques as described herein or those commonly known in the art. Suitably, in some embodiments, the one or more analytes are any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. In some embodiments, the training dataset comprises the amount of any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium obtained from a healthy fish. Alternatively, or in addition to, in some embodiments, the training dataset comprises the amount of any one or more of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium obtained from an unhealthy fish. In some embodiments, the training dataset includes at least one, at least two, at least three, at least four, at least five, at least six, at least seven, at least eight, at least nine, at least ten, at least eleven, at least twelve, at least thirteen, at least fourteen, at least fifteen, at least sixteen at least seventeen or at least eighteen analytes selected from chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium and magnesium. In one embodiment, the method comprises measuring the amount of a plurality of analytes in a sample. In some embodiments, the training dataset comprises the amount of at least one analyte, at least two analytes, at least three analytes, at least four analytes, at least five analytes, at least six analytes, at least seven analytes, at least eight analytes, at least nine analytes, at least ten analytes, at least eleven analytes, at least twelve analytes, at least thirteen analytes, at least fourteen analytes, at least fifteen analytes, at least sixteen analytes, at least seventeen analytes or at least eighteen analytes in a sample. In one embodiment, the training dataset comprises the amount of chloride, glucose, iron, sodium, creatine kinase, aspartate aminotransferase and total cholesterol. Alternatively, in one embodiment, the training dataset comprises the amount of chloride, glucose, iron, zinc, creatine kinase, aspartate aminotransferase and total cholesterol. In another embodiment, the training dataset comprises the amount of chloride, iron, aspartate aminotransferase, sodium, creatine kinase, glucose, total cholesterol, lactate dehydrogenase and lactate. In another embodiment, the training dataset comprises the amount of chloride, iron, aspartate aminotransferase, sodium, creatine kinase, glucose, total cholesterol, lactate dehydrogenase, lactate, alanine aminotransferase, phosphate, albumin, creatine kinase-myocardial band, potassium and zinc. In yet another embodiment, the training dataset comprises the amount of chloride, iron, aspartate aminotransferase, sodium, creatine kinase, glucose, total cholesterol, lactate dehydrogenase, lactate, alanine aminotransferase, phosphate, albumin, creatine kinase-myocardial band, potassium, zinc, lipase, calcium and magnesium.

In some embodiments of any one of sixth or seventh aspects as described herein, the trained model may be a trained machine learning model. Suitably, in some embodiments, the trained machine learning model may be trained to provide a binary classification of a sample i.e., is the sample healthy or unhealthy. In another embodiment, the trained machine learning model may be trained to provide a probability value indicating a welfare score such as good health, weak health or critical health. In another embodiment, the trained machine learning model may be trained to provide a diagnosis of a health challenge in a sample. Suitably, in some embodiments, the trained machine learning model may be trained to first classify healthy and unhealthy samples and then stratify unhealthy samples into categories of gill issues, gill issues associated with an environmental challenge, heart and skeletal muscle inflammation, cardiomyopathy syndrome, pancreas disease and/or bacterial infection.

In a further aspect as described herein, there is provided trained model that has been obtained by the method of the sixth or seventh aspects as described herein.

### Model0—An Artificial Intelligence Model for Determining Fish Health Status

In an eighth aspect, there is provided herein a computer implemented method for determining the health status of at least one fish from a population of fish, wherein the method comprises:
(a) measuring or providing the amount of a plurality of analytes in at least one sample obtained from a population of fish;
(b) inputting the amount of the plurality of analytes from step (a) to an algorithm which correlates analyte amount with fish health status; and
(c) assigning a health status to the at least one fish from the population of fish.

In some embodiments, the plurality of analytes are selected from any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. In some embodiments, the plurality of analytes comprises at least two or more, at least three of more, at least four or more, at least five or more, at least six or more, at least seven or more, at least eight or more, at least nine or more, at least ten or more, at least eleven or more, at least twelve or more, at least thirteen or more, at least fourteen or more, at least fifteen or more, at least sixteen or more, at least seventeen or more or at least eighteen or more analytes selected from: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. In one embodiment, the computer implemented method comprises measuring or providing the amount of chloride, glucose, iron, sodium, creatine kinase, aspartate aminotransferase and total cholesterol in at least one sample obtained from a population of fish. Alternatively, in one embodiment, the computer implemented method comprises measuring or providing the amount of chloride, glucose, iron, zinc, creatine kinase, aspartate aminotransferase and total cholesterol. In another embodiment, the computer implemented method comprises measuring or providing the amount of chloride, iron, aspartate aminotransferase, sodium, creatine kinase, glucose, total cholesterol, lactate dehydrogenase and lactate. In another embodiment, the computer implemented method comprises measuring or providing the amount of chloride, iron, aspartate aminotransferase, sodium, creatine kinase, glucose, total cholesterol, lactate dehydrogenase, lactate, alanine aminotransferase, phosphate, albumin, creatine kinase-myocardial band, potassium and zinc. In yet another embodiment, the computer implemented method comprises measuring or providing the amount of chloride, iron, aspartate aminotransferase, sodium, creatine kinase, glucose, total cholesterol, lactate dehydrogenase, lactate, alanine aminotransferase, phosphate, albumin, creatine kinase-myocardial band, potassium, zinc, lipase, calcium and magnesium. The plurality of analytes suitably corresponds to the analytes in a test profile or reference profile as discussed above.

The skilled person will understand that the method may also be performed on an individual fish that may or may not be part of a population of fish.

The method may be performed using data collected from fish using methods such as histology or PCR or farmer observation. It will be understood that where the model as described herein is trained using a dataset obtained from PCR assays, histology or farmer observations that the algorithm in step (b) is provided with a score, a descriptor or a numerical value corresponding to the assay performed.

In some embodiments, the algorithm is a trained algorithm. Suitably, in some embodiments the trained algorithm is a trained machine learning model. In one embodiment, the trained algorithm is trained according to the sixth aspect as described above. In some embodiments, the model assigns the at least one fish from the population of fish the label of healthy or unhealthy. In some embodiments, the model assigns the numerical value of 0 or 1 to the at least one fish from the population of fish. Suitably, in some embodiments the value 0 is healthy and the value 1 is unhealthy.

In an exemplary embodiment, the algorithm may assign a heathy status to a sample when the amount of aspartate aminotransferase, creatine kinase, chloride and sodium are below an assigned threshold and iron, glucose and total cholesterol are above an assigned threshold. Alternatively, the algorithm may assign an unhealthy status to a sample when the amount of aspartate aminotransferase, creatine kinase, chloride and sodium are above an assigned threshold and iron, glucose and total cholesterol are below an assigned threshold. Suitable assigned thresholds may include but are not limited to those in Table 11.

In some embodiments of the eighth aspect, the algorithm assigns a health status according to a single prediction probability value. Suitably, in some embodiments the single prediction probability value is an interval. For example, an interval may be 0-1, 0.1-0.9 or 0.2-0.8. The skilled person will understand that a probability value closer to 1 is indicative of an unhealthy fish and a probability value closer to 0 is indicative of a healthy fish. Such probability intervals may be used to further classify the population of fish. Suitably, there is provided herein a further method for determining a welfare score and/or providing a welfare score ratio of the population of fish based on the output from model0. Suitably, this model may be referred to as model2.

Suitably, in a ninth aspect, there is provided a computer implemented method for determining a welfare score, wherein the method comprises:
(a) determining the health status of at least one fish from a population of fish according to the eighth aspect;
(b) inputting the health status from (a) into an algorithm to determine a welfare score.

Suitably, some embodiments the algorithm is built from the confidence intervals of model0. In one embodiment, the algorithm determines a welfare score using the probability values from model0. Suitably, in some embodiments the welfare score is calculated as follows:
welfare score(WF)=max, if fish is unhealthy and max>0.75
welfare score(WF)=min if fish is healthy and min<0.35
welfare score(WF)=max or min, if fish min>0.35 and max<0.75

Suitably, the welfare score may be described by a health level category. Suitably, a health level category may be good health, weak health or critical health. In some embodiments, the good health category is a fish with a probability value between 0 and 0.3 as determined by model0. In another embodiment, the weak health category is a fish with a probability value between 0.3 and 0.7 as determined by model0. In yet another embodiment, the critical health category is a fish with a probability value between 0.7 and 1 as determined by model0. The skilled person will understand that the welfare score may be described by the health level category. Suitably, in some embodiments the welfare score of a fish is good, weak or critical.

In some embodiments, the good health category defines the highest level of the healthiness of a fish. In some embodiments, the weak health category defines an acceptable level of the healthiness a fish. In some embodiments, the critical health category defines a sick fish.

In a tenth aspect, there is a provided a computer implemented method for determining a population welfare value, wherein the method comprises:
(a) determining a plurality of welfare scores of according to the ninth aspect;
(b) inputting the plurality of welfare scores from step (a) into an algorithm to determine a population welfare value for a population of fish.

Suitably, in some embodiments, the plurality of welfare scores are determined from at least two, at least three, at least four, at least five, at least six, at least seven, at least eight, at least nine, at least ten, at least twenty, at least thirty, at least forty, at least fifty, at least sixty, at least seventy, at least eighty, at least ninety, at least one hundred, at least five hundred, at least one thousand, at least five thousand or at least ten thousand fish from a population of fish.

In some embodiments, the algorithm calculates the ratio of samples belonging to each health category to provide a population welfare value. Suitably, in some embodiments, the population welfare value is determined by the algorithm calculating the ratio of fish from a population of fish classified as good, weak or critical health. The skilled person will understand that the greater the number of samples categorised in the good health category will provide a healthy population welfare value. In contrast, where most samples belong to the weak health category then a weak health population welfare value is awarded to the population of fish. Where the majority of samples belong to the critical health category, then a critical health population welfare value is awarded to the population of fish. In one embodiments the population welfare value may be cumulative welfare scores of individual fish from a population of fish. In some embodiments the population welfare value comprises an arithmetical manipulation of the number of fish in each of the plurality of health level categories, e.g. the number of healthy fish divided by the number of unhealthy fish.

In some embodiments a good or healthy population welfare value is indicated by a high proportion or high ratio of fish in the healthy or good health category. In some embodiments, an intermediate or weak health population welfare value is indicated by a high proportion or high ratio of fish in the weak health category. In some embodiments, a poor health population welfare value is indicated by a high ratio or proportion of fish in the unhealthy or critical health category.

### Model1—An Artificial Intelligence Model for Predicting Fish Health Challenges

In an eleventh aspect, there is provided herein a computer implemented method for predicting a health challenge in at least one fish of a population of fish; wherein the method comprises:
(a) measuring or providing the amount of a plurality of analytes from at least one sample obtained from at least one fish of a population of fish;
(b) inputting the amount of the plurality of analytes from step (a) to an algorithm which correlates analyte amount with fish health challenges; and
(c) assigning a health challenge to the at least one fish of a population of fish.

In some embodiments, the plurality of analytes selected from any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium. In some embodiments, the plurality of analytes comprises at least two or more, at least three of more, at least four or more, at least five or more, at least six or more, at least seven or more, at least eight or more, at least nine or more, at least ten or more, at least eleven or more, at least twelve or more, at least thirteen or more, at least fourteen or more, at least fifteen or more, at least sixteen or more, at least seventeen or more or at least eighteen or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium.

In one embodiment, the computer implemented method comprises measuring or providing the amount of glucose, aspartate aminotransferase, potassium, alanine aminotransferase, lactate, albumin and iron for inputting into the algorithm in step (b).

In one embodiment, the computer implemented method comprises measuring or providing the amount of glucose, aspartate aminotransferase, potassium, alanine aminotransferase, lactate, zinc and creatine kinase-myocardial band for inputting into the algorithm in step (b).

In one embodiment, the computer implemented method comprises measuring or providing the amount of aspartate aminotransferase, phosphate, creatine kinase, alanine aminotransferase, zinc, chloride and creatine kinase-myocardial band for inputting into the algorithm in step (b).

In one embodiment, the computer implemented method comprises measuring or providing the amount of glucose, phosphate, magnesium, lactate, total cholesterol, iron and calcium for inputting into the algorithm in step (b).

In one embodiment, the computer implemented method comprises measuring or providing the amount of glucose, aspartate aminotransferase, potassium, alanine aminotransferase, lactate, albumin, iron, zinc, creatine kinase-myocardial band, phosphate, chloride, magnesium, total cholesterol and calcium for inputting into the algorithm in step (b).

In some embodiments, the algorithm is a trained algorithm. Suitably, in some embodiments the trained algorithm is a trained machine learning model. In one embodiment, the trained algorithm is trained according the sixth aspect as described above. In some embodiments, the algorithm assigns the at least one fish from the population of fish the label of gill issues, cardiomyopathy syndrome, pancreas disease, heart and skeletal muscle inflammation. In an alternative embodiment, the algorithm assigns the at least one fish from the population of fish the label of gill issues, cardiomyopathy syndrome, pancreas disease, heart and skeletal muscle inflammation or bacterial infection. In an another embodiment, the algorithm assigns the at least one fish from the population of fish the label of gill issues, gill issues associated with an environmental challenge, cardiomyopathy syndrome, pancreas disease, heart and skeletal muscle inflammation or bacterial infection.

In an exemplary embodiment, in step (c) of the method, the algorithm assigns the health challenge as gill issues when glucose, alanine aminotransferase and iron are below an assigned threshold and aspartate aminotransferase, potassium, lactate, albumin and alanine aminotransferase are above an assigned threshold as determined in step (b). Suitable assigned thresholds may include, but are not limited to, those in Table 13.

In an exemplary embodiment, in step (c) of the method, the algorithm assigns the health challenge as gill issues associated with plankton bloom when glucose, alanine aminotransferase, calcium and iron are below an assigned threshold and aspartate aminotransferase, potassium, lactate, albumin and alanine aminotransferase are above an assigned threshold as determined in step (b). Suitable assigned thresholds may include, but are not limited to, those in Table 13.

In another exemplary embodiment, in step (c) of the method, the algorithm assigns the health challenge as cardiomyopathy syndrome when aspartate aminotransferase, lactate, potassium, creatine kinase-myocardial band, zinc and alanine aminotransferase are above an assigned threshold and glucose is below an assigned threshold as determined in step (b).

Suitable assigned thresholds may include, but are not limited to, those in Table 13.

In yet another exemplary embodiment, in step (c) of the method, the algorithm assigns the health challenge as pancreas disease when aspartate aminotransferase, phosphate, creatine kinase-myocardial band, creatine kinase and chloride are above an assigned threshold and zinc and alanine aminotransferase are below an assigned threshold as determined in step (b). Suitable assigned thresholds may include, but are not limited to, those in Table 13.

In another exemplary embodiment, in step (c) of the method, the algorithm assigns the health challenge as heart and skeletal muscle inflammation when phosphate, magnesium and lactate are above an assigned threshold and glucose, total cholesterol, iron and calcium are below an assigned threshold as determined in step (b). Suitable assigned thresholds may include, but are not limited to, those in Table 13.

In some embodiments of the computer implemented method as described herein, the health status of the at least one sample is known to be obtained from a healthy fish or an unhealthy fish. Suitably, in one embodiment the at least one sample is obtained from an unhealthy fish.

Suitably, in one embodiment the at least one sample is obtained from healthy fish. In another embodiment, the health status of the at least one sample is unknown prior to predicting a health challenge. In one embodiment, at least one sample is obtained from an individual fish that may or may not be part of a population of fish.

The method may be performed using data collected only from fish using methods such as histology or PCR or farmer observation. It will be understood that where the model as described herein is trained using a dataset obtained from PCR assays, histology or farmer observations that the algorithm in step (b) is provided with a score, a descriptor or a numerical value corresponding to the assay performed.

### Computer Programs

In a further aspect, there is provided a computer program comprising instructions which, when the program is executed by a computer, cause the computer to carry out the method of determining the health status of at least one fish from a population of fish according to the eighth aspect.

In another aspect, there is provided herein a computer program comprising instructions which, when the program is executed by a computer, cause the computer to carry out the method of determining a welfare score according to the ninth aspect.

In another aspect, there is provided herein a computer program comprising instructions which, when the program is executed by a computer, cause the computer to carry out the method of determining a welfare score ratio according to the tenth aspect.

In yet another aspect, there is provided herein a computer program comprising instructions which, when the program is executed by a computer, cause the computer to carry out the method of predicting a health challenge in at least one fish from a population of fish according the eleventh aspect.

It will be understood by the person skilled in the art that the processing (e.g., determining health status, predicting a health challenge and/or determining a welfare score) of the sample data may be performed by processing circuitry of the computer. The processing circuitry of the computer may be communicatively coupled to a memory. The memory may store the trained model, preferably in some embodiments a trained machine learning model. The processing circuitry may process the sample data using the trained machine learning model stored in the memory. The processing circuitry may comprise general purpose processor circuitry configured by program code to perform specified processing functions. Alternatively, the processing circuitry may comprise special purpose processing circuitry. Thus, the configuration of the circuitry to perform its specified function may be limited exclusively to hardware, limited exclusively to software, or a combination of hardware modification and software execution. Program instructions may be used to configure the logic gates of general purpose or special purpose processor circuitry to perform the processing functions. The software, where provided, may be provided in the form of a computer program product comprising instructions which, when the program is executed by the processing circuitry, cause the processing circuitry to carry out its specified function. The software, where provided, may be stored on one or more (e.g., non-transitory) computer-readable storage media.

Suitably, in some embodiments, there is provided herein one or more non-transitory computer-readable storage media comprising instructions, which, when executed by one or more processors, cause the processors to carry out the method of the eighth aspect.

Suitably, in some embodiments, there is provided herein one or more non-transitory computer-readable storage media comprising instructions, which, when executed by one or more processors, cause the processors to carry out the method of the ninth aspect.

Suitably, in some embodiments, there is provided herein one or more non-transitory computer-readable storage media comprising instructions, which, when executed by one or more processors, cause the processors to carry out the method of the tenth aspect.

Suitably, in some embodiments, there is provided herein one or more non-transitory computer-readable storage media comprising instructions, which, when executed by one or more processors, cause the processors to carry out the method of the eleventh aspect.

In another aspect there is provided herein a trained model obtained by the method of the sixth or seventh aspects.

### Therapeutic Interventions

The various methods described above may be followed by treating the population of fish to treat, prevent or mitigate an undesirable health status, condition or diseases.

Accordingly, in a further aspect of the present invention, there is provided a method of treating a fish or a population of fish comprising administering an effective amount of a therapeutic agent to the population of fish to treat the change in health status or the condition or disease as determined according to any of the various aspects or embodiments as discussed above.

The term ‘treatment’ or ‘treating’ as used herein may refer to reducing, ameliorating or eliminating one or more signs, symptoms, or effects of a disease or condition. ‘Treatment’ as used herein includes any treatment of a disease in a fish or a population of fish, and includes: (a) preventing the disease from occurring in a fish or a population of fish predisposed to the disease or at risk of acquiring the disease but has not yet been diagnosed as having it; (b) inhibiting the disease, i.e., arresting its development; (c) relieving the disease, i.e., causing regression of the disease; and (d) alleviating or reducing any symptoms of the disease.

In a further aspect, there is provided a method of treating a condition or disease in a population of fish identified as having a change in health status or as having a condition or disease by any of the aspects or embodiments described herein.

In some embodiments, the disease or condition is pancreas disease, cardiomyopathy syndrome, gill issues (including but not restricted to the specific amoebic gill disease, parasitic gill disease, viral gill disease, bacterial gill disease, zooplankton (e.g., cnidarian nematocyst)-associated gill disease, harmful algal gill disease and chemical/toxin-associated gill disease and the less specific complex gill disease), heart and skeletal muscle inflammation and/or bacterial infection.

Fish welfare has become increasingly important in aquaculture and continuous health monitoring during sea lice treatments is essential to ensure fish wellbeing. Clinical chemistry analysis has been previously undertaken in salmonids (Hille, S. A, 1982) and has proven a useful tool to analyse the health status of fish following pollution exposure (Bernet, D et al., 2000), feed trials (Ferri, J. et al 2011; Adel, M. et al, 2015), disease monitoring and diagnosis (Rehulka, J. 2003; Floyd-Rump, T. P. et al., 2017), toxicological studies (Steinbach, C. et al. 2014; Javed, M. et al., 2017) and to investigate pathophysiology (Benfey, T. J. & Biron, M., 2000).

It is preferable in some circumstances to not treat or to delay treatment of a population of fish classified as ‘unhealthy’, ‘weak health’, ‘critical health’ or having been diagnosed with a condition or disease, with routine husbandry treatments including anti-parasite treatments such as anti-sea lice treatments, antibiotics or hydrogen peroxide. Treating fish which are already unhealthy with, for example, routine parasite treatments, may lead to undesirable mortality rates.

Anti-parasite treatments include but are not limited to levamisole, metronidazole or praziquantel. In some circumstances routine treatments administered to unhealthy fish populations increases the mortality rate.

It is preferable in some circumstances to only treat healthy fish or fish with a ‘good health’ welfare score with routine husbandry treatments to reduce mortality.

Accordingly, the present invention has the advantage of determining the health status of a population of fish, identifying or diagnosing a health challenge and/or the welfare score of a population of fish before treating with routine husbandry treatments. This may have the advantage of reducing the mortality rate of farmed fish. This may also have the advantage of reducing the amount unnecessary antibiotics, anti-parasitic drugs and chemical agents entering the aquatic ecosystem.

Suitably, according to one aspect of the present invention there is provided a method for determining whether or not to treat a population of fish, or determining whether a proposed treatment is appropriate, comprising:
(a) determining the health status of a population of fish according to the method of the first aspect or the eighth aspect;
(b) wherein when the health status of the population of fish is determined to be unhealthy in step (a) treatment is undesirable or an alternative treatment is recommended or, wherein when the fish is determined to be healthy in step (a), the proposed treatment is desirable.

The method may further comprise the step of treating said population of fish when the health status of the population is determined to be healthy. Thus, the method may be a method of treating a population of fish, e.g. to prevent or treat parasitic bacterial, amoebic and/or viral infection.

According to another aspect of the present invention there is provided a method for determining whether or not to treat a population of fish, or determining whether a proposed treatment is appropriate, comprising the steps of:
(a) determining the welfare score of a population of fish according to the method of the second, ninth or tenth aspect;
(b) wherein when the health status of the population of fish is weak or critical in step (a) treatment is undesirable or an alternative treatment is recommended, or wherein when the fish is determined to be healthy in step (a) the proposed treatment may proceed.

The method may further comprise the step of treating said population of fish when the health status of the population is determined to be healthy in step (a). Thus, the method may be a method of treating a population of fish, e.g. to prevent or treat parasitic bacterial, amoebic and/or viral infection.

In some embodiments, the health status or welfare score may indicate a suitable therapeutic window for a proposed treatment.

In some embodiments, a proposed treatment may include any routine animal husbandry treatments such as treatment for parasitic, bacterial, amoebic and/or viral infections.

In some embodiments, the population of fish should not be treated for a parasite infection. In a preferred embodiment, the population of fish should not be treated for a sea lice infection, or an alternative treatment method recommended. Parasites include but are not limited to ectoparasites such as sea lice or salmon fluke (Gyrodactylus salaris), endoparasites such as Kudoa thyrsites. Suitably, in some embodiments, the population of fish should not be treated with anti-parasitic treatments, preferably the population of fish should not be treated with anti-sea lice treatments. In some embodiments, the population of fish should not be treated with any one of levamisole, metronidazole or praziquantel.

In further embodiments, the population of fish should not be treated for a bacterial, amoebic and/or viral infection.

Suitably, in some embodiments, an alternative treatment method is providing a therapeutic agent to treat any of pancreas disease, cardiomyopathy syndrome, heart and skeletal muscle inflammation, gill issues and/or bacterial infection. Suitably, in some embodiments, the population of fish is first treated for any of pancreas disease, cardiomyopathy syndrome, heart and skeletal muscle inflammation, gill disease and/or bacterial infection, then the population of fish is treated with routine animal husbandry treatments.

Suitably, in some embodiments, it is determined that the population of fish should not be treated, and the population of fish should be harvested.

In any aspects and embodiments discussed herein, the health status, welfare score and/or diagnosis of a condition or disease may indicate that a population or a sub-population of fish should be harvested. In some aspects and embodiments, the health status, welfare score and/or diagnosis of a condition or disease may indicate that a population of fish require reduced feeding, a convalescence diet or early harvesting.

Harvesting as used herein may refer to slaughtering the population of fish or a sub-population of fish or individual fish. The sub-population of fish selected for harvesting may be the weakest or smallest fish. In some embodiments, the population of fish or a sub-population of fish or individual fish selected for harvesting may be moribund fish. Harvesting includes any of stunning, killing and further processing of fish. Further processing of fish may include preparing the fish for human or animal consumption.

Early harvesting refers to slaughtering of fish earlier than the full two-year production cycle. Suitably, early harvesting refers to slaughtering of fish before the fish have reached their full size and weight.

### Samples

The term ‘sample’ as used herein may refer to any biofluid. In some embodiments of any aspect of the present invention the sample is blood or a blood fraction, such as serum or plasma. Suitably the blood or blood fraction sample is from circulating blood. In some embodiments the sample is haemolymph. A ‘later sample’ as used herein may refer to any sample collected from any fish from the population of fish after the first sample. A later sample may include a first, second, third, fourth, fifth, sixth, seventh, eighth, ninth or tenth sample, collected after the first sample.

According to any one of the aspects described herein, a sample may be obtained from at least one fish from a population of fish. The skilled person would understand that ‘a sample’ as referred to herein may mean a sample obtained from one individual fish and/or samples obtained from more than one individual fish. Suitably, according to any suitable aspect of the invention as described herein, analysing a sample may include analysing a plurality of samples from a plurality of fish from a population of fish. Suitably, a plurality of fish may include at least one, least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand fish. Suitably, a plurality of samples may include at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, at least one hundred, at least two hundred, at least three hundred, at least four hundred, at least five hundred, at least one thousand, at least two thousand, at least five thousand samples obtained from one or more fish from a population of fish.

In some embodiments, samples are collected from at least one enclosure (e.g., cage, pen or tank) per site. Preferably, samples are collected from at least two, at least three, at least four, at least five, at least six, at least seven, at least eight, at least nine or at least ten enclosures per site.

Suitably the population of fish are wild, captive or farmed fish. Captive fish include domesticated fish kept in lakes, ponds, tanks and aquariums. In some embodiments, the fish are from the salmonid, cichlidae, carp or acipenseridae families. In some embodiments the population of fish are shellfish.

In some embodiments the population of fish are any one of salmon, brown trout, rainbow trout, tilapia, carp, minnows, sea bass, sea bream and/or sea bream.

In some embodiments the population of fish are any one of shrimp, oysters, lobsters, crabs, mussels or clams.

The skilled person will understand that it may be desirable perform certain aspects of the invention on an individual fish that is not part of a population of fish. Such fish may be an individual fish kept for breeding purposes, a pet fish or a fish of high value such as Koi Carp. Suitably, there is provided herein an alternative aspect of the first, third, fourth, eighth, ninth or eleventh aspects where any of the methods for determining the health status, monitoring health status, diagnosis of a condition or disease and/or providing a welfare score are performed on a single, individual fish that is not part of a population of fish.

### Kit

In one aspect, there is provided a kit for use in any of the aspects and embodiments of the present invention, wherein the kit comprises one or more reagents for determining the amount of the at least one analyte in a sample, and instructions for use.

In some embodiments, the kit comprises instructions for sample collection and processing. Suitably, in one embodiment the instructions for sample collection and processing comprise the steps of:
(a) Fish anaesthetised using MS-222 following manufacturer's instructions;
(b) Withdraw whole blood from a caudal vein, behind a caudal fin using a Sarstedt system;
(c) Insert the needle behind the caudal fin, towards fish spine. Stop when feel resistance, gently pull the plunger until it reaches the end what you can hear as “click” sound. That is the moment when the vacuum is created and at that point you need to find the vein by gently moving the needle to hit the site between the vertebrate under the spine (once you create the vacuum, do not take the needle out).
(d) Once you hit the spot leave the syringe and allow vacuum to withdraw the blood.
(e) After blood collection, remove needle from the syringe, place cap on needle and dispose of needle in sharp bin provided to you.
(f) Snap the clip of a syringe plunger invert tube ×3 (do not shake) to mix clotting activator and leave standing upright for a minimum of 30 min before centrifugation (Max clotting time 3 h).
(g) After 30 min, centrifuge for 10 minutes (at 3000-4000 g speed).
(h) Allow the centrifuge to come to complete stop. Remove tubes carefully without disturbing contents.
(i) Open the cap of a syringe and pipette serum into labelled Eppendorf tube (ensuring not to disturb the blood clot) make sure that you do not transfer red blood cells from the bottom of tube.
(j) 0.3 ml of serum is minimum required for biochemical analysis.
(k) Freeze the samples the same day after collection (−20 °C) and post as soon as possible.
(l) On the day of the postage: Place frozen samples in a bag together with flat freezer pack and wrap the bag in aluminium foil. Pack this into a thermal postal pocket.
(m) Fill in details on Sample Submission Form and include with samples.

In a further embodiment, the kit comprises thermal postal pockets and a freezer pack for transportation of the samples.

### BRIEF DESCRIPTION OF THE FIGURES

FIG. 1: An illustrative example of obtaining a blood sample from the caudal vein of a fish.

FIG. 2: An illustrative example of haemolysis levels in blood serum following centrifugation.

FIG. 3: Schematic diagram showing AI model-based system conception. The diagram illustrates model 0 where healthy and unhealthy samples are discriminated. Unhealthy samples are passed to model 1 where sub-models are constructed for the health challenges: pancreas disease, gill issues, cardiomyopathy and heart and skeletal muscle inflammation which are stacked into a single model for multi-label classification.

FIG. 4: SHAP summary plot showing the feature importance of biomarkers of model for a prediction case.

FIG. 5: Exemplary SHAP force plot filtered on CK-MB showing the value of biomarker affecting the fish heath prediction.

FIG. 6: Schematic diagram of AI model to determine fish welfare score. The diagram illustrates that samples are obtained and passed through model0 for binary classification into healthy or unhealthy and a prediction probability for each sample. The sample is then passed through model2 where the sample is classified into predefined health level ranges and single prediction probability. The welfare ratio is then calculated to provide a welfare score.

FIG. 7: Plot showing the distribution of probabilities of single prediction from a model0 for test dataset(s)

FIG. 8: Plot showing the probability distribution obtained from the mode0 on a test dataset X.

FIG. 9: Comparison of biomarker response as determined by clinical biochemistry analysis between healthy and unhealthy fish for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 10: Comparison of biomarker response as determined by clinical biochemistry analysis between healthy samples and samples obtained from fish with diagnosed pancreas disease for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 11: Comparison of biomarker response as determined by clinical biochemistry analysis between healthy samples and samples obtained from fish with diagnosed gill issues for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 12: Comparison of biomarker response as determined by clinical biochemistry analysis between healthy samples and samples obtained from fish exposed to plankton blooms for each of albumin; alanine aminotransferase; aspartate aminotransferase; calcium; total cholesterol; creatine kinase; creatine kinase-myocardial band; glucose; iron; lactate; lactate dehydrogenase; lipase; magnesium; phosphate; zinc; sodium; potassium; and chloride.

FIG. 13: Comparison of biomarker response as determined by clinical biochemistry analysis between healthy samples and samples obtained from fish with diagnosed heart and skeletal muscle inflammation for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 14: Comparison of biomarker response as determined by clinical biochemistry analysis between healthy samples and samples obtained from fish with diagnosed cardiomyopathy syndrome for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 15: Comparison of biomarker response as determined by clinical biochemistry analysis between healthy samples and samples obtained from fish with a bacterial infection for each of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

FIG. 16: Prediction probability plot of prediction value against standard deviation.

FIG. 17: Schematic diagram of the software system of model2.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

### DEFINITIONS

As referred to herein, the ‘difference’ in the test profile or the ‘difference’ in the at least one analyte as compared to the reference profile may refer to any positive or negative deviation from the reference value. The difference may refer to an increase in the amount of at least one analyte when compared to the reference profile. The difference may refer to a decrease in the amount of at least one analyte when compared to the reference profile. It will be clear to the skilled person that where more than one analyte is measured, the difference may refer to an increase of at least one analyte compared to the reference profile or a decrease of one or more different analytes when compared to the reference value. A difference may also be determined by deviation from the mean reference value. Suitably, a difference may refer to mean of the reference value ±1SD, 2SD, 3SD or ±4SD.

‘Healthy’ as described herein refers to a fish or a population of fish that is substantially absent of disease. Healthy fish may not display physical characteristics of a disease and may also have normal blood biochemistry. Healthy fish may also include fish that do not display physical characteristics of a disease but may present with one or more biomarkers as described herein falling out of the healthy range into the “warning” or “abnormal” range, i.e., not completely healthy or normal but not unhealthy.

‘Unhealthy’ as described herein refers to a fish or a population of fish wherein at least one biomarker as described herein lies in the unhealthy reference range. Unhealthy fish may or may not display physical symptoms of disease. Unhealthy fish may have a health challenge.

A health challenge may be defined as a condition or a disease. A health challenge as described herein includes, for example, pancreas disease, cardiomyopathy syndrome, gill issues, heart and skeletal muscle inflammation and bacterial infection. Gill issues may be caused by environmental ‘stresses’ or ‘challenges’ such as plankton blooms and/or low oxygen levels, parasites, bacterial, viruses or fungal diseases. Plankton blooms occur when one or more plankton species reproduces at a rapid rate, multiplying quickly in a short amount of time. This may reduce the oxygen concentration of the water. Plankton blooms are caused by species of phytoplankton such as microalgae and blue-green bacteria. Exemplary species of phytoplankton known to result in plankton blooms, include but are not limited to, Chaetoceros socialis, Ditylum brightwellii, Chaetoceros debilis, Chaetoceros affine, Skeletonema, Pseudo-Nitzschia Seriata, Gyrosigma, Coscinodiscus radiatus, Chaetoceros didymus, Ceratium Spp, Eucampia zodiacus, Detonula Pumila, chaetoceros curvisetus. Plankton blooms may also be caused by zooplankton such as species of micro-jellyfish. Exemplary species of zooplankton known to result in plankton blooms include single-celled dinoflagellate species such as Ceratium and hydrozoan jellyfish such as Lizzia blondina.

Pancreas disease, cardiomyopathy syndrome, heart and skeletal muscle inflammation are typically a result of viral infection but may be caused by other infectious agents. Bacterial infection includes any disease associated with bacterial infection of fish. Such infections include but are not limited to infections by Aeromonas salmonicida, Vibrio species such as Vibrio anguillarum and Vibrio salmonicida, Pasteurella skyensis Tenacibaculum maritimum, Flavobacterium psychrophilum, Piscirickettsia salmonis and Yersinia ruckeri. The terminal stage of any one of these challenges in an unhealthy fish is classified as moribund. A moribund fish may include a fish that presents with symptoms such as lethargy, laboured breathing, loss of buoyancy and erratic swimming. In some embodiments of the present invention, moribund fish presents with increased amounts of lactate, magnesium, phosphate, sodium, potassium and chloride and decreased amounts of albumin, total cholesterol, iron and zinc.

The term ‘therapeutic window’ as referred to herein may refer to the optimum time to treat the population of fish with a suitable therapy. The optimum time may refer to the timeframe when the treatment is most effective and when the risk of mortality is low.

The term ‘creatine kinase-myocardial band’ may be used interchangeably with ‘creatine kinase-MB’ or ‘CK-MB’. Creatine kinase-MB refers to an isoform of creatine kinase that is predominantly, but not exclusively, expressed in heart muscles.

The term ‘welfare score’ or ‘population welfare value’ as used herein may indicate the overall welfare of an individual fish or, more typically, a fish population. The welfare score may be based on the cumulative health scores or proportion of health scores of individual fish in a population. Welfare scores may be good health, weak health or critical health. The population welfare value, for example, be determined by calculating the ratio of fish from a population of fish classified as good, weak or critical health to provide a population wide welfare value.

The skilled person will understand ‘supervised learning’ as used herein to relate to training methods whereby a computer algorithm is trained on input data that has been labelled for a particular output. The model is trained until it can detect the underlying patterns and relationships between the input data and the output labels, enabling it to yield accurate labelling results when presented with unseen data. In contrast, the term ‘unsupervised learning’ relates to training methods whereby a computer algorithm presented with unlabelled data and is designed to detect patterns or similarities on its own. The term ‘reinforcement learning’ relates to methods of training models to make a sequence of decisions. In reinforcement learning, algorithm employs trial and error to come up with a solution to the problem. To get the machine to do what the programmer wants, the model gets either rewards or penalties for the actions it performs.

### INTRODUCTION

Clinical biochemistry is the cornerstone of human and veterinary medicine, used to measure the health status of organisms. Clinical biochemistry is the analysis of amounts (typically concentrations) of numerous proteins, metabolites, enzymes and electrolytes in bodily fluids, most commonly blood or blood-derived serum or plasma, for non-destructive diagnosis and monitoring of disease. Clinical biochemistry is a vital diagnostic tool, but despite occasional studies showing its usefulness in monitoring health status in Atlantic salmon (Salmo salar L.), it has not yet been widely utilized within the aquaculture industry based on (i) lack of established background (normal) levels and (ii) lack of clinically significance data. The inventors have generated a significant dataset to overcome both of these issues.

Biochemistry endpoints previously measured in salmonid species (Sandnes & Waagbo, 1988; Rehulka, 2003; Quinn et al., 2015; Braceland et al., 2017; Barisic et al., 2019; Marco Rozas-Serri, 2022). But there is no information available on the clinical significance of this approach to assess fish health in aquaculture. The present invention has taken existing human medical medium/high throughput clinical chemistry analysers with the software open to make the necessary changes to the settings to make the normal, medical kits used for these instruments fall within the reactive ranges for fish blood, established through research.

With a significant amount of data (18 clinical chemistry endpoints on ~10,000 fish serum samples) the present inventors have also developed three different AI models to aid with data interpretation, sample categorisation and health predictions. Firstly, a model was developed to categorise fish into healthy and unhealthy (model0). A further model was developed to further categorise and predict the most the specific health challenge based on the biomarker expression in unhealthy fish (model1). Data are also clinically interpreted through the comparison of biomarker expression between fish suffering from different health challenges.

The present invention also provides a novel welfare classification system (model2) that uniquely predicts the welfare score of a fish farm site by categorising inputs of from model0 to predict a population wide welfare score. This is particularly beneficial for fish farm manager who can then reliably understand the health of their stock at a population level and make data driven husbandry decisions.

The current methods for routine fish health assessment and disease identification in aquaculture rely on the use of lethal techniques such as tissue specific PCR and histopathology. The present invention provides methods for assessing and monitoring the health status of a population of fish and diagnosing a population of fish as having a condition or disease. The present invention has the advantage of using non-lethal, blood-based methods that are rapidly assessed using automated, medium/high throughput clinical chemistry instrumentation.

Results are interpreted against an extensive background database to enable clinical interpretation and the establishment of normal background ranges (typically in the form of a reference profile for the relevant markers/analytes). Results are conveniently presented via a traffic light system with green indicating within the normal ranges, yellow/amber indicating between 1-2 standard deviations (SD) outside of the normal range and red begin >2 SD from the mean. The AI models aid with data interpretation but can also valuably predict at a population level the welfare of the fish stock, which is not possible with lethal techniques currently available to fish farm managers.

Using this non-lethal based approach health monitoring of a population of fish from several pens at a site can be undertaken routinely in larger sample numbers providing an overview of the health of the population. The present invention provides a practical method for continuous fish health assessment, that is rapid, non-lethal, and a blood-based method to assess fish health, similar to human and veterinary medicine to augment and ultimately replace the existing slow, lethal histology methods.

Such an approach can be applied to salmon and other commercially important fish (e.g., sea bass, sea bream, sturgeon) and invertebrate (e.g., shrimp, lobster) aquaculture markets.

The problem faced by the aquaculture industry for the assessment of fish health is the dependence on slow (5-10 days), lethal, histology-based methods. The present invention provides a re-purposed human high throughput medical technology for use on fish blood to enable rapid clinical chemistry centred health assessment, based on the continuous sampling of fish stock, similar to that used in all other livestock-based agriculture. The skilled person will understand that it is not an insignificant challenge to apply continuous sampling to fish stocks, due to a lack of reference data for clinical comparison and blood sample collection difficulties.

These challenges have been largely overcome by the present invention.

The advantages of the present invention in some embodiments include:
Rapid results within 24 hours.
Enabling fish health managers to make data-informed husbandry decisions, facilitating predictive health forecasting, reducing mortality and increasing productivity.
Enabling the development of a pro-active fish healthcare model, augmenting the understanding of many health issues and ultimately replacing the need for histological-based analysis.

On-going and regular analysis of blood biomarkers can support existing work designed to continually improve fish welfare in commercial farming. By creating a standardised dataset and algorithm-based AI model to generate “early-warning” health indicators via a site-specific online platform within 24 hours of sample delivery, vets and fish health managers will be able to detect fish health issues earlier, thus increasing the likelihood of effective intervention by treatment, reduced feeding, convalescence diet or early harvest.

As this technique is non-lethal and automated, larger, more representative sample numbers can be analysed. Other advantages include:
The ability to assess the health of the whole fish (homeostasis), as well as the functioning of specific tissues (e.g., liver, kidney).
The cost-effective analysis of large sample numbers reduces the chances of a critical organ pathology being overlooked.
Data presentation via a traffic light system (green=healthy; orange=potential health issue; red=serious health issue requiring immediate attention).
Enables fish health managers to make data informed husbandry decisions, improving fish health, productivity and profitability.

This technique is enabled by altering the high throughput clinical chemistry instruments and biomarker reactive ranges to make them fish specific. The clinical chemistry biomarker results can then be interpreted through a substantial chemistry database for 33 biomarkers measured in thousands of salmon and trout samples, (including a 12-month sampling plan from ‘control’ sites to establish background levels). Additionally, machine learning models identify fish as healthy or unhealthy with specific disease identification within 24 hours, based on biomarker expression allowing early treatment based on clinical chemistry expression.

Regular monthly or bi-weekly blood samples from a large cohort of fish (~30) per site for continuous health monitoring with a report based on the “traffic light” system available within 24 hours via an online portal.

Samples can be collected for sporadic weekly/biweekly diagnostic testing to help identify a specific health challenge.

### Materials and Methods

### Sample Collection & Transportation

### Materials

04.1905.001 S-Monovette 2.6 ml Serum Gel syringes (or 06.1667.001 S-monovette 1.1 ml for smaller fish)
85.1162.200 S-Monovette Safety Needle 21 G×1.5″ (or 85.1441.200 S-monovette safety needle 22 G×1″ for smaller fish) with pre-attached holder & safety device
Rack for syringes
Rack for samples
Pipette and tips
1.5 ml or 2 ml Eppendorf tubes (numbered to ID individual fish)
Centrifuge
Sample submission form
Thermal postal pockets & frozen freezer pack
Aluminium foil

### Method

Fish anaesthetised using MS-222 following manufacturer's instructions.
Whole blood taken from caudal vein, behind caudal fin (FIG. 1) using Sarstedt system.
Insert the needle behind the caudal fin, towards fish spine. Stop when feel resistance, gently pull the plunger until it reaches the end what you can hear as “click” sound. That is the moment when the vacuum is created and at that point you need to find the vein by gently moving the needle to hit the site between the vertebrate under the spine (once you create the vacuum, do not take the needle out). That is why is very important to have deeply anesthetized fish.
Once you hit the spot leave the syringe and allow vacuum to withdraw the blood.
After blood collection, remove needle from the syringe, place cap on needle and dispose of needle in sharp bin provided to you.
Snap the clip of a syringe plunger invert tube ×3 (do not shake) to mix clotting activator and leave standing upright for a minimum of 30 min before centrifugation (Max clotting time 3 h).
After 30 min, centrifuge for 10 minutes (at 3000-4000 g speed).
Allow the centrifuge to come to complete stop. Remove tubes carefully without disturbing contents.
Open the cap of a syringe and pipette serum into labelled Eppendorf tube (ensuring not to disturb the blood clot) make sure that you do not transfer red blood cells from the bottom of tube.
0.3 ml of serum is minimum required for biochemical analysis.
Freeze the samples the same day after collection (−20 °C) and post as soon as possible.
On the day of the postage: Place frozen samples in a bag together with flat freezer pack and wrap the bag in aluminium foil. Pack this into a thermal postal pocket.
Fill in details on Sample Submission Form and include with samples.

### Chemical Chemistry Analysis

### Sample Processing and Analysis

Upon arrival in the lab the frozen samples are defrosted and analysed immediately on the Roche Cobas Pure c 303 analytical instrument. This unit performs more than 110 different clinical chemistry applications including measurements for proteins, enzymes, substrates, electrolytes, drugs of abuse (DATs), and therapeutic drug monitoring (TDMs). The Cobas c 303 has a throughput of up to 450 photometric tests per hour and comes with an integrated ISE unit delivering up to 450 ISE tests per hour. In the mixed mode, the throughput is up to 750 tests/hour (450 ISE+300 photometric tests/hour). The Cobas c 303 has 42 cooled reagent positions.

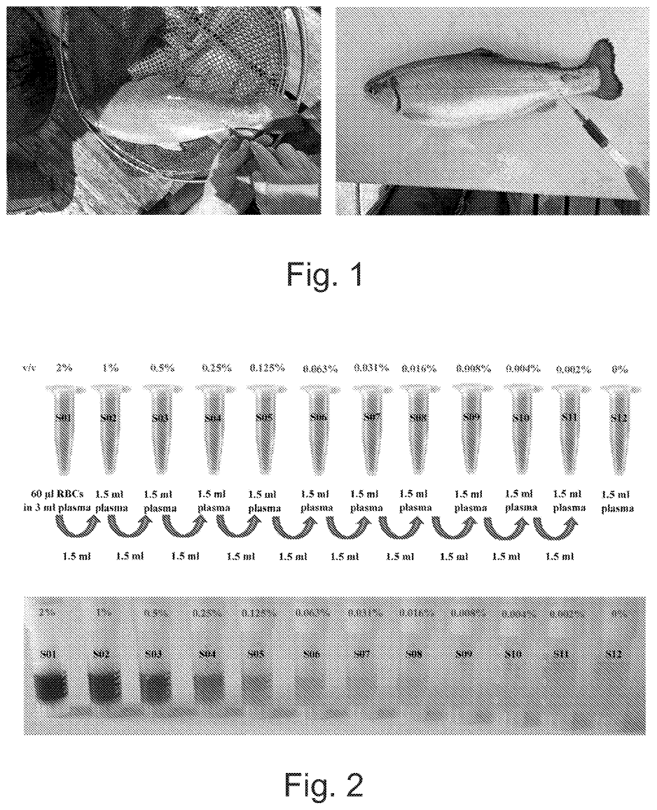

The fresh samples are centrifuged (10 min at 1,200 g) to remove any suspended material, and the supernatant pipetted into the sample cup, observed and given a haemolysis score by comparison to the haemolysis visual chart shown in FIG. 2.

### Clinical Chemistry Measurement Including Optimisation for Fish Blood Serum

The Cobas instrument is operated according to the manufacturer recommendations. The methodology Used is as per assay kit inserts, except 50× dilution for CK and CKMB, and 10× dilution for AST. Other than this the tests are undertaken following the manufacturer's instructions, using all the relevant QC and calibration materials.

The Zinc assay is supplied by Randox on this run on an open channel in the Cobus instrument according to the methodology as per the assay kit insert but through experimentation it was discovered that this assay needs to be run at a 5× dilution.

TABLE 1
Roche Clinical chemistry assays.

| Cat No. | Description |
|---|---|
| ALB2, 750T | Albumin |
| ASTP2, 800T | Aspartate Aminotransferase |
| CA2, 1500T | Calcium |
| ALTP2, 800T, | Alanine Aminotransferase |
| CHOL2, 2600T | Cholesterol |
| CK, 500T | Creatine Kinase - Total |
| CKMB, 150T | creatine kinase-myocardial band |
| GLUC3, 3300T | Glucose |
| IRON2, 700T | Iron |
| LACT2, 100T | Lactate |
| LDHI2, 850T | Lactate Dehydrogenase |
| LIPC, 200T | Lipase |
| MG2, 690T | Magnesium |
| PHOS2, 750T | Phosphate inorganic/ |
| | Phosphorus |
| ISE Probe | Chloride |
| | Sodium |
| | Potassium |

TABLE 2
Randox clinical chemistry assays

| Cat No. | Description |
|---|---|
| ZN2341 | Zinc |

### Example 1: Development of Biomarker Reference Ranges

### Clinical Biochemistry Interpretation Approach

### Introduction

Determine an established background range of 18 biomarkers in farmed salmon across Scotland to allow for clinically relevant interpretation of blood biochemistry results.

### Method

A 1000 salmon blood samples were received from various sites in Scotland. Serum samples were processed on Cobas c303 Roche instrument and 18 biomarkers were measured in each sample as described in the methods above to determine the healthy reference range of the following biomarkers: chloride, sodium, lactate, calcium, magnesium, potassium, alanine aminotransferase, albumin, total cholesterol, creatine kinase, creatine kinase myocardial band, lactate dehydrogenase, aspartate aminotransferase, zinc, iron, glucose, phosphate and lipase (Table 3).

TABLE 3
List of the 18 biomarkers measured using the Cobas instrument
and used to identify fish as healthy or unhealthy and used
to observe the response to various health challenges.

| BIOMARKER | | UNITS | USE |
|---|---|---|---|
| Chloride | Cl | mmol/L | Gills - osmoregulation |
| Sodium | Na | mmol/L | Gills - osmoregulation |
| Lactate | LAC | mmol/L | Gills - oxygen supply |
| Calcium | Ca | mmol/L | Gills |
| Magnesium | Mg | mmol/L | Gills |
| Potassium | K | mmol/L | Gills |
| Alanine | ALT | U/L | Liver biomarker |
| Aminotransferase | | | |
| Albumin | ALB | g/L | Liver biomarker |
| Total Cholesterol | TC | mmol/L | Liver biomarker |
| Creatine kinase | CK | U/L | Skeletal muscle |
| | | | biomarker |
| CK-MB | CKMB | U/L | Heart muscle biomarker |
| Lactate dehydrogenase | LDH | U/L | General health biomarker |
| Aspartate | AST | U/L | Muscle biomarker |
| Aminotransferase | | | |
| Zinc | Zn | μmol/L | General health biomarker |
| Iron | Fe | μmol/L | Anaemia |
| Glucose | GLU | mmol/L | Metabolic stress |
| Phosphate | P | mmol/L | Kidney/Gills biomarker |
| Lipase | LIP | U/L | Pancreas biomarker |

Common descriptive statistics was performed for each biomarker to determine sample size, mean, median, standard deviation, and minimum/maximum values. Next, a test of normality according to Anderson-Darling was executed, with histograms and Q-Q plots for visualisation. Regardless of the results of this test on raw data, the generalized Box-Cox transformation was performed. The 1 and 2 parameters are determined by the maximum likelihood estimator that provides the highest precision.

A Tests of “outliers”: Dixon-Reed and Tukey's tests are then performed. The former detects a single potential outlier based on the ratio of its distance to the nearest value divided by the whole range of values. The latter is based on the median and interquartile range. A box and whiskers diagram (median, 25th and 75th percentiles, 95% 01 of mean), with a dot plot showing all values, is additionally used to visually identify the outliers. In the next step, outliers are removed from the total sample size, and analysis is conducted again starting from normality testing. This procedure is repeated as many times needed till there is no more outliers left per biomarker.

Finally, based on assumptions about data distribution, reference intervals for cleaned series of data are calculated as separate 5 reference intervals. The first 4 intervals are obtained by using standard and robust methods on both non-transformed and transformed data, thus combining the need for data transformation with assumptions regarding Gaussian or symmetrical distribution of data. However, the last (fifth) interval is obtained without any assumption about data distribution. Thus, it is distribution free or equivalently nonparametric. This last interval is used as primary nonparametric reference range for healthy salmon.

In the next step, all 1000 samples are now tested on how well they fit into this primary nonparametric reference range and are labelled (0) if they fit to the range based on all 18 biomarkers or labelled (1) if they fail to fit in with at least one biomarker.

The samples used herein are provided by fish farmers and are accompanied by a sample submission sheet that indicates the health of the fish and identifies any health challenges that have been positively identified using PCR and histopathology. Therefore, we can identify fish coming from a site with a specific health challenge as well as healthy or unhealthy fish. The information provided on these sample submission forms is used to validate the biomarker responses seen in the serum samples. These positively identified fish are used to train the artificial intelligence (AI) models as described below and are also used in blind testing to assess model accuracy.

At this point a more conservative approach is used where the farm score is taken into account. Farmer observations are scored as healthy (0) and unhealthy (1). Where samples that score 0 on the primary nonparametric reference range and score 0 based on farmer observations, a score of 0-0 is given. Only 0-0 matched fish indicating healthy status based on both the reference range and farmer observation from the sample submission forms, enter the database to compute a second reference range.

Once a second reference range is calculated, a final reference range is determined to provide a reference range for each 18 biomarkers. To calculate final reference range, healthy matched fish with a score 0 from both biomarker response and farmers sample submission forms are used, and are cleaned one more time from outliers where everything above mean±2SD is taken away, and what is left is used again to calculate final reference range where a traffic light system is used as follows: healthy reference range average value ±1SD as green, 1-2SD as amber and both together are used to determine healthy reference range. Amber is considered as healthy but a warning that the biomarker is out of the healthy reference range. Everything above or below mean±2SD is considered as unhealthy range, as shown in Table 4.

### Results

Biomarker background levels were determined for each 18 biomarkers analysed in blood samples obtained from farmed salmon. The reference levels of each biomarker was determined for healthy fish, those biomarkers that fell outside of the healthy range but were not within the unhealthy range and unhealthy fish (Table 4).

### Comparison of Healthy Versus Unhealthy Fish

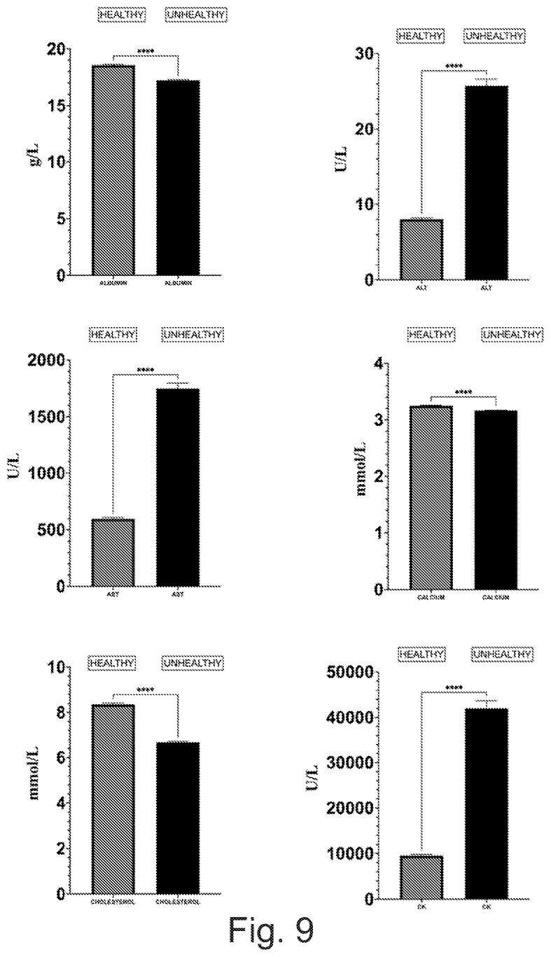

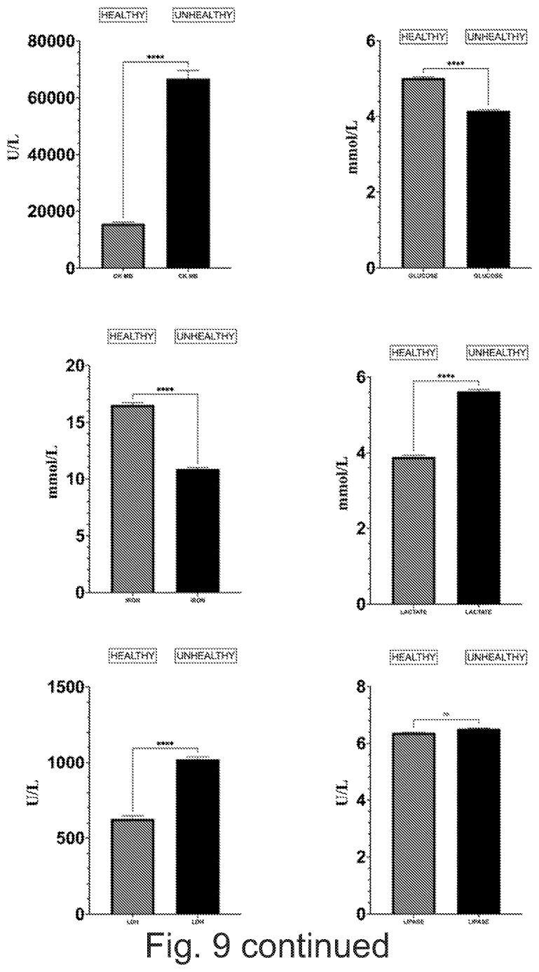

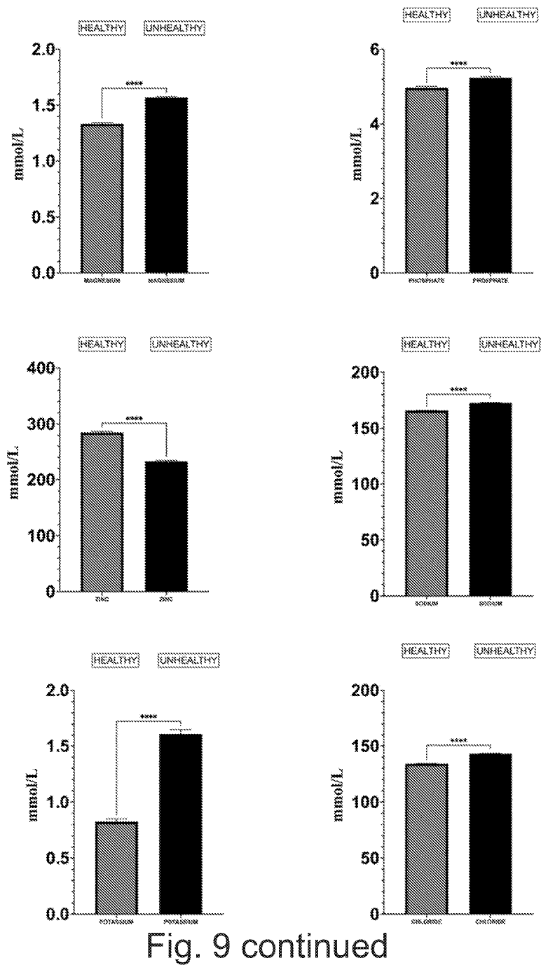

The individual graphs for each biomarker showing the difference in expression between healthy and unhealthy fish is presented in FIG. 9. As indicated by the graph there are significant differences in expression between healthy and unhealthy for the following biomarkers: ALT, AST, TC, CK, CK-MB, FE, K (control healthy fish n=678; unhealthy fish n=3636).

Overall, these results provide the background levels of 18 biomarkers in healthy salmon but also provide a classification system to categorise fish as healthy, unhealthy or provide a warning to fish managers that the fish may becoming unhealthy (warning in the healthy range).

### Example 2: Individual Biomarker Responses to Health Challenge

### Introduction

The present invention provides reference intervals for 18 biomarkers in healthy and unhealthy fish (i.e. a reference profile). The present inventors have then interrogated this data to determine the biomarker expression between fish suffering from different health challenges. In particular, a biomarker expression pattern has been determined for common diseases associated with farmed fish: pancreas disease (PD); cardiomyopathy syndrome (CMS); heart and skeletal muscle inflammation (HSMI); gill issues, gill issues associated with an environmental challenge and bacterial infection. A moribund classification can also be determined which is the terminal stage of any of the challenges described herein.

### Methods

The database consists of 10,000 samples coming from different sites around Scotland. In the database a binary system is used as descriptions of health challenges. Fish get label (+) if the status is positively identified as any one of the following:
Healthy
Pancreas disease (PD)
Cardiomyopathy syndrome (CMS)
Heart and skeletal muscle inflammation (HSMI)
Gill issues
Bacterial infection
Gill issues associated with an environmental challenge (e.g. plankton blooms and/or low oxygen)

Where a sample has a health score 0-0 this means that the sample has been categorised as healthy (0) using the reference range as described above and the farmer score is healthy (0) and a health score of 0-0 is allocated to the sample. Where the sample has been categorised as unhealthy (1) using the reference range as described above and the farmer score is unhealthy (1) then a health score of 1-1 is allocated to the sample. This categorisation is then combined with the labelling as described above for each health challenge.

To determine how each biomarker reacts into each challenge the samples positively identified as unhealthy and with a known health challenge are identified in the database by the following method:
Filter out fish with matched health scores 0-0 and have label healthy (+)
Filter out fish with matched health scores 1-1 and have label for condition PD (+)
Filter out fish with matched health scores 1-1 and have label for condition CMS (+)
Filter out fish with matched health scores 1-1 and have label for condition HSMI (+)
Filter out fish with matched health scores 1-1 and have label for condition Gill issues (+)
Filter out fish with matched health scores 1-1 and have label for condition Gill Issues associated with an environmental challenge (+)
Filter out fish with matched health scores 1-1 and have label for condition Bacterial infection (+)

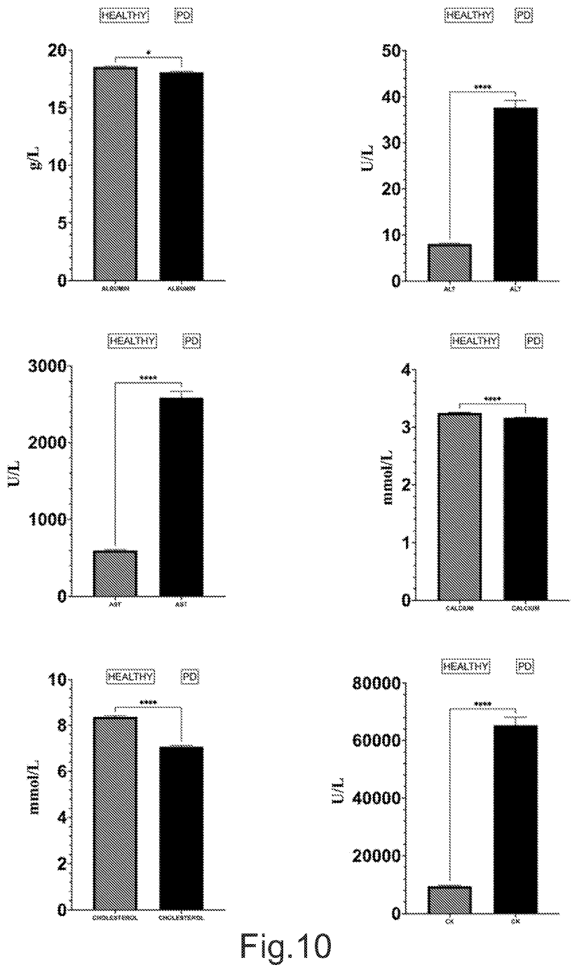

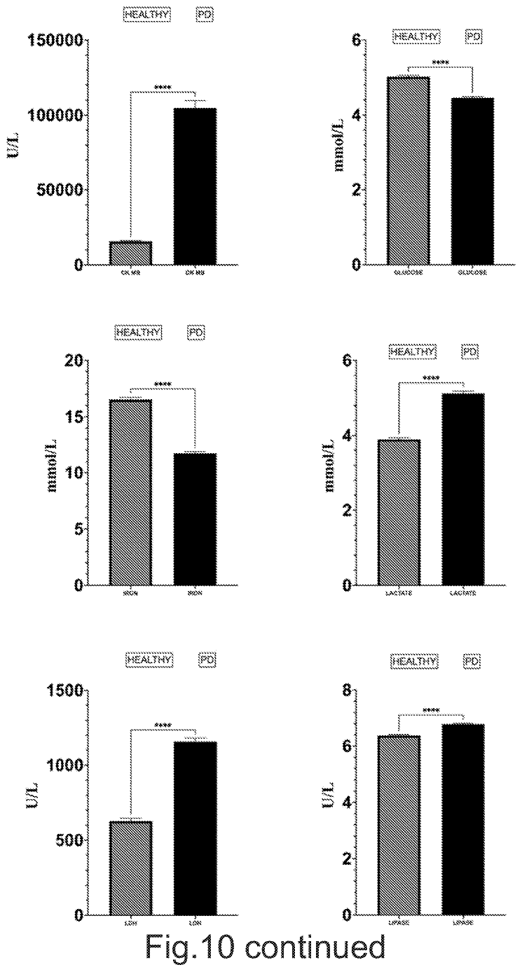

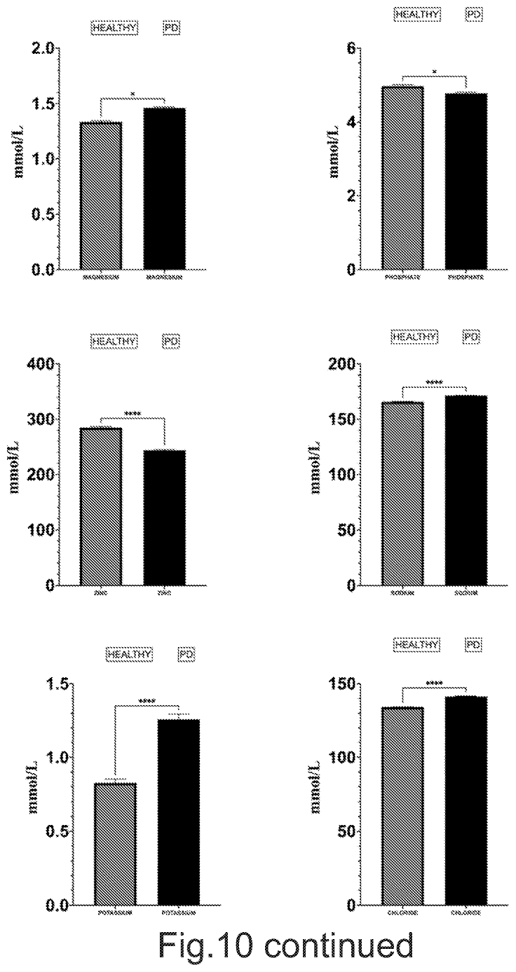

Statistical analysis was then performed where each group of challenged fish are compared against healthy fish. A non-parametric two-sample t-test (Wilcoxon rank-sum), unmatched pairs with statistical differences represented as follows: ns p>0.05, p≤0.05**, p≤0.01***, p≤0.001****, p≤0.0001*****. The biomarker responses (increase or decrease) to each health challenge was then visualised in FIGS. 10-15.

This method is used to draw conclusions on the response of groups of biomarkers, with the patterns resulting from their increase or decrease in expression used to identify the presence of a specific health challenge.

### Results

The individual graphs for each biomarker show the difference in expression between healthy fish and those exposed to PD, Gill Issues, plankton blooms, HSMI, CMS or bacterial infection as presented in FIGS. 10-15 are described in detail for each health challenge below:

### Pancreas Disease (PD)

Samples were collected during PD outbreak and during recovery from PD (n=1997), compared against healthy population (n=678) of fish from monitoring studies.

Biomarkers that showed an increased expression in PD fish included creatinine kinase (CK), creatine kinase-MB (CK MB), Lactate dehydrogenase (LDH), alanine aminotransferase (ALT), aspartate aminotransferase (AST) and Lipase (Lip). Additionally, lactate, magnesium, sodium, potassium and chloride are observed to have increased expression in fish with PD when compared to healthy fish (FIG. 10). Biomarkers with decreased expression in PD infected fish include Total Cholesterol (TC), Glucose (Glu), Zinc (Zn) Phosphate (P) and Iron (Fe) (FIG. 10).

PD recovering fish as used herein may refer to fish that have returned to feeding but still display characteristics of pancreas disease. Suitably, in some embodiments PD recovered fish have increased expression of creatinine kinase (CK), creatine kinase-myocardial band (CK MB), Lactate dehydrogenase (LDH), alanine aminotransferase (ALT) and Lipase (Lip) and decreased expression of Total Cholesterol (TC), Glucose (Glu), Zinc (Zn), Phosphate (P) and Iron (Fe). Suitably, in some embodiments the changes in expression e.g., increased expression of creatinine kinase (CK), creatine kinase-myocardial band (CK MB), Lactate dehydrogenase (LDH), alanine aminotransferase (ALT) and Lipase (Lip) and decreased expression of Total Cholesterol (TC), Glucose (Glu), Zinc (Zn), Phosphate (P) and Iron (Fe) are not to the same extent as PD infected fish, with expression in the “warning” range i.e., amber range (as opposed to the red range).

### Gill Issues

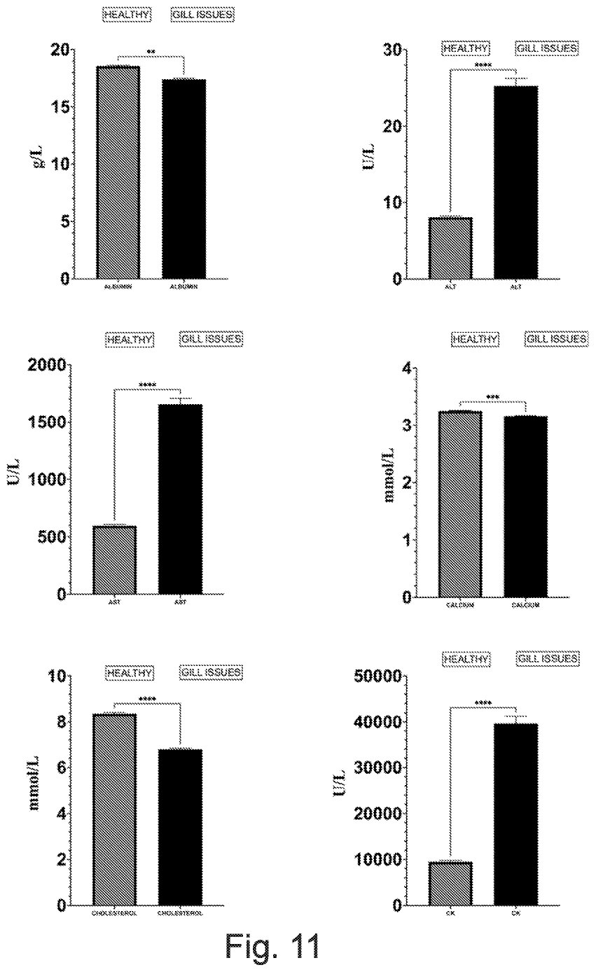

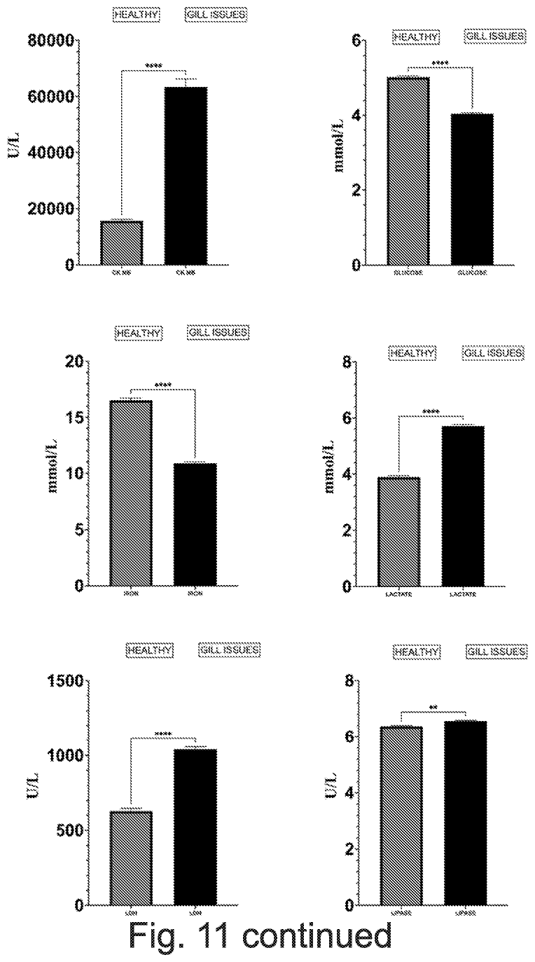

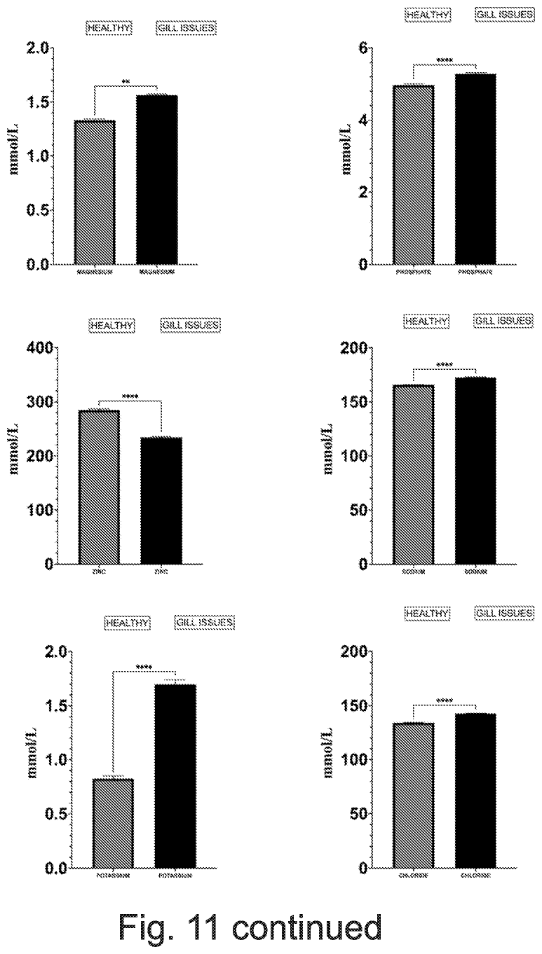

Samples were collected from multiple sampling points with healthy fish (n=678) compared against fish with confirmed gill issues (n=2899). Biomarkers that showed an increased expression in fish with compromised gills include Creatine kinase-myocardial band (CK MB), Lactate dehydrogenase (LDH), Lactate (LACTA), Alanine aminotransferase (ALT), Creatine kinase (CK), and Aspartate aminotransferase (AST), Magnesium (Mg), Phosphate (P), Sodium (Na), Potassium (K), Chloride (CI) and Lipase. Biomarkers with decreased expression in fish with gill issues include calcium (Ca), Albumin (Alb), Zinc (Zn), Iron (Fe), Total cholesterol (TC) and Glucose (Glu) (FIG. 11).

### Plankton Blooms

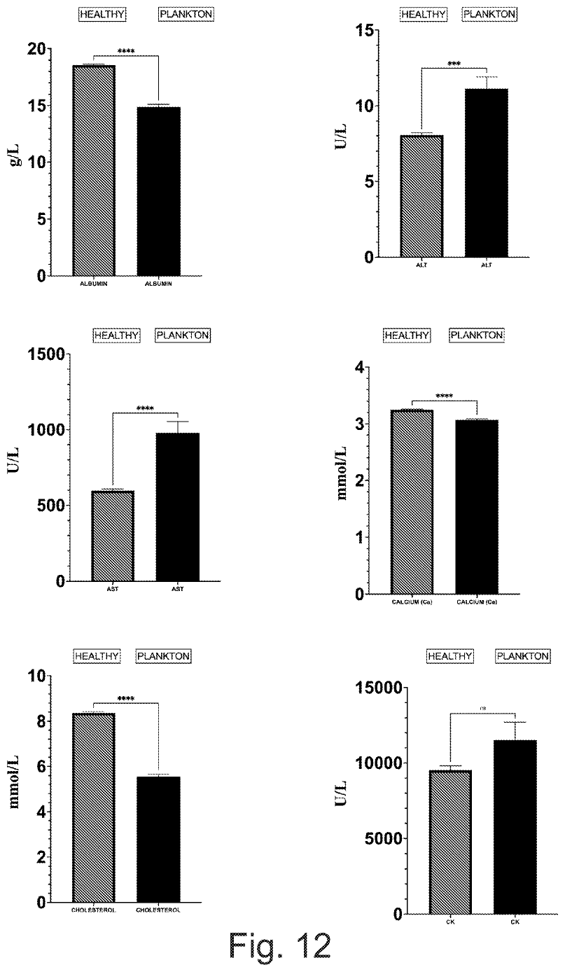

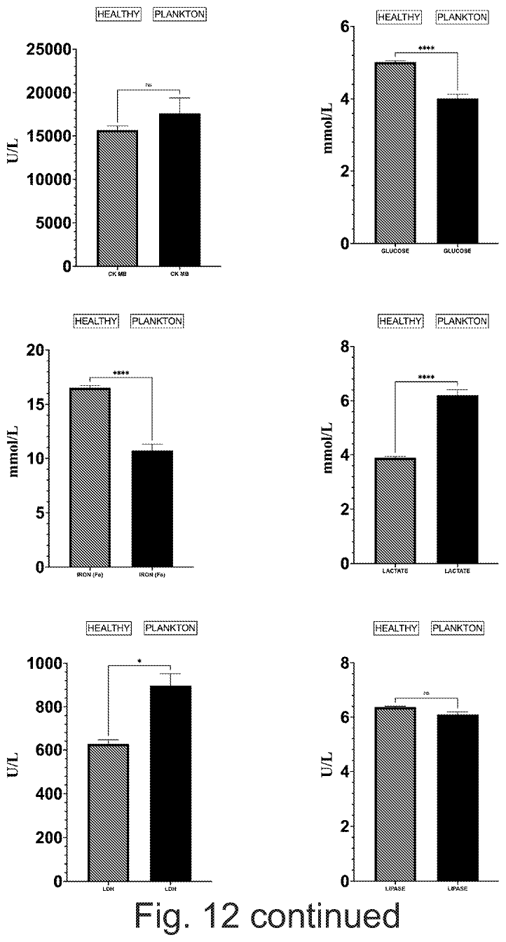

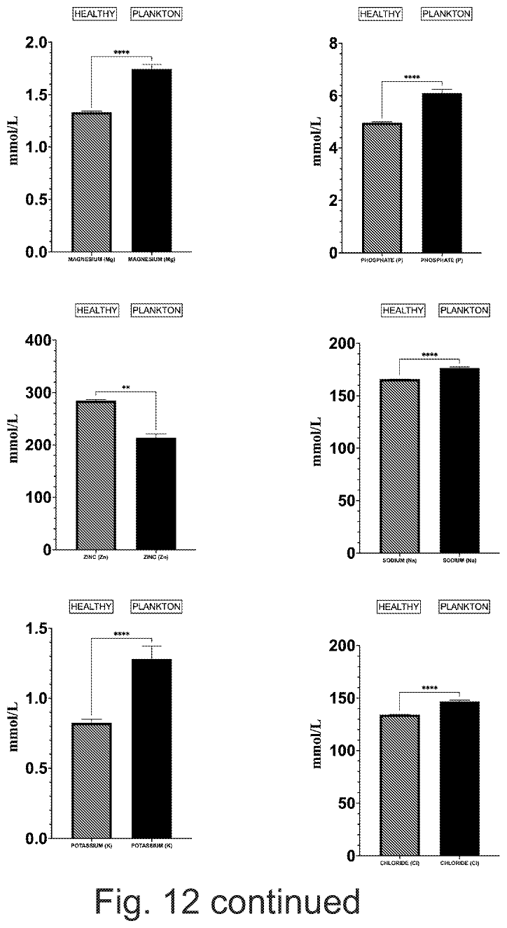

Samples were collected from multiple sampling points with healthy fish (n=678) compared against fish with confirmed exposure to plankton blooms (n=190). Biomarkers that showed an increased expression in fish with exposure to plankton blooms include Creatine kinase-myocardial band (CK MB), Lactate dehydrogenase (LDH), Lactate (LACTA), Alanine aminotransferase (ALT), Creatine kinase (CK), and Aspartate aminotransferase (AST), Magnesium (Mg), Phosphate (P), Sodium (Na), Potassium (K), and Chloride (CI). Biomarkers with decreased expression in fish with gill issues include Albumin (Alb), Zinc (Zn), Iron (Fe), Total cholesterol (TC), Glucose (Glu) and Calcium (FIG. 12). There is no change in the expression of lipase between health and plankton bloom samples (FIG. 12)

### Heart and Skeletal Muscle Inflammation (HMSI)

Samples were collected from multiple sampling points with healthy fish (n=678) compared against fish with confirmed underlying HSMI (n=1522).

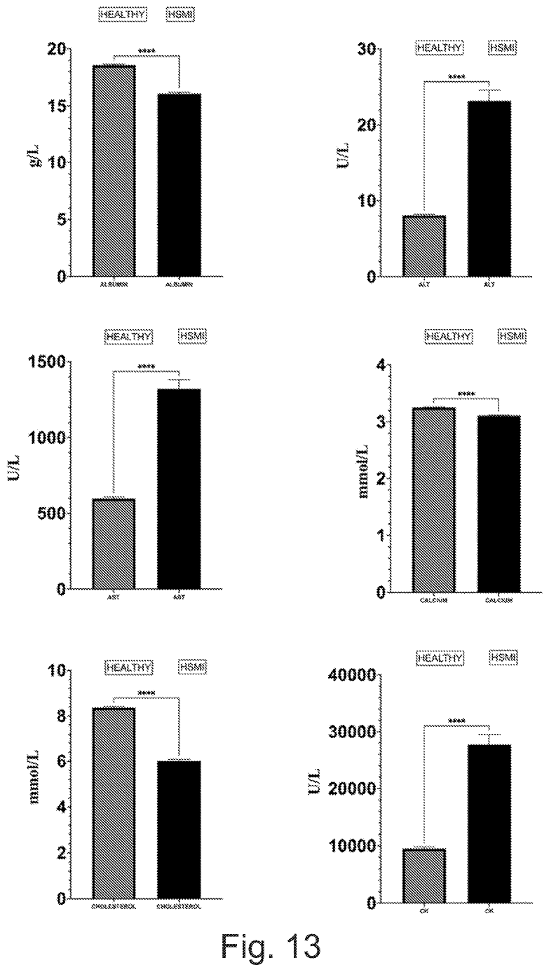

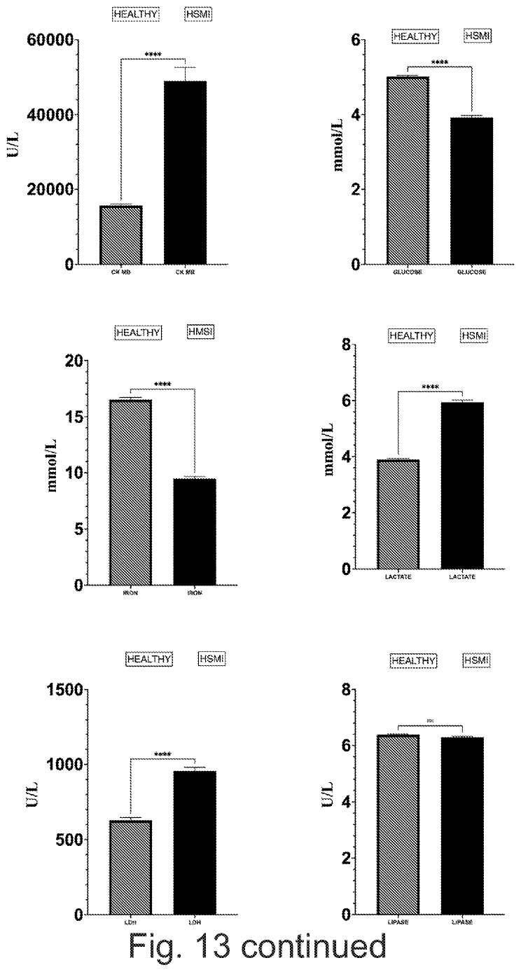

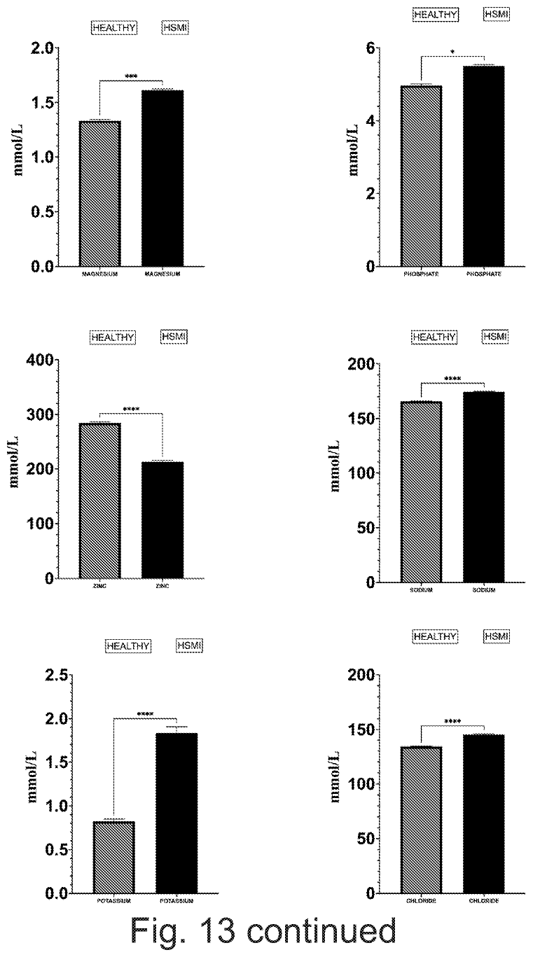

Biomarkers that showed an increased expression in HSMI confirmed fish include Creatine kinase-myocardial band (CK-MB), Lactate dehydrogenase (LDH), Alanine aminotransferase (ALT), Creatine kinase (CK), Aspartate aminotransferase (AST), Magnesium (Mg), Phosphate (P), Lactate (Lacta), Sodium (Na), Potassium (K) and Chloride (CI). Biomarkers with decreased expression in HSMI infected fish include Albumin (Alb), Calcium (Ca), Zinc (Zn), Iron (Fe), Total cholesterol (TC) and Glucose (Glu) (FIG. 13). There is no change in the expression of lipase between healthy and HSMI samples (FIG. 13).

### Cardiomyopathy Syndrome (CMS)

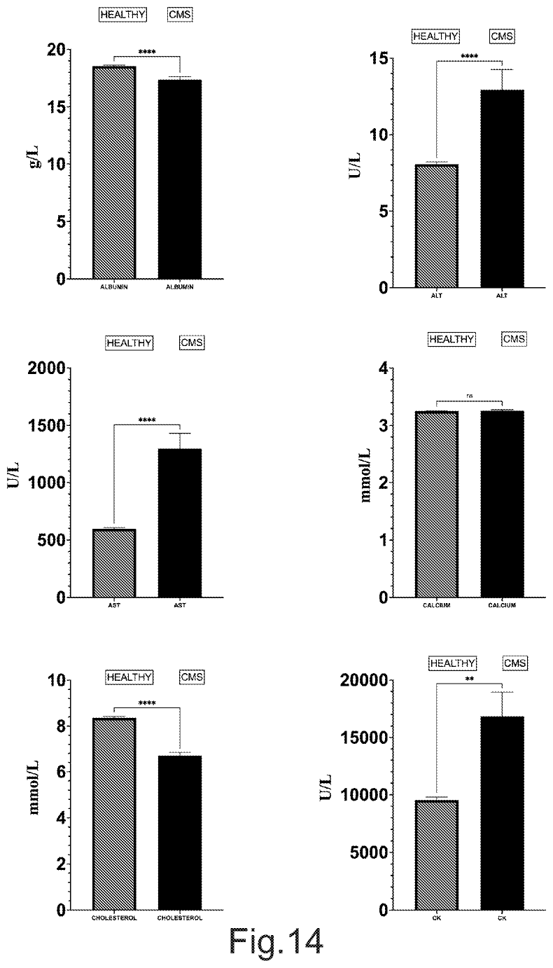

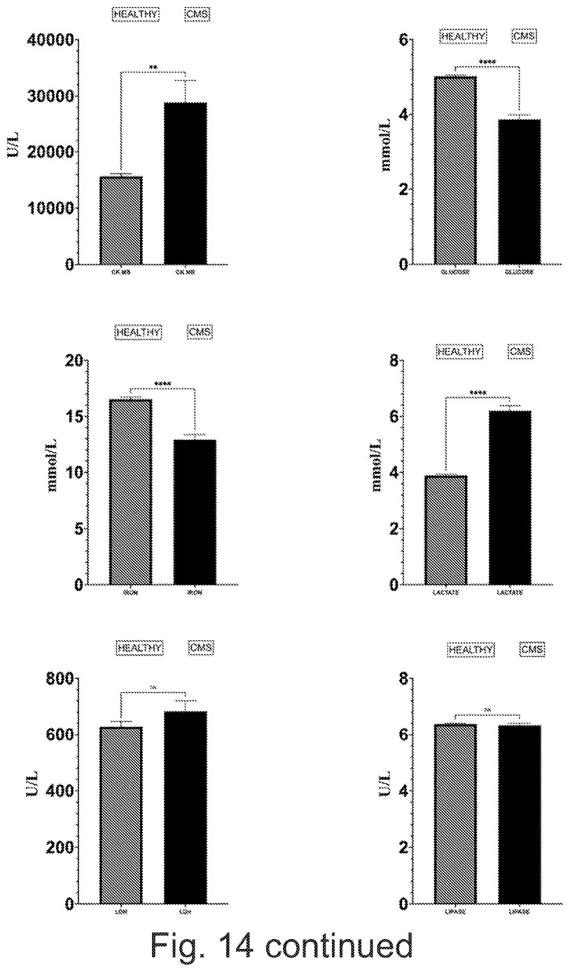

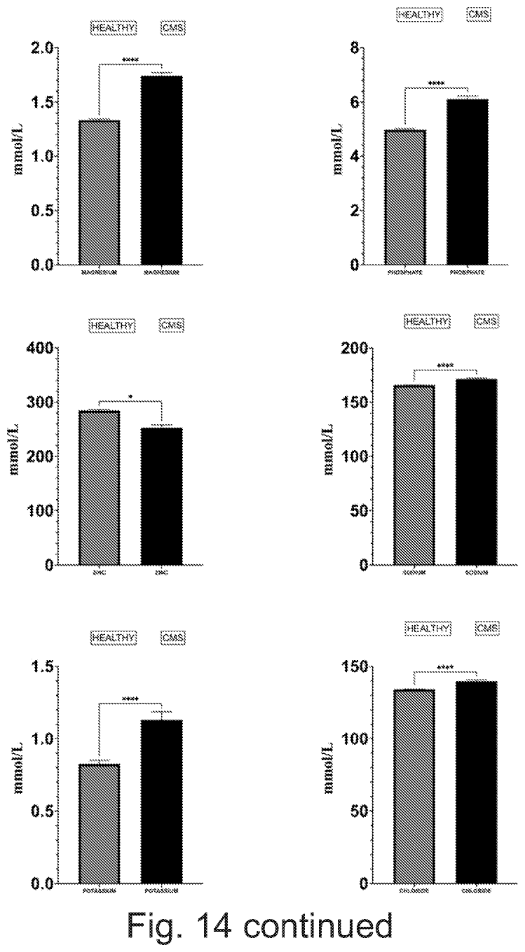

Samples were collected from fish with clinical signs of CMS (n=227) and compared with healthy fish (n=678). Biomarkers that showed increased expression in CMS infected fish include Creatine kinase-myocardial band (CK-MB), Creatine kinase (CK), Aspartate aminotransferase (AST), Alanine aminotransferase (ALT), magnesium (Mg), Phosphate (P), Lactate (Lacta), Sodium (Na), Potassium (K) and Chloride (CI). Biomarkers with decreased expression in CMS infected fish include Albumin (Alb), Zinc (Zn), Iron (Fe), Total cholesterol (TC), and glucose (FIG. 14). There is no change in the expression of calcium, lactate dehydrogenase or lipase between healthy and CMS samples (FIG. 14).

### Bacterial Infection

Samples were collected from multiple sampling points with healthy fish (n=678) compared against fish with confirmed bacterial challenge (n=600). Most common bacterial challenges in aquaculture include Aeromonas salmonicida, Vibrio species such as Vibrio anguillarum and Vibrio salmonicida, Yersinia ruckeri, Pasteurella skyensis, Tenacibaculum maritimum, Flavobacterium psychrophilum and Piscirickettsia salmonis.

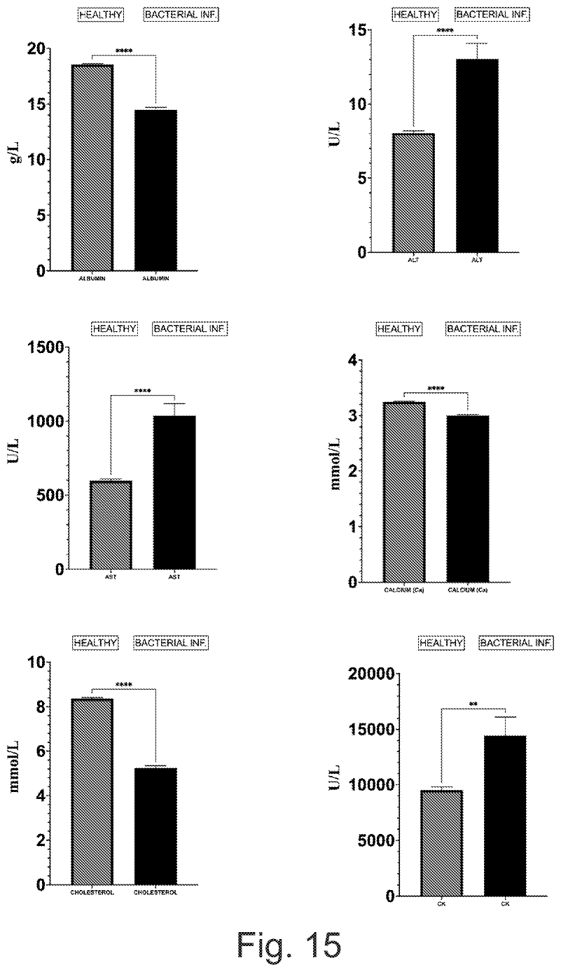

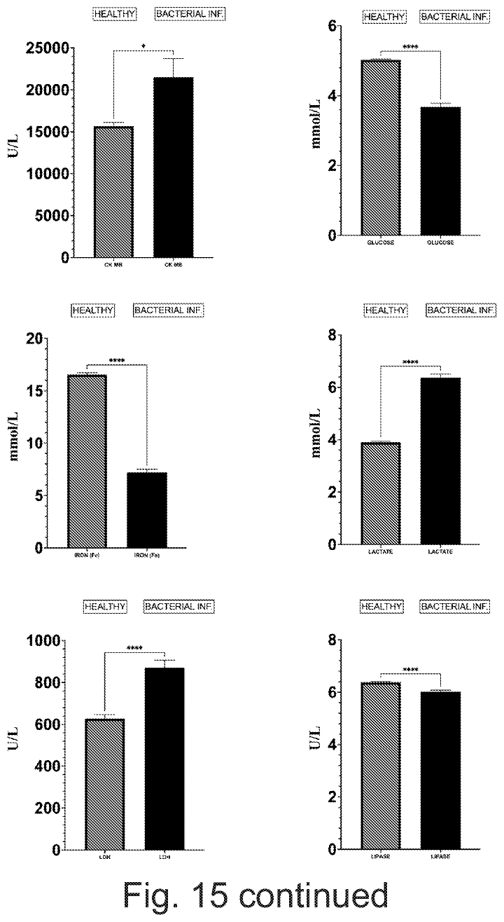

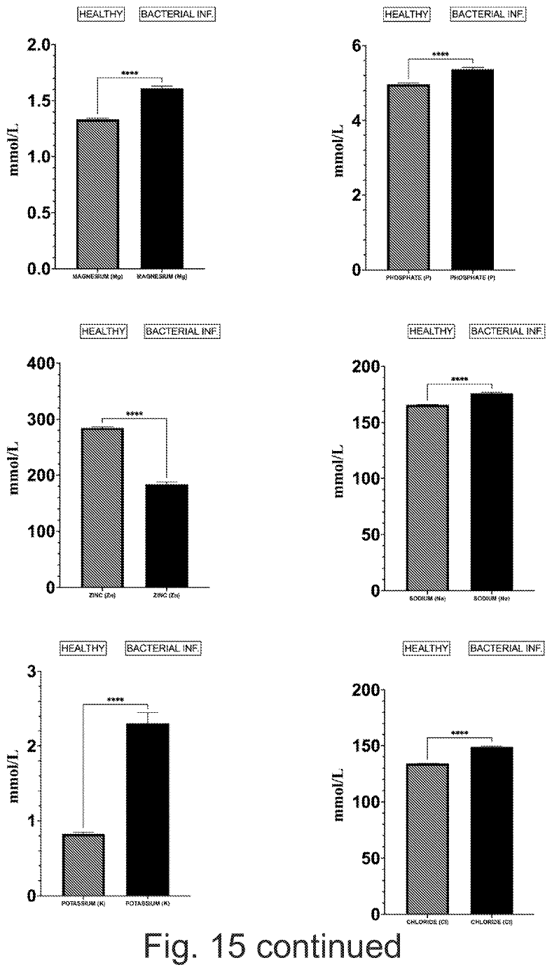

Biomarkers that showed an increased expression in bacterial infected fish include LDH, LACTA and CL. Additionally, an increase in ALT, AST, CK, CK-MB, Mg, P, Na and K are also increased in fish with bacterial infection. Biomarkers with decreased expression in bacteria infected fish include Alb, Zn, Fe, TC, CA, GLU and LIP (FIG. 15).

The relative changes in biomarker expression for each of the health challenges when compared to healthy are shown in table 5. For example, fish suffering from gill issues show an increase in ALT, AST, CK, CK-MB, LACTA, LDH, MG, P, NA, K and CL and a decrease in ALB, CA, TC, GLU, FE and ZN.

Samples obtained from fish that are near death are classified as moribund. This is the terminal state of any of the health challenges as described herein and may be identified from the biomarker response as indicated in Table 5.

### Example 3: Artificial Intelligence Model Development

### Introduction

To aid data interpretation and improve the efficiency of data analysis, the present inventors have developed an AI model to categorise the fish as healthy versus unhealthy (model 0) and for the unhealthy fish to further categorise the specific health challenge based on the biomarker expression (model 1).

### Method

The AI model-based system conception is outlined in FIG. 2.

### Dataset:

The dataset used for building the AI models is a real-world dataset that stemmed from the data collected from samples sent to the laboratory by various locations around Scotland and were accompanied with a sample submission form indicating the health of the fish as recorded by the farmers, with all health challenges having been identified by PCR or histopathology as appropriate (similar to the data set used above). Samples were analysed using the Cobas instrument, and the clinical chemistry data generated was examined by a biochemistry expert. As described above, only data that had the same health status as identified by the farmer and biochemistry expert was used to train the model (i.e. 0-0 or 1-1). The dataset consists of 18 biomarkers as described in Table 3. This dataset is used to diagnose fish health and challenges from different locations around Scotland. The challenges covered by our models are CMS, Gill Issues, PD and HSMI. Due to initial low sample numbers, analysis of samples from bacterial infection and gill issues associated with an environmental challenge are ongoing as sample numbers accumulate. To this dataset, we applied pre-processing techniques including missing, duplicated values, outlier detection and imbalanced data handling.

### Missing Value Detection

This step consisted of identifying and dealing with the missing value. For most of the missing values, the data was deleted followed by a deep discussion with the experts regarding these missing values. In fact, filling through some imputation techniques may not be appropriate in our case as these missing values are those with negative values (these values are currently out of the analysis from an expert) provided by Cobas.

### Outliers Detection

As a technique, the isolation forest was used to identify and drop the dataset outliers. Isolation Forest is based on the Decision Tree algorithm. It isolates the outliers by randomly selecting a feature from the given set of features and then randomly selecting a split value between the max and min values of that feature.

### Imbalanced Data Handling

Our classification models were challenged by an imbalanced dataset that affected our initial models performance. In order to overcome that, SMOTE (for model0) (Chawla et al. 2002; (SMOTE-Python v3.8.13, Version 0.11.0, n.d.-b)) and MLSmote (for multi-label for model1) (Charte, 2015) (Niteshsukhwani, n.d.) (techniques were introduced to balance the dataset.

### Libraries Utilised in AI Model Development

TABLE 6
libraries of importable code utilised in
AI model development and version numbers.

| Library | Version |
|---|---|
| BorutaShap | v1.0.16 |
| explainerdashboard | v0.4.0 |
| imbalanced-learn | v0.9.1 |
| matplotlib | v3.5.2 |
| numpy | v1.25.0 |
| pandas | v2.0.3 |
| shap | v0.41.0 |
| scikit-learn | v1.1.1 |
| skmultilearn | v0.20 |
| Python | v3.8.13 |
| pickle | (from python v3.8.13) |

### Model Development

### Model0 (Healthy Vs Unhealthy) Based on a Single Random Forest Learner

A tailored random forest classifier was trained on balanced data based on Smote technique and improved via GridSearch learner to ensure a health diagnostic prediction above 96%. The dataset was trained on data whose health scores are the same for both farmer and biochemistry experts as mentioned above. In order to ensure higher and more reliable performance of model0, a stratified K-Fold cross-validation method was applied to evaluate and test model0 performance. It provides the ability to estimate model performance on unseen data not used while training. Cross-validation is a technique for evaluating machine learning (ML) models by training several ML models on subsets of the available input data and evaluating them on the complementary subset of the data.

### Model1 (Challenge Prediction) Based on Stacked Random Forest Leaners

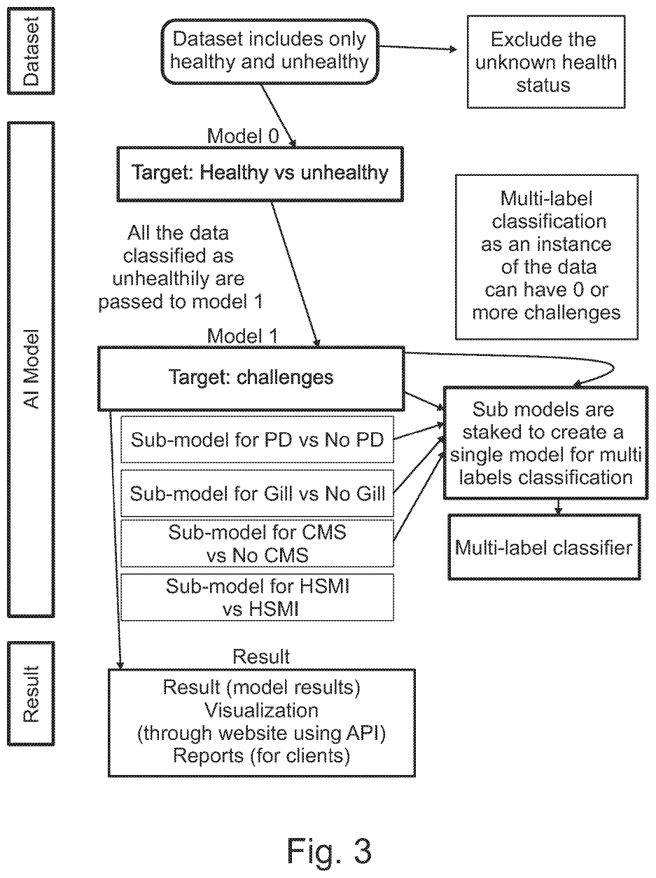

The model1 is a tailored model based on an ensemble model. This ensemble model consists of a sub-model based on random forest learners used to train and cover the challenges. As described in the FIG. 3 related to the model conception, the unhealthy dataset is used by four sub-models based on random forest and respectively used to cover four challenges (Gill issues, CMS, PD, HSMI). Following this, the ensemble learner is used to form a stacked model based on four trained sub-random forests and trained on all unhealthy datasets.

Furthermore, as multi-label classification was used (because a fish can have one or more challenges), a tailored and improved PowerlabelSet derived from GridSearch was used to cover the multi-label classification for the fish health challenges. This multi-label classifier (PowerLabelSet) was used as the base classifier (our formed ensemble classifier) to make the challenge prediction (see FIG. 3). Cross-validation was applied to our model1 to evaluate and test its performance as explained above in model0 section

### Key Biomarkers for Health and Challenges Prediction

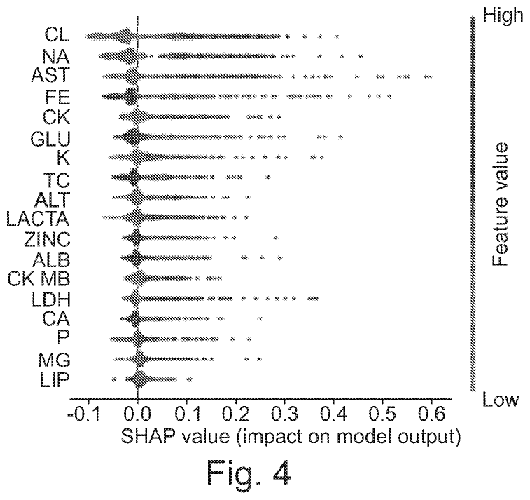

To determine key biomarkers that determine the fish health and challenge predictions using model0, the rank of the biomarkers was investigated in the Shap summary plot (FIG. 4) that provides the importance of the biomarkers for a given model applying to a dataset. Progressively, the number of biomarkers was reduced while ensuring an acceptable model performance >80%. Finally, the key biomarkers are selected by comparing and selecting the top biomarkers present in the models progressively built (Table 7).

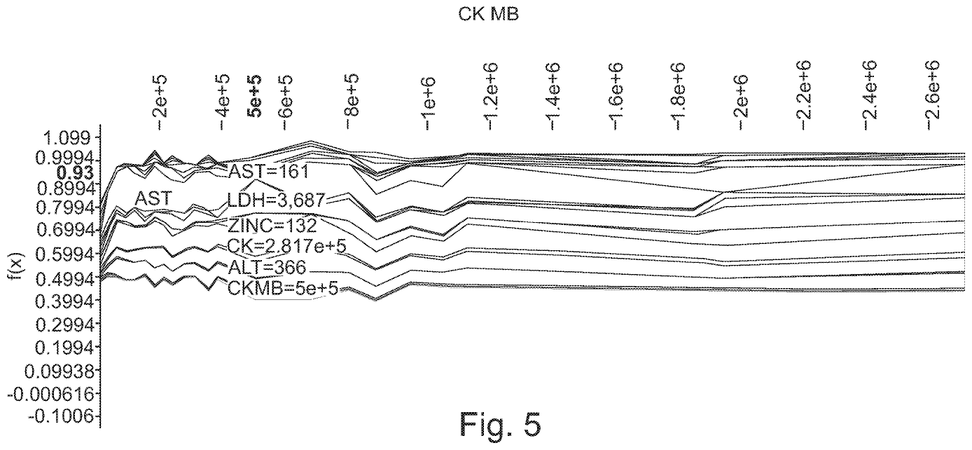

Furthermore, for each biomarker their threshold value was identified that impacts the prediction of models (model0 and model1). These values were identified by observing the Shap force plot (FIG. 5) of a prediction of the models (model0 and model1) for a dataset.

Indeed, the Shap force plot as shown in FIG. 5 allows us to observe a value range of the features (biomarkers in this case) that impact the model prediction. For example, as shown the FIG. 5 for a fish with a value of 500,000 IU/I of OK MB, the model will likely classify this fish as an unhealthy fish. However, the skilled person will understand that it is the interaction of the biomarkers that influence the prediction and a typical value such as 500,000 IU/I of OK MB is not to be considered in isolation.

### Results

### Model0 (Healthy Vs Unhealthy) Based on a Single Random Forest Learner

A dataset of 6744 entries was first split into the training and testing sets with a test size equal to 0.2, and then the SMOTE and Tomek links. A combination of oversampling and under sampling method was employed on the training data to construct synthetic instances to deal with imbalance data by getting a size of 50%:50% for both classes. For the classification, a random forest algorithm was applied to build predictive models. In the training process, StratifiedKfold (10-fold) cross-validations were performed to search for optimal hyperparameters. Stratified K Fold was implemented to reduce or remove bias in the model created by the health class prediction. The classifiers with the best parameters were fitted into the training data to obtain a model (model0) and then test on the remaining 20% of data. The performance of this model is compared with the following criteria: precision, recall, accuracy and F1-score. To determine the key biomarkers, the above process has been applied to model0 where the number of biomarkers was progressively reduced from 18 biomarkers to 15, 12 and 7 biomarkers (model18, model15, model12 and model7).

As a result, the performance of model0 with different numbers of biomarkers was calculated (Table 8). Different model0's were produced with performance higher than 90% as shown the Table 8 providing the details of the precision and recall of each model0. By comparing the model0 performances, the model0 with 18 biomarkers ensures better (above 96%) health diagnostic prediction than the other model0s. For example, this table shows that model0 has a precision of 0.97 and a recall of 0.97. In other words, when model0 predicts a fish is unhealthy, it is correct 97% of the time and it correctly identifies 97% of all unhealthy fish.

TABLE 8
Model 0 precision and sensitivity (training and unseen data using cross validation).

| | Model 18 | | Model 15 | | Model 9 | | Model 7 | |
|---|---|---|---|---|---|---|---|---|
| | Precision | Recall | Precision | Recall | Precision | Recall | Precision | Recall |
| Unhealthy | 97% | 97% | 97% | 97% | 97% | 95% | 97% | 94% |
| Healthy | 80% | 80% | 76% | 80% | 70% | 80% | 61% | 77% |

For validation purposes, the model was run on a different test dataset. The results of model0's performance are summarized the in Table 9.

TABLE 9
Model0 with 18 biomarker performances from dataset summary
(testing model with unseen data without cross validation).

| | Data from 18 Sep. 2022 | | Data from 2 Nov. 2022 | |
|---|---|---|---|---|
| Correct ‘healthy’ | 69 | (70%) | 27 | (88%) |
| prediction | | | | |
| Incorrect ‘healthy’ | 19 | (30%) | 6 | (22%) |
| prediction | | | | |
| Correct ‘unhealthy’ | 672 | (97%) | 920 | (99%) |
| prediction (based on | | | | |
| expert diagnostic) | | | | |
| Incorrect | 14 | (3%) | 1 | (1%) |

‘unhealthy’
prediction

To further assess the performance of model0, the health assessment prediction by the model was compared with that of the biochemistry expert (Table 10).

### Model1 (Challenge Prediction) Based on Stacked Random Forest Learner

The current model1 ensures a prediction of multi-challenge prediction with a hamming loss lower than 0.15. Table 11 summarizes the confusion matrix of the current model. As shown in the result, the precision and sensitivity related to the classification of this model are on average over 80%.

Model1 was trained on unhealthy fish only based on an unhealthy fish dataset by comparing between one health challenge and another health challenge. This is different to the data presented in FIGS. 10-15 where each challenge is compared against the healthy fish. For this reason the results generated by the two methods do not match and can identify differences in biomarker expression between the two methods.

TABLE 11
Summary of the confusion matrix of the current model.

| | precision | recall | f1-score | support |
|---|---|---|---|---|
| 0 | 0.87 | 0.97 | 0.92 | 447 |
| 1 | 0.90 | 0.80 | 0.85 | 116 |
| 2 | 0.80 | 0.84 | 0.82 | 275 |
| 3 | 0.74 | 0.68 | 0.71 | 206 |
| micro avg | 0.83 | 0.86 | 0.85 | 1044 |
| micro avg | 0.83 | 0.82 | 0.82 | 1044 |
| weighted avg | 0.83 | 0.86 | 0.84 | 1044 |
| samples avg | 0.84 | 0.87 | 0.84 | 1044 |

### Feature Importance

The Shap values of the biomarkers for model0 and model1 were evaluated through a dataset samples used for the test. Table 12 shows the key biomarkers for the prediction (health and challenges) according to the model0 and model1's current performances.

The key biomarkers from model0 to distinguish between healthy and unhealthy fish and how each biomarker can affect the model's prediction is shown in Table 12. The values in this table are the estimated ones identified through the visualisation of the Shap force plot of the models predictions. The values shown are exemplary and do not provide a distinct cut off but provide an exemplary scenario of when the model would predict a healthy fish or an unhealthy fish (Table 12).

The key biomarkers from model1 to distinguish between different health challenges and how each biomarker can affect the model's prediction is shown in Table 12. The values in this table are the estimated ones identified through the visualisation of the Shap force plot of the models predictions. The values shown are exemplary and do not provide a distinct cut off but provide an exemplary scenario of when the model would predict where an unhealthy fish has gill issues, CMS, PD or HSMI (Table 13).

### AI Model Discussion

Two AI models were built and trained for supporting fish health and challenges diagnostics. As result, model0 can ensure prediction above 96% accuracy to determine if a sample is from a healthy or an unhealthy fish, thus demonstrating an accurate and robust model for fish health prediction. Further analysis of model0 revealed the key biomarkers based on feature importance from the Shap framework. It was observed that the biomarkers can slightly change through different tests involving different datasets. However, the top 7 biomarkers in the Table 12 have not changed through the tests. A Model0 has been trained from these 7 biomarkers and results show the ability to predict the fish health with performance over 90%.

Model1, was more challenging due to multi-label classification compared to the binary classification of model0. However, by looking at the hamming loss value of model1 which is less than an average of 0.3 after running 2 datasets, the current model performance is acceptable as this hamming loss shows a lower prediction error (an incorrect label is predicted). From this current model, we were able to identify, a list of key biomarkers (based on feature importance) for each of PD, Gill Issues, HSMI and CMS. As the dataset for model1 grows the performance will improve. It will be readily understood that model1 can also classify the challenges of bacterial infection and gill issues associated with an environmental challenge by further training model1 with known bacterial infection samples and samples from fish exposed to an environmental challenge such as plankton blooms and/or low oxygen levels.

### Example 4: Determining a Welfare Score Based on Single Prediction Probability from Model0

### Introduction

The methods thus far provide at an individual level or population level the ability to determine the health status of fish and to further diagnose or predict if unhealthy fish have a particular health challenge. However, the reality of farmed fish populations are large numbers of fish housed together in pens, with multiple pens across a site. Therefore, developing a welfare score to indicate the overall welfare of a fish population based on the cumulative health score of individual fish is necessary for fish farm managers to understand the health and welfare of their fish at a site wide population level.

### Methods

In order to determine the welfare score of a set of fish serum samples (954 samples) collected from a sampling site. To determine a welfare score, a further model, Model2, was developed which consists of 2 subsystems: system1 and system2. System1 is an algorithm that classifies fish based on predefined health levels built from fish health prediction probabilities from Model0. System2 calculates the ratio of each health level(healthy ratio, weak health ratio, unhealthy ratio) from the welfare score provided by system1 for the given population. Based on prediction probability ranges assigned to health levels, system1 identified the health level for a given fish belonging to a fish set by evaluating the health prediction probability generated by Model0 for this fish. This classification process was applied to the fish dataset for which health diagnostics was initially predicted. Then the welfare score ratio of this dataset was determined by calculating the ratio of each health level in the fish dataset.

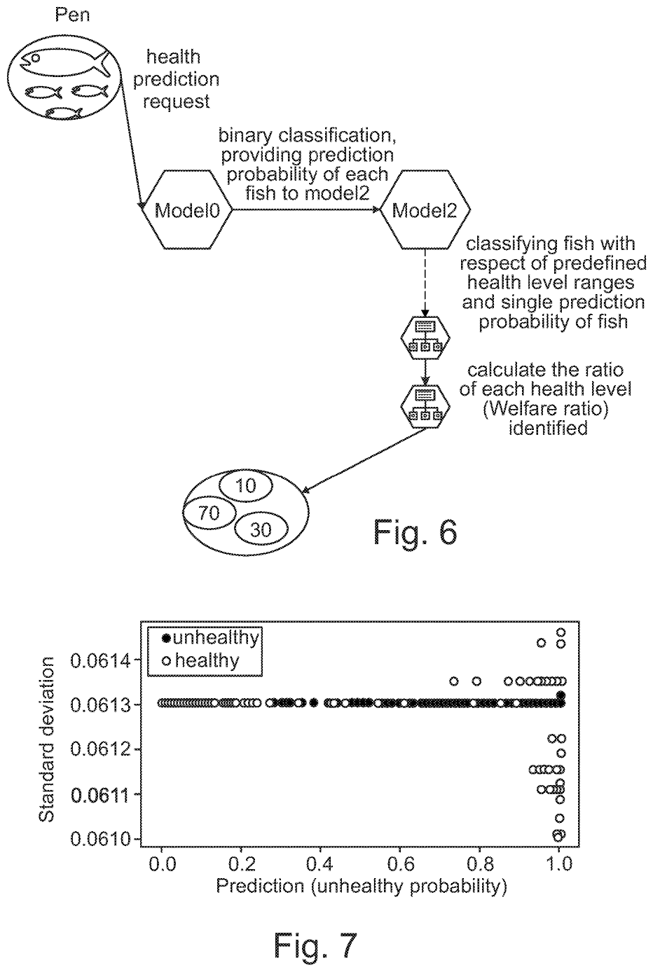

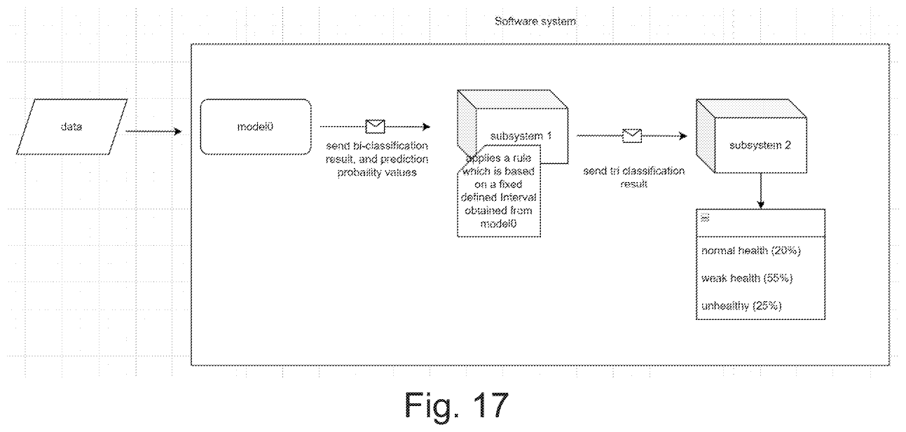

The process used to determine the fish welfare score is as follows and is summarised in FIG. 6 and FIG. 17:
1—Fish dataset was passed through model0 to predict health status (i.e. binary classification of healthy or unhealthy) and the single probability prediction of each fish was directed to model2
2—Model2 subsytem1, based on the predefined ranges of health levels, classified every single fish into a specific health level category
3—Then Model2 subsystem 2 parallelly calculates the ratio of fish belonging to each health level

### Predefined Health-Level Range Construction

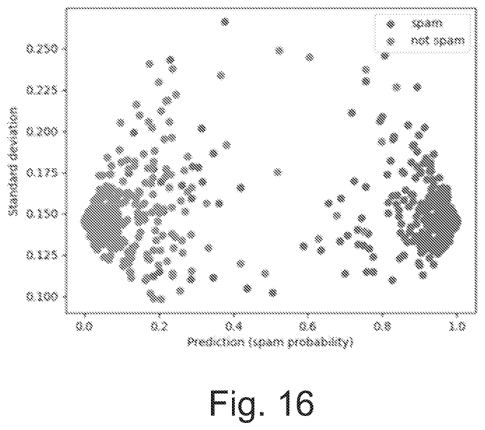

As described above, the set of interval ranges is defined by those that correspond to the ranges in which the fish health level fall in. The Fish health levels have been grounded built by looking at the distribution of probabilities of single prediction from a model0 for test dataset(s) as shown in the FIG. 7. To validate the selected level ranges, the models confidence in the prediction probability (or prediction) of each fish was tested. Model0 with the highest prediction performance was used for model2. The model with the highest confidence about its prediction was selected. By that it is meant that the confidence interval for predictions should be lower (that is SD of each prediction should be in a smaller interval). For example, where the SD interval of an entire set prediction belongs to 0.1 0.2550 (FIG. 16) and another SD of entire set prediction belongs to 0.002, 0,005, the latter model will be preferred than to the previous one. These confidence intervals can be calculated with the following package: (Python v3.8.13, Forestci API—Forestci 0.6 Documentation, n.d.).

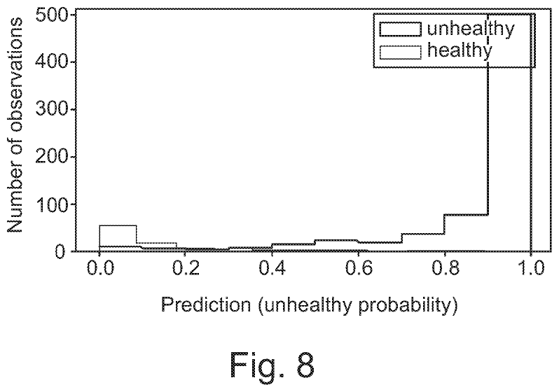

Therefore for a given probability distribution obtained from the mode0 on a test dataset X as shown in the FIG. 8 the following health intervals are defined:
Good health: define the highest level of the healthiness of a fish. Any prediction probability corresponding to this level should fall into the prediction probability value close to 0
Weak health: define an acceptable level of the healthiness of a fish. Any prediction probability corresponding to this level should fall into the prediction probability value close to the prediction cut-off value
Critical health: defined as unhealthy fish. Any prediction probability corresponding to this level should fall into the prediction probability value close to 1

As result we can define the following Welfare intervals:
Good health: [0.1-0.3]
Weak health: [0.3-0.7]
Critical health: [0.7-1]

### Results

As mentioned in the methodology, a suitable model is one whose welfare score classification of subsystem1 for a specific data set is the same as that of the biologist's diagnosis.

The results of model2 (subsystem1 (welfare score) without subsystem2 (population welfare value) for a dataset (derived from table 8) are shown in Table 14. the dataset has been classified into 3 categories by model2 to determine a population welfare score:
28 unhealthy fish (80%)
4 fish with normal health (11%)
3 fish with weak health (9%)

This classification as shown in the table is approximatively aligned with the biologist diagnosis.

The fish predicted as unhealthy by the biologist are also considered as unhealthy by the model2. A similar result was also obtained with healthy fish to the healthy fish (Table 14). The other fish predicted with weak health by model2 does not reject the fact that a fish is only healthy or unhealthy, but considers the risk that the fish can be healthy but present also an unhealthy state vis-versa, therefore, model2 does not reject the biologist diagnoses a shown the Table 14.

### REFERENCES

1. Adel, M., Abedian Amiri, A., Zorriehzahra, J., Nematolahi, A. & Esteban, M. A. 2015. Effects of dietary peppermint (Mentha piperita) on growth performance, chemical body composition and hematological and immune parameters of fry Caspian white fish (Rutilus Frisii Kutum). Fish Shellfish Immunol. 45(2), 841-7.
2. Barisic, J, Cannon, S., Quinn, B. 2019. Cumulative impact of anti-sea lice treatment (azamethiphos) on health status of Rainbow trout (Oncorhynchus mykiss, Walbaum 1792) in aquaculture. Scientific Reports volume 9, Article number: 16217.
3. Benfey, T. J. & Biron, M. 2000. Acute stress response in triploid rainbow trout (Oncorhynchus mykiss) and brook trout (Salvelinus fontinalis). Aquaculture 184, 167-176.
4. Bernet, D., Schmidt-Posthaus, H., Wahli, T. & Burkhardt-Holm, P. 2000. Effects of wastewater on fish health: an integrated approach to biomarker responses in brown trout (Salmo trutta L.). J. Aquat. Ecosyst. Stress Recover. 8, 143-151.
5. Braceland, M., Houston, K., Ashby, A., Matthews, C., Haining, H., Rodger, H., Eckersall, P. D. 2017. Technical pre-analytical effects on the clinical biochemistry of Atlantic salmon (Salmo salar L.). Journal of Fish Diseases 2017, 40, 29-40.
6. Charte, F. R. (2015). MLSMOTE: Approaching imbalanced multilabel learning through synthetic instance generation. Knowledge-based systems, 385-397.
7. Nitesh V Chawla, Kevin W Bowyer, Lawrence O Hall, and W Philip Kegelmeyer. Smote: synthetic minority over-sampling technique. Journal of artificial intelligence research, 16:321-357, 2002.
8. FAO 2020: The State of World Fisheries and Aquaculture 2020 report by the United Nations Food and Agriculture Organisation (UN FAO).
9. Breiman, Leo. Random forests. Machine learning 45.1 (2001): 5-32.
10. Devetyarov, D., and Nouretdinov, I. 2010. Prediction with confidence based on a random forest classifier. IFIP International Conference on Artificial Intelligence Applications and Innovations. Springer, Berlin, Heidelberg.
11. Ferri, J. et al. 2011. The effect of artificial feed on blood biochemistry profile and liver histology of wild saddled bream, Oblada melanura (Sparidae). Mar. Environ. Res. 71, 218-224.
12. Floyd-Rump, T. P., Horstmann-Dehn, L. A., Atkinson, S. & Skaugstad, C. 2017. Effect of ichthyophonus on blood plasma chemistry of spawning chinook salmon and their resulting offspring in a yukon river tributary. Dis. Aquat. Organ. 122(3), 223-236.
13. Feurer, Matthias, et al. Auto-sklearn (2018): efficient and robust automated machine learning. Automated Machine Learning: 113-134.
14. Hatami, Nima, and Reza Ebrahimpour. 2007. Combining multiple classifiers: diversify with boosting and combining by stacking. International Journal of Computer Science and Network Security 7.1: 127-131.
15. Hille, S. 1982. A literature review of the blood chemistry of rainbow trout, Salmo gairdneri Rich. J. Fish Biol. 20, 535-569.
16. Javed, M., Ahmad, M. I., Usmani, N. & Ahmad, M. 2017. Multiple biomarker responses (serum biochemistry, oxidative stress, genotoxicity and histopathology) in Channa punctatus exposed to heavy metal loaded wastewater. Sci. Rep. 7, 1765.
17. Marco Rozas-Serri, R. C.-V. (2022). Reference Intervals for Blood Biomarkers in Farmed Atlantic Salmon, Coho Salmon and Rainbow Trout in Chile: Promoting a Preventive Approach in Aquamedicine. Biology, 1066.
18. Munro L. A. & Wallace, I. S., 2017. Scottish fish farm production seruvery 2017. Scottish Government, 2018.
19. Ogwang, Tomson. A convenient method of decomposing the Gini index by population subgroups. Journal of Official Statistics 30.1 (2014): 91.
20. Quinn, B., McEneff, G., Schmidt, W. 2015. Pharmaceuticals in the Irish Aquatic Environment: The Assessment and Potential Human Impact of Exposure to Pharmaceuticals on Marine and Freshwater Bivalves. EPA (Ireland) report 143. 40pp.
21. Rehulka, J. 2003.Haematological analyses in rainbow trout Oncorhynchus mykiss affected by viral haemorrhagic septicaemia (VHS). Dis. Aquat. Organ. 56, 185-193.
22. Sandnes K. L. & Waagbo, R. 1988. Normal ranges of some blood chemistry parameters in adult farmed Atlantic salmon, Salmo salar. Journal of Fish Biology (1988) 32(1) 129-136.
23. Scikit-learn. (n.d.). scikit learn. Retrieved from scikit-learn.org: https://scikit-learn.org/stable/modules/generated/sklearn.model_selection.GridSearchCV.html
24. Steinbach, C. et al. 2014. The sub-lethal effects and tissue concentration of the human pharmaceutical atenolol in rainbow trout (Oncorhynchus mykiss). Sci. Total Environ. 497-498, 209-218.
25. Szymanski, P. et al. 2017. A scikit-based Python environment for performing multi-label classification. ArXiv e-prints.
26. Ivan Tomek. Two modifications of cnn. IEEE Trans. Systems, Man and Cybernetics, 6:769-772, 1976.
27. SMOTE—Version 0.11.0. (n.d.). https://imbalanced-learn.org/stable/references/generated/imblearn.over_sampling.SMOTE.html
28. TomekLinks—Version 0.11.0. (n.d.). https://imbalanced-learn.org/stable/references/generated/imblearn.under_sampling.TomekLinks.html
29. sklearn.model_selection.GridSearchCV. (n.d.). Scikit-learn. https://scikit-learn.org/stable/modules/generated/sklearn.model_selection.GridSearchCV.html
30. sklearn.ensemble.StackingClassifier. (n.d.). Scikit-learn. https://scikit-learn.org/stable/modules/generated/sklearn.ensemble.StackingClassifier.html
31. scikit-multilearn. (n.d.). http://scikit.ml/api/skmultilearn.problem_transform.lp.html
32. Niteshsukhwani. (n.d.). GitHub—niteshsukhwani/MLSMOTE: Multi label Synthetic Minority Over-sampling Technique (MLSMOTE). GitHub. https://github.com/niteshsukhwani/MLSMOTE
33. forestci API—forestci 0.6 documentation. (n.d.). https://contrib.scikit-learn.org/forest-confidence-interval/api.html

1. A method for determining the health status of at least one fish in a population of fish, comprising the steps of:
(a) analysing one or more first samples collected from at least one fish of the population of fish to determine the amount of at least one analyte selected from: chloride; iron; sodium; aspartate aminotransferase; total cholesterol;
potassium; glucose; creatine kinase; lactate dehydrogenase; alanine aminotransferase; lactate; zinc; phosphate; albumin; creatine kinase-myocardial band; lipase; calcium; and magnesium present in the sample to determine a test profile; and
(b) comparing the amount of the at least one analyte present in the test profile with a reference profile;
wherein a difference in the amount of the at least one analyte in the test profile as compared to the reference profile indicates the health status of the at least one fish in a population of fish.

2. A method of monitoring the health status of a population of fish over time, wherein the method comprises:
(a) analysing a first sample collected from at least one fish of the population of fish to determine the amount of at least one analyte selected from the group consisting of: chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium present in the sample to determine a first test profile; and
(b) analysing at least a second sample collected from at least one fish of the population of fish to determine the amount of the same at least one analyte present in the at least second sample to determine a second test profile;
(c) comparing the second test profile with the first test profile;
wherein a difference second test profile as compared to the first test profile indicates the health status of the population of fish has changed.

3. A method of diagnosing a fish or a population of fish with a condition or a disease, the method comprising:
(a) analysing a first sample collected from at least one fish of a population of fish to determine the amount of at least one analyte selected from: chloride; iron;
sodium; aspartate aminotransferase; total cholesterol; potassium; glucose;
creatine kinase; lactate dehydrogenase; alanine aminotransferase; lactate; zinc;
phosphate; albumin; creatine kinase-myocardial band; lipase; calcium; and
magnesium present in the sample to determine a test profile;
(b) comparing the amount of the at least one analyte present in the test profile with a reference profile;
wherein a difference in the amount of the at least one analyte in the test profile as compared to the reference profile indicates the at least one fish has a condition or a disease.

4. A method for monitoring progression of a condition or disease in a population of fish, comprising the steps of:
(a) analysing one or more first samples collected from at least one fish of the population of fish to determine the amount of at least one analyte, the analyte preferably being selected from: chloride; iron; sodium; aspartate aminotransferase;
total cholesterol; potassium; glucose; creatine kinase; lactate dehydrogenase;
alanine aminotransferase; lactate; zinc; phosphate; albumin; creatine kinase-myocardial band; lipase; calcium; and magnesium to determine a first test profile; and
(b) analysing at least one later sample collected from at least one fish of the population of fish at a at least one later time point to determine the amount of the same at least one analyte present in the least one later sample to determine at least one later test profile;
determining from a comparison between the first test profile and the second test profile the progression of the condition or the disease.

5. The method according to any one of claims 1-4, wherein the method comprises analysing a sample collected from the at least one fish of the population of fish to determine the amount of aspartate aminotransferase and optionally one or more of chloride; iron; sodium; total cholesterol; potassium; glucose; creatine kinase; lactate dehydrogenase; alanine aminotransferase; lactate; zinc; phosphate; albumin; creatine kinase-myocardial band; lipase; calcium; and magnesium.

6. The method according to any one of claims 1-4, wherein the method comprises analysing a sample collected from the at least one fish of the population of fish to determine the amount of iron and optionally one or more of chloride; aspartate aminotransferase; sodium; total cholesterol; potassium; glucose; creatine kinase; lactate dehydrogenase; alanine aminotransferase; lactate; zinc; phosphate; albumin; creatine kinase-myocardial band; lipase; calcium; and magnesium.

7. The method according to any one of claims 1-4, wherein the method comprises analysing a sample collected from the at least one fish of the population of fish to determine the amount of glucose and optionally one or more of chloride; aspartate aminotransferase; sodium; total cholesterol; potassium; iron; creatine kinase; lactate dehydrogenase; alanine aminotransferase; lactate; zinc; phosphate; albumin; creatine kinase-myocardial band; lipase; calcium; and magnesium.

8. The method according to any one of claims 1-4, wherein the method comprises analysing a sample collected from the at least one fish of the population of fish to determine the amount of creatine kinase and optionally one or more of chloride; aspartate aminotransferase; sodium; total cholesterol; potassium; iron; glucose; lactate dehydrogenase; alanine aminotransferase; lactate; zinc; phosphate; albumin; creatine kinase-myocardial band; lipase; calcium; and magnesium.

9. The method according to any one of claims 1-4, wherein the method comprises analysing a sample collected from the at least one fish of the population of fish to determine the amount of chloride and optionally one or more of creatine kinase; aspartate aminotransferase; sodium; total cholesterol; potassium; iron; glucose; lactate dehydrogenase; alanine aminotransferase; lactate; zinc; phosphate; albumin; creatine kinase-myocardial band; lipase; calcium; and magnesium.

10. The method according to any one of claims 1-4, wherein the method comprises analysing a sample collected from the at least one fish of the population of fish to determine the amount of sodium and optionally one or more of creatine kinase; aspartate aminotransferase; chloride; total cholesterol; potassium; iron; glucose; lactate dehydrogenase; alanine aminotransferase; lactate; zinc; phosphate; albumin; creatine kinase-myocardial band; lipase; calcium; and magnesium.

11. The method according to any one of claims 1-4, wherein the method comprises analysing a sample collected from the at least one fish of the population of fish to determine the amount of total cholesterol and optionally one or more of creatine kinase; aspartate aminotransferase; chloride; sodium; potassium; iron; glucose; lactate dehydrogenase; alanine aminotransferase; lactate; zinc; phosphate; albumin; creatine kinase-myocardial band; lipase; calcium; and magnesium.

12. The method according to any one of claims 1-4, wherein the method comprises analysing a sample collected from the at least one fish of the population of fish to determine the amount of any two or more of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

13. The method according to any one of claims 1-4, wherein the method comprises analysing a sample collected from the at least one fish of the population of fish to determine the amount of aspartate aminotransferase; iron; glucose; creatine kinase; chloride; zinc and total cholesterol.

14. The method according to any one of claims 1-4, wherein the method comprises analysing a sample collected from the at least one fish of the population of fish to determine the amount of aspartate aminotransferase; iron; glucose; creatine kinase; chloride; sodium and total cholesterol.

15. The method according to any one of claims 1-4, wherein the method comprises analysing a sample collected from the at least one fish of the population of fish to determine the amount of chloride; iron; sodium; aspartate aminotransferase; total cholesterol; potassium; glucose; creatine kinase; lactate dehydrogenase; alanine aminotransferase; lactate; zinc; phosphate; albumin; creatine kinase-myocardial band; lipase; calcium; and magnesium.

16. The method according to any one of claims 3-15, wherein the condition or disease is any one or more of pancreas disease, cardiomyopathy syndrome, gill issues, heart and skeletal muscle inflammation and bacterial infection.

17. The method according to claim 3 or 4, wherein an increase in the amount of creatinine kinase, creatine kinase-myocardial band, lactate dehydrogenase, alanine aminotransferase and lipase and a decrease in the amount of total cholesterol, glucose, zinc, phosphate and iron when compared to the reference profile indicates the population of fish have pancreas disease.

18. The method according to claim 3 or 4, wherein an increase in the amount of magnesium, phosphate, lactate, sodium, potassium and chloride and a decrease in the amount of glucose when compared to the reference profile indicates the population of fish have gill issues.

19. The method according to claim 3 or 4, wherein an increase in the amount of increase in the amount of alanine aminotransferase, aspartate aminotransferase, creatine kinase, creatine kinase-myocardial band, lactate, lactate dehydrogenase, magnesium, phosphate, sodium, potassium and chloride, and a decrease in the amount of calcium, albumin, total cholesterol, glucose, iron and zinc when compared to the reference profile indicates that the fish or the population of fish has gill issues associated with an environmental challenge.

20. The method according to claim 3 or 4, wherein an increase in the amount of creatine kinase-myocardial band, lactate dehydrogenase, creatine kinase, aspartate aminotransferase, and a decrease in albumin, zinc and iron when compared to the reference profile indicates the population of fish have heart and skeletal muscle inflammation.

21. The method according to claim 3 or 4, wherein an increase in the amount of lactate, sodium, potassium and chloride and a decrease in the amount of albumin and zinc when compared to the reference profile indicates the population of fish have cardiomyopathy syndrome.

22. The method according to claim 3 or 4, wherein an increase in the amount of lactate dehydrogenase and lactate and a decrease in the amount of albumin, total cholesterol and iron when compared to the reference profile indicates the population of fish have a bacterial infection.

23. A method for determining a welfare score for a population of fish, wherein the method comprises:
(a) determining the health status of a plurality of fish from a population of fish according to any one of claims 1, 2 and 5-15;
(b) classifying each sample obtained from a plurality of fish from the population of fish into one of a plurality of health level categories; and
(c) providing a score corresponding to the proportion of fish in each health level category wherein the score is indicative of the overall population welfare.

24. The method according to claim 23, wherein the plurality of health level categories are good health, weak health or critical health.

25. A computer implemented method of training a model to stratify a population of fish according to health status, wherein the method comprises:
(a) determining an amount of at least one analyte in a plurality of samples obtained from a population of fish to determine the health status of a population of fish;
(b) producing a training dataset of analytes associated with known healthy samples and unhealthy samples based on (a); and
(c) training the model using the training dataset from (b).

26. A computer implemented method of training a model to predict a health challenge in a population of fish, wherein the method comprises:
(a) diagnosing at least one fish from a population of fish with one or more health challenges;
(b) determining an amount of at least one analyte obtained from the at least one fish from the population of fish;
(c) produce a training dataset based on (a) and (b);
(d) training the model based on training dataset from (c).

27. A computer implemented method for determining the health status of a population of fish, wherein the method comprises:
(a) measuring or providing the amount of a plurality of analytes in at least one sample obtained from a population of fish;
(b) inputting the amount of the plurality of analytes from step (a) to an algorithm which correlates analyte amount with fish health status; and
(c) assigning a health status to the at least one fish from the population of fish.

28. The computer implemented method according to claim 27, wherein the assigned health status is healthy or unhealthy.

29. The computer implemented method according to claim 27 wherein, the assigned health status is a probability value.

30. The computer implemented method according to claim 29, wherein a probability value of 0 to 0.3 is a fish with good health, a probability value of 0.3 to 0.7 is a fish with weak health and a probability value of 0.7 to 1 is a fish with critical health.

31. A computer implemented method for predicting a health challenge in at least one fish of a population of fish; wherein the method comprises:
a) measuring the amount of a plurality of analytes from at least one sample obtained from at least one fish of a population of fish;
b) inputting the amount of the plurality of analytes from step (a) to an algorithm which correlates analyte amount with fish health challenges; and
c) assigning a health challenge to the at least one fish of a population of fish.

32. A computer implemented method according to claim 31, wherein the health challenge is pancreas disease, gill issues, cardiomyopathy, heart and skeletal muscle inflammation and/or bacterial infection.

33. The computer implemented method according to any one of claims 27-32, wherein the plurality of analytes is any one or more of chloride, iron, sodium, aspartate aminotransferase, total cholesterol, potassium, glucose, creatine kinase, lactate dehydrogenase, alanine aminotransferase, lactate, zinc, phosphate, albumin, creatine kinase-myocardial band, lipase, calcium, and magnesium.

34. The computer implemented method according to any one of claims 27-33, wherein the algorithm is a trained algorithm, optionally wherein the trained algorithm is trained according to any one of claims 25 or 26.

35. A computer implemented method for determining a welfare score, wherein the method comprises:
(a) determining the health status of at least one fish from a population of fish according to any one of claims 27-29;
(b) inputting the health status from (a) into an algorithm to determine a welfare score.

36. The method according to claim 35, wherein the welfare score is good health, weak health or critical health.

37. A computer implemented method for determining a fish welfare score ratio, wherein the method comprises:
(a) determining a plurality of welfare scores of according to any one of claims 35-36;
(b) inputting the plurality of welfare scores from step (a) into an algorithm to determine a population welfare value for a population of fish.

38. The method according to any preceding claim wherein the sample is blood, plasma or serum.

39. The method according to any preceding claim wherein the fish or population of fish are wild, captive or farmed fish.

40. The method according to claim 39, wherein the fish or population of fish are salmonid, sea bass, sea bream, sturgeon and/or carp.

41. A trained model that has been obtained by the method of any one of claims 25 or 26.

42. A computer program comprising instructions which, when the program is executed by a computer, cause the computer to carry out the method of determining the health status of at least one fish from a population of fish according to any one of claims 27-30, 33-34 and 38-40.

43. A computer program comprising instructions which, when the program is executed by a computer, cause the computer to carry out the method of predicting a health challenge in at least one fish from a population of fish according to any one of claims 32-34 and 38-40.

44. A computer program comprising instructions which, when the program is executed by a computer, cause the computer to carry out the method of determining a fish welfare value according to any one of claims 37-40.

45. A kit for use in a method according to any one of claims 1-40, wherein the kit comprises one or more reagents for determining the amount of the at least one analyte in a sample and instructions for use.
